SEQUENCE LISTING (12 sequences)

SEQ ID NO: 1 - protein, 246 aa, synthetic construct
  source 1..246: note = anti-GITR antibody VH
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSQVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVS

SEQ ID NO: 2 - protein, 214 aa, synthetic construct
  source 1..214: note = anti-GITR antibody VL
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIKAIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 3 - protein, 246 aa, synthetic construct
  source 1..246: note = anti-GITR antibody VH
QVQLVESGGGVVQPGRSLRLSCAASGFTFSSYGFHWVRQAPGKGLEWVAVIWYAGSNKFYADSVKGRFTISRDNSKNTLYLQMNSLRAEDTAVYYCARGGQLDYYYYYVMDVWGQGTTVTVSSQVQLVESGGGVVQPGRSLRLSCAASGFTFSSYGFHWVRQAPGKGLEWVAVIWYAGSNKFYADSVKGRFTISRDNSKNTLYLQMNSLRAEDTAVYYCARGGQLDYYYYYVMDVWGQGTTVTVSS

SEQ ID NO: 4 - protein, 214 aa, synthetic construct
  source 1..214: note = anti-GITR antibody VL
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKDIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 5 - protein, 244 aa, synthetic construct
  source 1..244: note = anti-GITR antibody VH
VQLVESGGGVVQPGRSLRLSCAASGFTFSSYGMHWVRQAPGKGLEWVAVIWYAGSNKYYADSVKGRFTISRDNSKNTLYLQMNSLRAEDTAVYYCARGGRIAVAFYYSMDVWGQGTTVTVSSVQLVESGGGVVQPGRSLRLSCAASGFTFSSYGMHWVRQAPGKGLEWVAVIWYAGSNKYYADSVKGRFTISRDNSKNTLYLQMNSLRAEDTAVYYCARGGRIAVAFYYSMDVWGQGTTVTVSS

SEQ ID NO: 6 - protein, 214 aa, synthetic construct; identical to SEQ ID NO: 4
  source 1..214: note = anti-GITR antibody VL

SEQ ID NO: 7 - protein, 898 aa, synthetic construct
  source 1..898: note = anti-GITR antibody heavy chain
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGQVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 8 - protein, 428 aa, synthetic construct
  source 1..428: note = anti-GITR antibody light chain
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 906 aa, synthetic construct
  source 1..906: note = anti-GITR antibody heavy chain
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGQVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 428 aa, synthetic construct; identical to SEQ ID NO: 8
  source 1..428: note = anti-GITR antibody light chain

SEQ ID NO: 11 - protein, 113 aa, synthetic construct [sequence omitted]

SEQ ID NO: 12 - protein, 107 aa, synthetic construct [sequence omitted]

(19) United States
(12) Patent Application Publication
(10) Pub. No.: US 2025/0059610 A1
(43) Pub. Date: Feb. 20, 2025

(54) METHODS OF TREATING TUMOR

(71) Applicant: Bristol-Myers Squibb Company, Princeton, NJ (US)

(72) Inventors: Prabhu Seshaiyer BHAGAVATHEESWARAN, Hamden, CT (US); Nicholas Allan John BOTWOOD, Princeton, NJ (US); Han CHANG, West Windsor, NJ (US); Yali FU, Princeton, NJ (US); William J. GEESE, Pipersville, PA (US); George A. GREEN, Newton, NJ (US); Diane HEALEY, Madison, CT (US); Sabine MAIER, Lawrenceville, NJ (US); Faith E. NATHAN, Moorestown, NJ (US); Abderrahim OUKESSOU, Skillman, NJ (US); Giovanni SELVAGGI, Brooklyn, NY (US); Joseph Daniel SZUSTAKOWSKI, Pennington, NJ (US)

(73) Assignee: Bristol-Myers Squibb Company, Princeton, NJ (US)

(21) Appl. No.: 18/818,366

(22) Filed: Aug. 28, 2024

Related U.S. Application Data
(63) Continuation of application No. 18/063,015, filed on Dec. 7, 2022.
Application No. 18/063,015 is a continuation of application No. 16/499,540, filed on Sep. 30, 2019, now abandoned.
(60) Provisional application No. 62/582,146, filed on Nov. 6, 2017.
(60) Provisional application No. 62/479,817, filed on Mar. 31, 2017.

(51) Int. Cl. C12Q 1/6886

(52) U.S. Cl. CPC C12Q 1/6886; C12Q 2600/106; C12Q 2600/156

(57) ABSTRACT

The disclosure provides a method for treating a subject afflicted with a tumor, e.g., lung cancer, having a high tumor mutation burden (TMB) status comprising administering to the subject an immunotherapy, e.g., an anti-PD-I antibody or antigen-binding portion thereof. The present disclosure also provides a method for identifying a subject suitable for an immunotherapy, e.g., a treatment with an anti-PD-I antibody or antigen-binding portion thereof, comprising measuring a TMB status of a biological sample of the subject. A high TMB status identifies the patient as suitable for treatment with an anti-PD-I antibody or antigen-binding portion thereof. The TMB status can be determined by sequencing nucleic acids in the tumor and identifying a genomic alteration, e.g., a somatic nonsynonymous mutation, in the sequenced nucleic acids.

### FIELD OF THE DISCLOSURE

The present disclosure provides a method for treating a subject afflicted with a tumor having a high tumor mutational burden (TMB) status comprising administering to the subject an immunotherapy. In some embodiments, the immunotherapy comprises an antibody or an antigen-binding fragment thereof. In certain embodiments, the immunotherapy comprises an anti-PD-1 antibody or antigen-binding portion thereof or an anti-PD-L1 antibody or antigen-binding portion thereof.

### REFERENCE TO SEQUENCE LISTING SUBMITTED ELECTRONICALLY

The content of the electronically submitted sequence listing in ASCII text file (Name: 3338066PC02_sequence_ST25.txt; Size: 38,235 bytes; and Date of Creation: Mar. 30, 2018) is incorporated herein by reference in its entirety.

### BACKGROUND OF THE DISCLOSURE

Human cancers harbor numerous genetic and epigenetic alterations, generating neoantigens potentially recognizable by the immune system (Sjoblom et al., Science (2006) 314 (5797): 268-274). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

Until recently, cancer immunotherapy had focused substantial effort on approaches that enhance anti-tumor immune responses by adoptive-transfer of activated effector cells, immunization against relevant antigens, or providing non-specific immune-stimulatory agents such as cytokines. In the past decade, however, intensive efforts to develop specific immune checkpoint pathway inhibitors have begun to provide new immunotherapeutic approaches for treating cancer, including the development of antibodies such as nivolumab and pembrolizumab (formerly lambrolizumab; USAN Council Statement, 2013) that bind specifically to the Programmed Death-1 (PD-1) receptor and block the inhibitory PD-1/PD-1 ligand pathway (Topalian et al., 2012a, b; Topalian et al., 2014; Hamid et al., 2013; Hamid and Carvajal, 2013; McDermott and Atkins, 2013).

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to downregulate T cell activation and cytokine secretion upon binding to PD-1. Inhibition of the PD-1/PD-L1 interaction mediates potent antitumor activity in preclinical models (U.S. Pat. Nos. 8,008,449 and 7,943,743), and the use of antibody inhibitors of the PD-1/PD-L1 interaction for treating cancer has entered clinical trials (Brahmer et al., 2010; Topalian et al., 2012a; Topalian et al., 2014; Hamid et al., 2013; Brahmer et al., 2012; Flies et al., 2011; Pardoll, 2012; Hamid and Carvajal, 2013).

Nivolumab (formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014). Nivolumab has shown activity in a variety of advanced solid tumors, including renal cell carcinoma (renal adenocarcinoma, or hypernephroma), melanoma, and non-small cell lung cancer (NSCLC) (Topalian et al., 2012a; Topalian et al., 2014; Drake et al., 2013; WO 2013/173223).

The immune system and response to immuno-therapy are complex. Additionally, anti-cancer agents can vary in their effectiveness based on the unique patient characteristics. Accordingly, there is a need for targeted therapeutic strategies that identify patients who are more likely to respond to a particular anti-cancer agent and, thus, improve the clinical outcome for patients diagnosed with cancer.

### SUMMARY OF THE DISCLOSURE

The present disclosure provides a method for treating a subject afflicted with a tumor comprising administering to the subject a therapeutically effective amount of an anti-PD-1 antibody or antigen-binding portion thereof, wherein the tumor has a tumor mutational burden (TMB) status that is a high TMB. In some embodiments, the method further comprises measuring the TMB status of a biological sample obtained from the subject.

The present disclosure also provides a method of identifying a subject suitable for a therapy of an anti-PD-1 antibody or antigen-binding portion thereof comprising measuring a TMB status of a biological sample of the subject, wherein the TMB status is a high TMB thereby the subject is identified as being suitable for the therapy of an anti-PD-1 antibody or antigen-binding portion thereof. In one embodiment, the method further comprises administering to the subject the anti-PD-1 antibody or antigen-binding portion thereof.

In some embodiments, the TMB status is determined by sequencing nucleic acids in the tumor and identifying a genomic alteration in the sequenced nucleic acids. In some embodiments, the genomic alteration comprises one or more somatic mutations. In some embodiments, the genomic alteration comprises one or more nonsynonymous mutations. In a particular embodiment, the genomic alteration comprises one or more missense mutations. In other particular embodiments, the genomic alteration comprises one or more alterations selected from the group consisting of a base pair substitution, a base pair insertion, a base pair deletion, a copy number alteration (CNA), a gene rearrangement, and any combination thereof.

In particular embodiments, the TMB status is determined by genome sequencing, exome sequencing, and/or genomic profiling. In one embodiment, the genomic profile comprises at least 300 genes, at least 305 genes, at least 310 genes, at least 315 genes, at least 320 genes, at least 325 genes, at least 330 genes, at least 335 genes, at least 340 genes, at least 345 genes, at least 350 genes, at least 355 genes, at least 360 genes, at least 365 genes, at least 370 genes, at least 375 genes, at least 380 genes, at least 385 genes, at least 390 genes, at least 395 genes, or at least 400 genes. In a particular embodiment, the genomic profile comprises at least 325 genes.

In one embodiment, the genomic profile comprises one or more genes selected from the group consisting of ABL1, BRAF, CHEK1, FANCC, GATA3, JAK2, MITF, PDCD1LG2, RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCE, GID4 (C17orf39), KAT6A (MYST3), MRE11A, PDK1, RNF43, SYK, AKT2, BRIP1, CRKL, FANCG, GLI1, KDM5A, MSH2, PIK3C2B, ROS1, TAF1, AKT3, BTG1, CRLF2, FANCL, GNA11, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf30 (EMSY), (TCF, FAT1, GNAQ, KDR, MUTYH, PIK3CG, RUNX1T1, TERT (promoter only), APC, CARD11, CTNNA1, FBXW7, GNAS, KEAP1, MYC, PIK3R1, SDHA, TET2, AR, CBFB, CTNNB1, FGF10, GPR124, KEL, MYCL (MYCL1), PIK3R2, SDHB, TGFBR2, ARAF, CBL, CUL3, FGF14, GRIN2A, KIT, MYCN, PLCG2, SDHC, TNFAIP3, ARFRP1, COND1, CYLD, FGF19, GRM3, KLHL6, MYD88, PMS2, SDHD, TNFRSF14, ARID1A, COND2, DAXX, FGF23, GSK3B, KMT2A (MLL), NF1, POLD1, SETD2, TOP1, ARID1B, CCND3, DDR2, FGF3, H3F3A, KMT2 (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274, DNMT3A, FGF6, HNF1A, KRAS, NFKB1A, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP90AA1, LYN, NOTCH2, PRKC1, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLON, IGF1R, MAP2K1, NRAS, PTCH1, SNCA1P, WISP3, AXL, CDK6, EPHB1, FLT1, IGF2, MAP2K2, NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, QK1, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFI1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, STAT3, and any combination thereof.

In some embodiments, the methods further comprise identifying a genomic alteration in one or more of ETV4, TMPRSS2, ETV5, BCR, ETV1, ETV6, and MYB.

In some embodiments, the high TMB has a score of at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, or at least 500. In other embodiments, the high TMB has a score of at least 215, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, or at least 250. In a particular embodiment, the high TMB has a score of at least 243.

In some embodiments, the methods further comprise comparing the subject's TMB status to a reference TMB value. In one embodiment, the subject's TMB status is within the highest fractile of the reference TMB value. In another embodiment, the subject's TMB status is within the top tertile of the reference TMB value.

In some embodiments, the biological sample is a tumor tissue biopsy, e.g., a formalin-fixed, paraffin-embedded tumor tissue or a fresh-frozen tumor tissue. In other embodiments, the biological sample is a liquid biopsy. In some embodiments, the biological sample comprises one or more of blood, serum, plasma, exoRNA, circulating tumor cells, ctDNA, and cfDNA.

In some embodiments, the subject has a tumor with a high neoantigen load. In other embodiments, the subject has an increased T-cell repertoire.

In some embodiments, the tumor is lung cancer. In one embodiment, the lung cancer is non-small cell lung cancer (NSCLC). The NSCLC can have a squamous histology or a non-squamous histology.

In other embodiments, the tumor is selected from renal cell carcinoma, ovarian cancer, colorectal cancer, gastrointestinal cancer, esophageal cancer, bladder cancer, lung cancer, and melanoma.

In some embodiments, the anti-PD-1 antibody or antigen-binding portion thereof cross-competes with nivolumab for binding to human PD-1. In other embodiments, the anti-PD-1 antibody or antigen-binding portion thereof binds to the same epitope as nivolumab. In some embodiments, the anti-PD-1 antibody is a chimeric antibody, a humanized antibody, a human monoclonal antibody, or an antigen-binding portion thereof. In other embodiments, the anti-PD-1 antibody or antigen-binding portion thereof comprises a heavy chain constant region of a human IgG1 isotype or a human IgG4 isotype. In particular embodiments, the anti-PD-1 antibody or antigen-binding portion thereof is nivolumab or pembrolizumab.

In some embodiments, the anti-PD-1 antibody or antigen-binding portion thereof is administered at a dose ranging from 0.1 mg/kg to 10.0 mg/kg body weight once every 2, 3, or 4 weeks. In one embodiment, the anti-PD-1 antibody or antigen-binding portion thereof is administered at a dose of 5 mg/kg or 10 mg/kg body weight once every 3 weeks. In another embodiment, the anti-PD-1 antibody or antigen-binding portion thereof is administered at a dose of 5 mg/kg body weight once every 3 weeks. In yet another embodiment, the anti-PD-1 antibody or antigen-binding portion thereof is administered at a dose of 3 mg/kg body weight once every 2 weeks.

In some embodiments, the anti-PD-1 antibody or antigen-binding portion thereof is administered as a flat dose. In one embodiment, the anti-PD-1 antibody or antigen-binding portion thereof is administered as a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, or at least about 550 mg. In another embodiment, the anti-PD-1 antibody or antigen-binding portion thereof is administered as a flat dose about once every 1, 2, 3, or 4 weeks.

In some embodiments, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the administration.

In other embodiments, the subject exhibits an overall survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the administration.

In yet other embodiments, the subject exhibits an objective response rate of at least about 30%, about 35%, about 40%, about 45%, about 50%, about 55%, about 60%, about 65%, about 70%, about 75%, about 80%, about 85%, about 90%, about 95%, or about 100%.

In some embodiments, the tumor has at least about 1%, about 5%, about 10%, about 15%, about 20%, about 25%, about 30%, about 35%, about 40%, about 45%, or about 50% PD-L1 expression.

Other features and advantages of the instant disclosure will be apparent from the following detailed description and examples which should not be construed as limiting. The contents of all cited references, including scientific articles, newspaper reports, GenBank entries, patents and patent applications cited throughout this application are expressly incorporated herein by reference.

### EMBODIMENTS

Embodiment 1. A method for treating a subject afflicted with a tumor comprising administering to the subject a therapeutically effective amount of an antibody or antigen-binding portion thereof that binds specifically to a Programmed Death-1 (PD-1) receptor and inhibits PD-1 activity (“an anti-PD-1 antibody or antigen-binding portion thereof”), wherein the tumor has a tumor mutational burden (TMB) status that is a high TMB.

Embodiment 2. The method of Embodiment 1, further comprising measuring the TMB status of a biological sample obtained from the subject.

Embodiment 3. A method of identifying a subject suitable for a therapy of an anti-PD-1 antibody or antigen-binding portion thereof comprising measuring a TMB status of a biological sample of the subject, wherein the TMB status is a high TMB and wherein the subject is identified as being suitable for the therapy of an anti-PD-1 antibody or antigen-binding portion thereof.

Embodiment 4. The method of Embodiment 3, further comprising administering to the subject the anti-PD-1 antibody or antigen-binding portion thereof.

Embodiment 5. The method of any one of Embodiments 1 to 4, wherein the TMB status is determined by sequencing nucleic acids in the tumor and identifying a genomic alteration in the sequenced nucleic acids.

Embodiment 6. The method of Embodiment 5, wherein the genomic alteration comprises one or more somatic mutations.

Embodiment 7. The method of Embodiment 5 or 6, wherein the genomic alteration comprises one or more nonsynonymous mutations.

Embodiment 8. The method of any one of Embodiments 5 to 7, wherein the genomic alteration comprises one or more missense mutations.

Embodiment 9. The method of any one of Embodiments 5 to 8, wherein the genomic alteration comprises one or more alterations selected from the group consisting of a base pair substitution, a base pair insertion, a base pair deletion, a copy number alteration (CNAs), a gene rearrangement, and any combination thereof.

Embodiment 10. The method of any one of Embodiments 1 to 9, wherein the high TMB has a score of at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, or at least 500.

Embodiment 11. The method of any one of Embodiments 1 to 9, wherein the high TMB has a score of at least 215, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, or at least 250.

Embodiment 12. The method of any one of Embodiments 1 to 11, wherein the high TMB has a score of at least 243.

Embodiment 13. The method of any one of Embodiments 1 to 12, further comprising comparing the subject's TMB status to a reference TMB value.

Embodiment 14. The method of Embodiment 13, wherein the subject's TMB status is within the highest fractile of the reference TMB value.

Embodiment 15. The method of Embodiment 13, wherein the subject's TMB status is within the top tertile of the reference TMB value.

Embodiment 16. The method of any one of Embodiments 1 to 15, wherein the biological sample is a tumor tissue biopsy.

Embodiment 17. The method of Embodiment 16, wherein the tumor tissue is a formalin-fixed, paraffin-embedded tumor tissue or a fresh-frozen tumor tissue.

Embodiment 18. The method of any one of Embodiments 1 to 15, wherein the biological sample is a liquid biopsy.

Embodiment 19. The method of any one of Embodiments 1 to 15, wherein the biological sample comprises one or more of blood, serum, plasma, exoRNA, circulating tumor cells, ctDNA, and cfDNA.

Embodiment 20. The method of any one of Embodiments 1 to 19, wherein the TMB status is determined by genome sequencing.

Embodiment 21. The method of any one of Embodiments 1 to 19, wherein the TMB status is determined by exome sequencing.

Embodiment 22. The method of any one of Embodiments 1 to 19, wherein the TMB status is determined by genomic profiling.

Embodiment 23. The method of Embodiment 22, wherein the genomic profile comprises at least 300 genes, at least 305 genes, at least 310 genes, at least 315 genes, at least 320 genes, at least 325 genes, at least 330 genes, at least 335 genes, at least 340 genes, at least 345 genes, at least 350 genes, at least 355 genes, at least 360 genes, at least 365 genes, at least 370 genes, at least 375 genes, at least 380 genes, at least 385 genes, at least 390 genes, at least 395 genes, or at least 400 genes.

Embodiment 24. The method of Embodiment 22, wherein the genomic profile comprises at least 325 genes.

Embodiment 25. The method of any one of Embodiments 22 to 24, wherein the genomic profile comprises one or more genes selected from the group consisting of ABL1, BRAF, CHEK1, FANCC, GATA3, JAK2, MITF, PDCD1LG2, RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCE, GID4 (C17orf39), KAT6A (MYST3), MRE11A, PDK1, RNF43, SYK, AKT2, BRIP1, CRKL, FANCG, GLI1, KDM5A, MSH2, PIK3C2B, ROS1, TAF1, AKT3, BTG1, CRLF2, FANCL, GNA1I, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf30 (EMSY), CTCF, FAT1, GNAQ, KDR, MUTYH, PIK3CG, RUNX1T1, TERT (promoter only), APC, CARD11, CTNNA1, FBXW7, GNAS, KEAP1, MYC, PIK3R1, SDHA, TET2, AR, CBFB, CTNNB1, FGF10, GPR124, KEL, MYCL (MYCL1), PIK3R2, SDHB, TGFBR2, ARAF, CBL, CUL3, FGF14, GRIN2A, KIT, MYCN, PLCG2, SDHC, TNFAIP3, ARFRP1, COND1, CYLD, FGF19, GRM3, KLHL6, MYD88, PMS2, SDHD, TNFRSF14, ARID1A, CCND2, DAXX, FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274, DNMT3A, FGF6, HNF1A, KRAS, NFKB1A, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP90AA1, LYN, NOTCH2, PRKC1, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1, NRAS, PTCH1, SNCA1P, WISP3, AXL, CDK6, EPHB1, FLT1, IGF2, MAP2K2, NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, QK1, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFI1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, STAT3, and any combination thereof.

Embodiment 26. The method of any one of Embodiments 1 to 25, further comprising identifying a genomic alteration in one or more of ETV4, TMPRSS2, ETV5, BCR, ETV1, ETV6, and MYB.

Embodiment 27. The method of any one of Embodiments 1 to 26, wherein the subject has a tumor with a high neoantigen load.

Embodiment 28. The method of any one of Embodiments 1 to 27, wherein the subject has an increased T-cell repertoire.

Embodiment 29. The method of any one of Embodiments 1 to 28, wherein the tumor is lung cancer.

Embodiment 30. The method of Embodiment 29, wherein the lung cancer is non-small cell lung cancer (NSCLC).

Embodiment 31. The method of Embodiment 30, wherein the NSCLC has a squamous histology.

Embodiment 32. The method of Embodiment 30, wherein the NSCLC has a non-squamous histology.

Embodiment 33. The method of any one of Embodiments 1 to 28, wherein the tumor is selected from renal cell carcinoma, ovarian cancer, colorectal cancer, gastrointestinal cancer, esophageal cancer, bladder cancer, lung cancer, and melanoma.

Embodiment 34. The method of any one of Embodiments 1 to 33, wherein the anti-PD-1 antibody or antigen-binding portion thereof cross-competes with nivolumab for binding to human PD-1.

Embodiment 35. The method of any one of Embodiments 1 to 34, wherein the anti-PD-1 antibody or antigen-binding portion thereof binds to the same epitope as nivolumab.

Embodiment 36. The method of any one of Embodiments 1 to 35, wherein the anti-PD-1 antibody is a chimeric antibody, a humanized antibody, a human monoclonal antibody, or an antigen-binding portion thereof.

Embodiment 37. The method of any one of Embodiments 1 to 36, wherein the anti-PD-1 antibody or antigen-binding portion thereof comprises a heavy chain constant region of a human IgG1 isotype or a human IgG4 isotype.

Embodiment 38. The method of any one of Embodiments 1 to 37, wherein the anti-PD-1 antibody or antigen-binding portion thereof is nivolumab.

Embodiment 39. The method of any one of Embodiments 1 to 37, wherein the anti-PD-1 antibody or antigen-binding portion thereof is pembrolizumab.

Embodiment 40. The method of any one of Embodiments 1 to 39, wherein the anti-PD-1 antibody or antigen-binding portion thereof is administered at a dose ranging from 0.1 mg/kg to 10.0 mg/kg body weight once every 2, 3, or 4 weeks.

Embodiment 41. The method of any one of Embodiments 1 to 40, wherein the anti-PD-1 antibody or antigen-binding portion thereof is administered at a dose of 5 mg/kg or 10 mg/kg body weight once every 3 weeks.

Embodiment 42. The method of any one of Embodiments 1 to 41, wherein the anti-PD-1 antibody or antigen-binding portion thereof is administered at a dose of 5 mg/kg body weight once every 3 weeks.

Embodiment 43. The method of any one of Embodiments 1 to 40, wherein the anti-PD-1 antibody or antigen-binding portion thereof is administered at a dose of 3 mg/kg body weight once every 2 weeks.

Embodiment 44. The method of any one of Embodiments 1 to 39, wherein the anti-PD-1 antibody or antigen-binding portion thereof is administered as a flat dose.

Embodiment 45. The method of Embodiment 44, wherein the anti-PD-1 antibody or antigen-binding portion thereof is administered as a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, or at least about 550 mg.

Embodiment 46. The method of Embodiment 44 or 45, wherein the anti-PD-1 antibody or antigen-binding portion thereof is administered as a flat dose about once every 1, 2, 3, or 4 weeks.

Embodiment 47. The method of any one of Embodiments 1 to 46, wherein the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the administration.

Embodiment 48. The method of any one of Embodiments 1 to 47, wherein the subject exhibits an overall survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the administration.

Embodiment 49. The method of any one of Embodiments 1 to 48, wherein the subject exhibits an objective response rate of at least about 30%, about 35%, about 40%, about 45%, about 50%, about 55%, about 60%, about 65%, about 70%, about 75%, about 80%, about 85%, about 90%, about 95%, or about 100%.

Embodiment 50. The method of any one of Embodiments 1 to 49, wherein the tumor has at least about 1%, about 5%, about 10%, about 15%, about 20%, about 25%, about 30%, about 35%, about 40%, about 45%, or about 50% PD-L1 expression.

Embodiment 51. A method of identifying a subject suitable for a cancer therapy comprising measuring a TMB status of a tumor sample of the subject using a platform, wherein the TMB status is determined by sequencing cancer-related genes and select introns.

Embodiment 52. The method of Embodiment 51, wherein the cancer therapy comprises administering to the subject a therapeutically effective amount of an antibody or antigen-binding portion thereof that binds specifically to a Programmed Death-1 (PD-1) receptor and inhibits PD-1 activity (“an anti-PD-1 antibody or antigen-binding portion thereof”).

Embodiment 53. The method of Embodiment 51 or 52, wherein the tumor is selected from renal cell carcinoma, ovarian cancer, colorectal cancer, gastrointestinal cancer, esophageal cancer, bladder cancer, lung cancer, and melanoma.

Embodiment 54. The method of any one of Embodiments 1 to 53, wherein the TMB status is measured using a FOUNDATIONONE® assay.

### BRIEF DESCRIPTION OF THE DRAWINGS

FIG. 1 provides a consolidated standards of reporting trials (CONSORT) diagram of patient disposition.

FIG. 2 shows the study design.

FIG. 3 shows progression-free survival (PFS) in patients with ≥5% PD-L1 expression.

FIG. 4 shows progression-free survival (PFS) in all randomized patients.

FIG. 5 shows overall survival (OS) in patients with ≥5% PD-L1 expression.

FIG. 6 shows overall survival (OS) in all randomized patients.

FIG. 7 shows progression-free survival (PFS) in all randomized patients by subgroup. ECOG PS denotes Eastern Cooperative Oncology Group performance-status.

FIG. 8 shows overall survival (OS) in all randomized patients by subgroup. ECOG PS denotes Eastern Cooperative Oncology Group performance-status.

FIG. 9 shows progression-free survival (PFS) in evaluable patients with high tumor mutation burden (TMB).

FIG. 10 shows progression-free survival (PFS) in evaluable patients with low or medium tumor mutation burden (TMB).

FIG. 11 shows overall survival (OS) in evaluable patients with high tumor mutation burden (TMB).

FIG. 12 shows overall survival (OS) in evaluable patients with low or medium tumor mutation burden (TMB).

FIG. 13 shows tumor burden analysis using total exome mutations and a gene panel.

FIG. 14 shows progression-free survival (PFS) in patients evaluable for tumor mutation burden (TMB).

FIG. 15 shows overall survival (OS) in patients evaluable for tumor mutation burden (TMB).

FIG. 16 shows progression-free survival (PFS) by tumor mutation burden (TMB) tertile in the nivolumab arm.

FIG. 17 shows progression-free survival (PFS) by tumor mutation burden (TMB) tertile in the chemotherapy arm.

FIG. 18 shows analysis of the association between tumor mutation burden (TMB) and PD-L1 expression in evaluable patients.

FIG. 19 shows overall response rate (ORR) by tumor mutation burden (TMB) and PD-L1 expression.

FIG. 20 shows partial response (PR) and complete response (CR) by tumor mutation burden (TMB) tertile in evaluable patients.

FIG. 21 shows the experimental design of tumor mutation burden (TMB) analysis of samples of 44 patients. WES: whole exome sequencing; F1: FOUNDATIONONE® sequencing.

FIG. 22 shows the high correlation between the tumor mutation burden (TMB) by FOUNDATIONONE® sequencing (F1) and by whole exome sequencing (WES). The shaded area represents the 95% confidence interval bounds, as calculated using the bootstrap (quantile) method. The horizontal dashed line shows the equivalent F1 TMB level (7.64 somatic mutations per megabase). The vertical dashed line shows the arbitrary WES TMB value set to median (148 missense mutations).

FIG. 23 is a schematic representation of a clinical trial protocol directed to the treatment of SCLC using an anti-PD-1 antibody, e.g., nivolumab, monotherapy or a combination therapy comprising an anti-PD-1 antibody, e.g., nivolumab, and an anti-CTLA-4 antibody, e.g., ipilimumab.

FIG. 24 is a schematic representation illustrating the methods and sample flow for exploratory TMB analysis.

FIGS. 25A-25D are graphical representations of progression free survival (PFS;

FIGS. 25A and 25C) and overall survival (OS; FIGS. 25B and 25D) for subjects treated with an anti-PD-1 antibody, e.g., nivolumab, monotherapy (FIGS. 25A and 25B) or a combination therapy comprising an anti-PD-1 antibody, e.g., nivolumab and an anti-CTLA-4 antibody, e.g., ipilimumab (FIGS. 25C and 25D). PFS and OS for ITT patients and TMB-evaluable patients are overlaid as indicated (FIGS. 25A-25D).

FIGS. 26A-26C are graphical representations of the TMB distribution for subjects in the SCLC clinical trial, described herein (FIG. 26A), the pooled SCLC study subjects (FIG. 26B) and the pooled subjects from a previous clinical trial directed to the treatment of non-small cell lung cancer (FIG. 26C).

FIG. 27 is a bar graph showing the overall response rate (ORR) for all TMB-evaluable subjects treated with an anti-PD-1 antibody, e.g., nivolumab or an anti-PD-1 antibody, e.g., nivolumab and an anti-CTLA-4 antibody, e.g., ipilimumab and for the same subjects stratified by TMB status (low, medium, or high).

FIGS. 28A-28B are graphical representations of the TMB distribution for subjects treated with either an anti-PD-1 antibody, e.g., nivolumab monotherapy (FIG. 28A) or a combination therapy comprising an anti-PD-1 antibody, e.g., nivolumab and an anti-CTLA-4 antibody, e.g., ipilimumab (FIG. 28B), wherein the subjects are stratified by best overall response. CR=complete response; PR=partial response; SD=stable disease; PD=progressive disease; NE=not evaluated.

FIGS. 29A-29B show the progression free survival (PFS) in subjects treated with an anti-PD-1 antibody, e.g., nivolumab, monotherapy (FIG. 29A) or a combination therapy comprising an anti-PD-1 antibody, e.g., nivolumab, and an anti-CTLA-4 antibody, e.g., ipilimumab (FIG. 29B) stratified by TMB status (low, medium, or high), as indicated. One-year PFS is marked for each sample population.

FIGS. 30A-30B show the overall survival (OS) for subjects treated with an anti-PD-1 antibody, e.g., nivolumab monotherapy (FIG. 30A) or a combination therapy comprising an anti-PD-1 antibody, e.g., nivolumab, and an anti-CTLA-4 antibody, e.g., ipilimumab (FIG. 30B) stratified by TMB status (low, medium, or high), as indicated. One-year OS is marked for each sample population.

FIG. 31 shows the study design of treating NSCLC. The subjects were divided up by the PD-L1 expression status, i.e., ≥1% PD-L1 expression v. <PD-L1 expression. The subjects in each group were then divided up into three groups (1:1:1) receiving (i) an anti-PD-1 antibody (e.g., nivolumab) at a dose of 3 mg/kg q2Q and an anti-CTLA-4 antibody, e.g., ipilimumab, at a dose of mg/kg q6W (n=396 or n=187); (ii) histology-based chemotherapy (n=397 or n=186), and (iii) an anti-PD-1 antibody, e.g., nivolumab, alone at a flat dose of 240 mg q2W (n=396 or n=177). The subjects who were receiving histology-based chemotherapy were further stratified by its status, i.e., squamous (SQ) NSCLC or non-squamous (NSQ) NSCLC. The subjects with NSQ NSCLC who received a chemotherapy received pemetrexed (500 mg/m2)+cisplatin (75 mg/m2) or carboplatin (AUC 5 or 6), Q3W for ≤4 cycles, with optional pemetrexed (500 mg/m2) maintenance following chemotherapy or nivolumab (360 mg Q3W)+pemetrexed (500 mg/m2) maintenance following nivolumab+chemotherapy. The subjects with SQ NSCLC who received a chemotherapy received gemcitabine (1000 or 1250 mg/m2)+cisplatin (75 mg/m2), or gemcitabine (1000 mg/m2)+carboplatin (AUC 5), Q3W for ≤4 cycles. The TBM co-primary analysis was conducted in the subset of patients randomized to nivolumab+ipilimumab or chemotherapy who had evaluable TMB≥10 mutations/Mb.

FIG. 32 shows a scatterplot of TMB and PD-L1 Expression in all TMB-evaluable Patients. The y axis shows the number of mutations per megabase, and the x axis shows PD-L1 expression. Symbols (dots) in the scatterplot may represent multiple data points, especially for patients with <1% PD-L1 expression.

FIG. 33A shows progression-free survival with an anti-PD-1 antibody (e.g., nivolumab) plus an anti-CTLA-4 antibody (e.g., Ipilimumab) vs. chemotherapy in all randomized patients. Cl shows confidence interval; HR shows hazard ratio. FIG. 33B shows progression-free survival with an anti-PD-1 antibody (e.g., nivolumab) plus an anti-CTLA-4 antibody (e.g., Ipilimumab) vs. chemotherapy in TMB evaluable patients.

FIG. 34A shows progression-free survival of an anti-PD-1 antibody (e.g., nivolumab) plus an anti-CTLA-4 antibody (e.g., Ipilimumab) (Nivo+Ipi) vs. chemotherapy (Chemo) in patients with TMB≥10 mutations/Mb. 1-y PFS=progression-free survival at one year; *95% CI, 0.43 to 0.77. FIG. 34B shows duration of response of an anti-PD-1 antibody (e.g., nivolumab) plus an anti-CTLA-4 antibody (e.g., Ipilimumab) (Nivo+Ipi) vs. chemotherapy (Chemo) in patients with TMB≥10 mutations/Mb. DOR: duration of response; Median, DOR, mo: median month of duration of response; 1-y DOR: duration of response at one year.

FIG. 35 shows Progression-free Survival with an anti-PD-1 antibody (e.g., nivolumab) plus an anti-CTLA-4 antibody (e.g., Ipilimumab) vs. chemotherapy in patients With TMB<10 mutations/Mb.

FIG. 36A shows subgroup analyses of progression-free survival in patients with TMB≥10 mutations/Mb by PD-L1 expression ≥1%. PFS (%): percentage of progression-free survival. FIG. 36B shows subgroup analyses of progression-free survival in patients with TMB≥10 mutations/Mb by PD-L1 expression ≤1%. FIG. 36C shows subgroup analyses of progression-free survival in patients with TMB≥10 mutations/Mb in patients with squamous cell tumor histology. FIG. 36D shows subgroup analyses of progression-free survival in patients with TMB≥10 mutations/Mb in patients with non-squamous cell tumor histology. FIG. 36E shows the characteristics of the selected subgroups.

FIG. 37 shows progression-free Survival with an anti-PD-1 antibody (e.g., nivolumab) monotherapy vs. chemotherapy in patients with TMB≥13 mutations/Mb and ≥1% tumor PD-L1 expression. 95% CI is 0.95 (0.64, 1.4).

FIG. 38 shows progression-free survival with an anti-PD-1 antibody (e.g., nivolumab) plus an anti-CTLA-4 antibody (e.g., Ipilimumab) vs. an anti-PD-1 antibody (e.g., nivolumab) monotherapy and chemotherapy in patients with TMB≥10 mutations/Mb and ≥1% tumor PD-L1 expression. 95% CI is 0.62 (0.44, 0.88) for nivolumab+ipilimumab vs. chemotherapy.

### DETAILED DESCRIPTION OF THE DISCLOSURE

The present disclosure relates to methods for treating a cancer patient with a tumor having a high TMB status comprising administering to the patient an immunotherapy. In some embodiments, the immunotherapy comprises an antibody or an antigen-binding fragment thereof. In certain embodiments, the immunotherapy comprises an anti-PD-1 antibody or antigen-binding portion thereof or an anti-PD-L1 antibody or antigen-binding portion thereof. The present disclosure also relates to a method for identifying a cancer patient suitable for treatment with immunotherapy, e.g., treatment with an anti-PD-1 antibody or antigen-binding portion thereof, comprising measuring a TMB status of a biological sample of the patient.

### Terms

In order that the present disclosure can be more readily understood, certain terms are first defined. As used in this application, except as otherwise expressly provided herein, each of the following terms shall have the meaning set forth below. Additional definitions are set forth throughout the application.

“Administering” refers to the physical introduction of a composition comprising a therapeutic agent to a subject, using any of the various methods and delivery systems known to those skilled in the art. Preferred routes of administration for the immunotherapy, e.g., the anti-PD-1 antibody or the anti-PD-L1 antibody, include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include an oral, topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. For example, an adverse event can be associated with activation of the immune system or expansion of immune system cells (e.g., T cells) in response to a treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as Vu) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 may, however, have cross-reactivity to other antigens, such as PD-1 molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic or other techniques known to those skilled in the art.

A “human antibody” (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibody” and “fully human antibody” and are used synonymously.

A “humanized antibody” refers to an antibody in which some, most or all of the amino acids outside the CDRs of a non-human antibody are replaced with corresponding amino acids derived from human immunoglobulins. In one embodiment of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-PD-1 antibody binds specifically to PD-1.

An “antigen-binding portion” of an antibody (also called an “antigen-binding fragment”) refers to one or more fragments of an antibody that retain the ability to bind specifically to the antigen bound by the whole antibody.

A “cancer” refers a broad group of various diseases characterized by the uncontrolled growth of abnormal cells in the body. Unregulated cell division and growth divide and grow results in the formation of malignant tumors that invade neighboring tissues and can also metastasize to distant parts of the body through the lymphatic system or bloodstream.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Programmed Death-1” (PD-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1” (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

A “subject” includes any human or nonhuman animal. The term “nonhuman animal” includes, but is not limited to, vertebrates such as nonhuman primates, sheep, dogs, and rodents such as mice, rats and guinea pigs. In preferred embodiments, the subject is a human. The terms, “subject” and “patient” are used interchangeably herein.

The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent (e.g., the anti-PD-1 antibody). For example, a 60 kg person and a 100 kg person would receive the same dose of an antibody (e.g., 240 mg of an anti-PD-1 antibody).

The use of the term “fixed dose” with regard to a method of the disclosure means that two or more different antibodies in a single composition (e.g., anti-PD-1 antibody and anti-CTLA-4 antibody) are present in the composition in particular (fixed) ratios with each other. In some embodiments, the fixed dose is based on the weight (e.g., mg) of the antibodies. In certain embodiments, the fixed dose is based on the concentration (e.g., mg/ml) of the antibodies. In some embodiments, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg first antibody (e.g., anti-PD-1 antibody) to mg second antibody (e.g., anti-CTLA-4 antibody). For example, the 3:1 ratio of an anti-PD-1 antibody and an anti-CTLA-4 antibody can mean that a vial can contain about 240 mg of the anti-PD-1 antibody and 80 mg of the anti-CTLA-4 antibody or about 3 mg/ml of the anti-PD-1 antibody and 1 mg/ml of the anti-CTLA-4 antibody.

The term “weight-based dose” as referred to herein means that a dose that is administered to a patient is calculated based on the weight of the patient. For example, when a patient with 60 kg body weight requires 3 mg/kg of an anti-PD-1 antibody, one can calculate and use the appropriate amount of the anti-PD-1 antibody (i.e., 180 mg) for administration.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject. In preferred embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount of an anti-cancer agent preferably inhibits cell growth or tumor growth by at least about 20%, more preferably by at least about 40%, even more preferably by at least about 60%, and still more preferably by at least about 80% relative to untreated subjects. In other preferred embodiments of the disclosure, tumor regression can be observed and continue for a period of at least about 20 days, more preferably at least about 40 days, or even more preferably at least about 60 days. Notwithstanding these ultimate measurements of therapeutic effectiveness, evaluation of immunotherapeutic drugs must also make allowance for immune-related response patterns.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

An “immunomodulator” or “immunoregulator” refers to an agent, e.g., an agent targeting a component of a signaling pathway that can be involved in modulating, regulating, or modifying an immune response. “Modulating,” “regulating,” or “modifying” an immune response refers to any alteration in a cell of the immune system or in the activity of such cell (e.g., an effector T cell, such as a Th1 cell). Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified, some of which can have enhanced function in a tumor microenvironment. In some embodiments, the immunomodulator targets a molecule on the surface of a T cell. An “immunomodulatory target” or “immunoregulatory target” is a molecule, e.g., a cell surface molecule, that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying the immune system or an immune response. In certain embodiments, the immunotherapy comprises administering an antibody to a subject. In other embodiments, the immunotherapy comprises administering a small molecule to a subject. In other embodiments, the immunotherapy comprises administering a cytokine or an analog, variant, or fragment thereof.

“Immuno stimulating therapy” or “immuno stimulatory therapy” refers to a therapy that results in increasing (inducing or enhancing) an immune response in a subject for, e.g., treating cancer.

“Potentiating an endogenous immune response” means increasing the effectiveness or potency of an existing immune response in a subject. This increase in effectiveness and potency can be achieved, for example, by overcoming mechanisms that suppress the endogenous host immune response or by stimulating mechanisms that enhance the endogenous host immune response.

A therapeutically effective amount of a drug includes a “prophylactically effective amount,” which is any amount of the drug that, when administered alone or in combination with an anti-neoplastic agent to a subject at risk of developing a cancer (e.g., a subject having a pre-malignant condition) or of suffering a recurrence of cancer, inhibits the development or recurrence of the cancer. In preferred embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

The term “tumor mutation burden” (TMB) as used herein refers to the number of somatic mutations in a tumor's genome and/or the number of somatic mutations per area of the tumor's genome. Germline (inherited) variants are excluded when determining TMB, because the immune system has a higher likelihood of recognizing these as self. Tumor mutation burden (TMB) can also be used interchangeably with “tumor mutation load,” “tumor mutational burden,” or “tumor mutational load.”

TMB is a genetic analysis of a tumor's genome and, thus, can be measured by applying sequencing methods well known to those of skill in the art. The tumor DNA can be compared with DNA from patient-matched normal tissue to eliminate germline mutations or polymorphisms.

In some embodiments, TMB is determined by sequencing tumor DNA using a high-throughput sequence technique, e.g., next-generation sequencing (NGS) or an NGS-based method. In some embodiments, the NGS-based method is selected from whole genome sequencing (WGS), whole exome sequencing (WES), or comprehensive genomic profiling (CGP) of cancer gene panels such as FOUNDATIONONE® CDX™ and MSK-IMPACT clinical tests. In some embodiments, TMB, as used herein, refers to the number of somatic mutations per megabase (Mb) of DNA sequenced. In one embodiment, TMB is measured using the total number of nonsynonymous mutations, e.g., missense mutation (i.e., changing a particular amino acid in the protein) and/or nonsense (causing premature termination and thus truncation of the protein sequence), identified by normalizing matched tumor with germline samples to exclude any inherited germline genetic alterations. In another embodiment, TMB is measured using the total number of missense mutations in a tumor. In order to measure TMB, a sufficient amount of sample is required. In one embodiment, tissue sample (for example, a minimum of 10 slides) is used for evaluation. In some embodiments, TMB is expressed as NsMs per megabase (NsM/Mb). 1 megabase represents 1 million bases.

The TMB status can be a numerical value or a relative value, e.g., high, medium, or low; within the highest fractile, or within the top tertile, of a reference set.

The term “high TMB” as used herein refers to a number of somatic mutations in a tumor's genome that is above a number of somatic mutations that is normal or average. In some embodiments, a TMB has a score of at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, or at least 500; in other embodiments a high TMB has a score of at least at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, or at least 250; and, in a particular embodiment, a high TMB has a score of at least 243. In other embodiments, a “high TMB” refers to a TMB within the highest fractile of the reference TMB value. For example, all subject's with evaluable TMB data are grouped according to fractile distribution of TMB, i.e., subjects are rank ordered from highest to lowest number of genetic alterations and divided into a defined number of groups. In one embodiment, all subjects with evaluable TMB data are rank ordered and divided into thirds and a “high TMB” is within the top tertile of the reference TMB value. In a particular embodiment, the tertile boundaries are 0<100 genetic alterations; 100 to 243 genetic alterations; and >243 genetic alterations. It should be understood that, once rank ordered, subjects with evaluable TMB data can be divided into any number of groups, e.g., quartiles, quintiles, etc. In some embodiments, a “high TMB” refers to a TMB of at least about 20 mutations/tumor, at least about 25 mutations/tumor, at least about 30 mutations/tumor, at least about 35 mutations/tumor, at least about 40 mutations/tumor, at least about 45 mutations/tumor, at least about 50 mutations/tumor, at least about 55 mutations/tumor, at least about 60 mutations/tumor, at least about 65 mutations/tumor, at least about 70 mutations/tumor, at least about 75 mutations/tumor, at least about 80 mutations/tumor, at least about 85 mutations/tumor, at least about 90 mutations/tumor, at least about 95 mutations/tumor, or at least about 100 mutations/tumor. In some embodiments, a “high TMB” refers to a TMB of at least about 105 mutations/tumor, at least about 110 mutations/tumor, at least about 115 mutations/tumor, at least about 120 mutations/tumor, at least about 125 mutations/tumor, at least about 130 mutations/tumor, at least about 135 mutations/tumor, at least about 140 mutations/tumor, at least about 145 mutations/tumor, at least about 150 mutations/tumor, at least about 175 mutations/tumor, or at least about 200 mutations/tumor. In certain embodiments, a tumor having a high TMB has at least about 100 mutations/tumor.

The “high TMB” can also be referred to as the number of mutations per megabase of genome sequenced, e.g., as measured by a mutation assay, e.g., FOUNDATIONONE® CDX™ assay. In one embodiment, the high TMB refers to at least about 9, at least about 10, at least about 11, at least 12, at least about 13, at least about 14, at least about 15, at least about 16, at least about 17, at least about 18, at least about 19, or at least about 20 mutations per megabase of genome as measured by a FOUNDATIONONE® CDX™ assay. In a particular embodiment, the “high TMB” refers to at least 10 mutations per megabase of genome sequenced by a FOUNDATIONONE® CDX™ assay.

As used herein, the term “medium TMB” refers to a number of somatic mutations in a tumor's genome that is at or around a number of somatic mutations that is normal or average and the term “low TMB” refers to a number of somatic mutations in a tumor's genome that is below a number of somatic mutations that is normal or average. In a particular embodiment, a “high TMB” has a score of at least 243, a “medium TMB” has a score of between 100 and 242, and a “low TMB” has a score of less than 100 (or between 0 and 100). The “medium or low TMB” refers to less than 9 mutations per megabase of genome sequenced, e.g., as measured by a FOUNDATIONONE® CDX™ assay.

The term “reference TMB value” as referred to herein can be the TMB value shown in Table 9.

In some embodiments, TMB status can correlate with smoking status. In particular, subjects who currently or formerly smoke (d) often have more genetic alterations, e.g., missense mutations, than subjects who never smoke (d).

A tumor with a high TMB can also have a high neoantigen load. As used herein, the term “neoantigen” refers to a newly formed antigen that has not been previously recognized by the immune system. A neoantigen can be a protein or peptide that is recognized as foreign (or non-self) by the immune system. Transcription of a gene in the tumor genome harboring a somatic mutation results in mutated mRNA that, when translated, gives rise to a mutated protein, which is then processed and transported to the ER lumen and binds to MHC class I complex, facilitating T-cell recognition of the neoantigen. Neoantigen recognition can promote T-cell activation, clonal expansion, and differentiation into effector and memory T-cells. Neoantigen load can correlate with TMB. In some embodiments, TMB is assessed as a surrogate for measuring tumor neoantigen load. The TMB status of a tumor can be used as a factor, alone or in combination with other factors, in determining whether a patient is likely to benefit from a particular anti-cancer agent or type of treatment or therapy, e.g., immuno-oncology agents, e.g., an anti-PD-1 antibody or antigen-binding portion thereof or an anti-PD-L1 antibody or antigen-binding portion thereof. In one embodiment, a high TMB status (or a high TMB) indicates an enhanced likelihood of benefit from immuno-oncology and, thus, can be used to identify patients more likely to benefit from therapy of an anti-PD-1 antibody or antigen-binding portion thereof. Similarly, tumors with high tumor neoantigen load and high TMB are more likely to be immunogenic than tumors with low neoantigen load and low TMB. In addition, high-neoantigen/high-TMB tumors are more likely to be recognized as non-self by the immune system, thus triggering an immune-mediated antitumor response. In one embodiment, a high TMB status and a high neoantigen load indicate an enhanced likelihood of benefit from immuno-oncology, e.g., with an immunotherapy. As used herein, the term “benefit from therapy” refers to an improvement in one or more of overall survival, progression-free survival, partial response, complete response, and overall response rate and can also include a reduction in tumor growth or size, a decrease in severity of disease symptoms, an increase in frequency and duration of disease symptom-free periods, or a prevention of impairment or disability due to the disease affliction.

Other factors, e.g., environmental factors, can associate with TMB status. For example, smoking status of patients with NSCLC was correlated with TMB distribution, whereby current and former smokers had higher median TMB compared with those patients who had never smoked. See Peters et al., AACR, Apr. 1-5, 2017, Washington, D.C. The presence of a driver mutation in NSCLC tumors was associated with younger age, female sex, and non-smoker status. See Singal et al., ASCO, Jun. 1-5, 2017; Chicago, IL. A trend associating the presence of driver mutations, such as EGFR, ALK, or KRAS, with lower TMB was observed (P=0.06). Davis et al., AACR, Apr. 1-5, 2017, Washington, D.C.

The term “somatic mutation” as used herein refers to an acquired alteration in DNA that occurs after conception. Somatic mutations can occur in any of the cells of the body except the germ cells (sperm and egg) and therefore are not passed on to children. These alterations can, but do not always, cause cancer or other diseases. The term “germline mutation” refers to a gene change in a body's reproductive cell (egg or sperm) that becomes incorporated into the DNA of every cell in the body of the offspring. Germline mutations are passed on from parents to offspring. Also called a “hereditary mutation.” In the analysis of TMB, germline mutations are considered as a “baseline,” and are subtracted from the number of mutations found in the tumor biopsy to determine the TMB within the tumor. As germline mutations are found in every cell in the body, their presence can be determined via less invasive sample collections than tumor biopsies, such as blood or saliva. Germline mutations can increase the risk of developing certain cancers, and can play a role in the response to chemotherapy.

The term “measuring” or “measured” or “measurement” when referring to TMB status means determining a measurable quantity of somatic mutations in a biological sample of the subject. It will be appreciated that measuring can be performed by sequencing nucleic acids, e.g., cDNA, mRNA, exoRNA, ctDNA, and cfDNA, in the sample. The measuring is performed on a subject's sample and/or a reference sample or samples and can, for example, be detected de novo or correspond to a previous determination. The measuring can be performed, for example, using PCR methods, qPCR methods, Sanger sequencing methods, genomic profiling methods (including comprehensive gene panels), exome sequencing methods, genome sequencing methods, and/or any other method disclosed herein, as is known to a person of skill in the art. In some embodiments, the measuring identifies a genomic alteration in the sequenced nucleic acids. The genomic (or gene) profiling methods can involve panels of a predetermined set of genes, e.g., 150-500 genes, and in some instances the genomic alterations evaluated in the panel of genes are correlated with total somatic mutations evaluated.

The term “genomic alteration” as used herein refers to a change (or mutation) in the nucleotide sequence of the genome of a tumor, which change is not present in the germline nucleotide sequence, and which in some embodiments is a nonsynonymous mutation including, but not limited to, a base pair substitution, a base pair insertion, a base pair deletion, a copy number alteration (CNA), a gene rearrangement, and any combination thereof. In a particular embodiment, the genomic alterations measured in the biological sample are missense mutations.

The term “whole genome sequencing” or “WGS,” as used herein, refers to a method of sequencing the entire genome. The term “whole exome sequencing” or “WES,” as used herein, refers to a method of sequencing all the protein-coding regions (exons) of the genome.

A “cancer gene panel,” “hereditary cancer panel,” “comprehensive cancer panel,” or “multigene cancer panel,” as used herein, refers to a method of sequencing a subset of targeted cancer genes. In some embodiments, the CGP comprises sequencing at least about 15, at least about 20, at least about 25, at least about 30, at least about 35, at least about 40, at least about 45, or at least about 50 targeted cancer genes.

The term “genomic profiling assay,” “comprehensive genomic profiling,” or “CGP” refers to an assay that analyzes a panel of genes and selects introns for in vitro diagnosis. CGP is a combination of NGS and targeted bioinformatics analysis to screen for mutations in known clinically relevant cancer genes. This method can be used to catch mutations that are missed by testing “hotspots” (e.g., BRCA1/BRCA2 mutations or microsatellite markers). In one embodiment, the genes in the panel are cancer-related genes. In another embodiment, a genomic profiling assay is a FOUNDATIONONE® assay.

The term “harmonization” refers to a study conducted to determine the comparability between two or more measures and/or diagnostic tests. Harmonization studies provide a systematic approach to address questions of how diagnostic tests compare with each other, as well as their interchangeability when used to determine the biomarker status of a patient's tumor. In general, at least one well-characterized measure and/or diagnostic test is used as a standard for comparison with others. Concordance assessment is often utilized in harmonization studies.

The term “concordance,” as used herein, refers to a degree of agreement between two measurements and/or diagnostic tests. Concordance can be established using both qualitative and quantitative methods. Quantitative methods to assess concordance differ based on the type of measurement. A particular measurement can be expressed either as 1) a categorical/dichotomized variable or 2) a continuous variable. A “categorical/dichotomized variable” (e.g., above or below TMB cut-off) may use percent agreements, such as overall percent agreement (OPA), positive percent agreement (PPA), or negative percent agreement (NPA), to assess concordance. A “continuous variable” (e.g., TMB by WES) uses Spearman's rank correlation or Pearson's correlation coefficient (r), which takes on values −1≤r≤+1, to assess concordance across a spectrum of values (Note r=+1 or −1 means that each of the variables is perfectly correlated). The term “analytical concordance” refers to the degree of agreement in the performance (e.g., identification of biomarkers, genomic alteration types, and genomic signatures, and assessment of test reproducibility) of two assays or diagnostic tests to support clinical use. The term “clinical concordance” refers to the degree of agreement in how the two assays or diagnostic tests correlate with clinical outcome.

The term “microsatellite instability” or “MSI” refers to a change that occurs in the DNA of certain cells (such as tumor cells) in which the number of repeats of microsatellites (short, repeated sequences of DNA) is different than the number of repeats that was in the DNA when it was inherited. MSI can be high microsatellite instability (MSI-H) or low microsatellite instability (MSI-L). Microsatellites are short tandem DNA repeat sequences of 1-6 bases. These are prone to DNA replication errors, which are repaired by mismatch repair (MMR). Hence microsatellites are good indicators of genome instability, especially deficient mismatch repair (dMMR). MSI is usually diagnosed by screening 5 microsatellite markers (BAT-25, BAT-26, NR21, NR24, and NR27). MSI-H represents the presence of at least 2 unstable markers among 5 microsatellite markers analyzed (or ≥30% of the markers if a larger panel is used). MSI-L means instability of 1 MSI marker (or 10%-30% of markers in larger panels). MSS means the absence of an unstable microsatellite marker.

The term “biological sample” as used herein refers to biological material isolated from a subject. The biological sample can contain any biological material suitable for determining TMB, for example, by sequencing nucleic acids in the tumor (or circulating tumor cells) and identifying a genomic alteration in the sequenced nucleic acids. The biological sample can be any suitable biological tissue or fluid such as, for example, tumor tissue, blood, blood plasma, and serum. In one embodiment, the sample is a tumor tissue biopsy, e.g., a formalin-fixed, paraffin-embedded (FFPE) tumor tissue or a fresh-frozen tumor tissue or the like. In another embodiment, the biological sample is a liquid biopsy that, in some embodiments, comprises one or more of blood, serum, plasma, circulating tumor cells, exoRNA, ctDNA, and cfDNA.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±three days, i.e., every eleven days to every seventeen days. Similar approximations apply, for example, to once about every three weeks, once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

A list of abbreviations is provided in Table 1.

TABLE 1
List of Abbreviations

| Term | Definition |
|---|---|
| Ab | antibody |
| AE | adverse event |
| ALK | anaplastic lymphoma kinase |
| AUC | area under the concentration-time curve |
| BICR | blinded independent central review |
| BMS | Bristol-Myers Squibb |
| BSA | body surface area |
| cfDNA | cell-free DNA |
| CI | confidence interval |
| CNS | central nervous system |
| CONSORT | consolidated standards of reporting trials |
| CR | complete response |
| ctDNA | circulating tumor DNA |
| CTLA-4 | cytotoxic T-lymphocyte-associated protein 4 |
| ECOG | Eastern Cooperative Oncology Group |
| e.g. | exempli gratia (for example) |
| EGFR | epidermal growth factor receptor |
| ELISA | enzyme-linked immunosorbent assay |
| exoRNA | exosomal RNA |
| HuMab | human antibody; human monoclonal antibody |
| i.e. | id est (that is) |
| IV | Intravenous |
| Kg | kilogram |
| mAb | monoclonal antibody |
| MB | megabase |
| mg | milligram |
| MO | month |
| N | number of subjects or observations |
| NCCN | National Comprehensive Cancer Network |
| NSCLC | non-small cell lung cancer |
| ORR | overall response rate |
| OS | overall survival |
| PD-1 | programmed death-1 |
| PD-L1 | programmed death-ligand 1 |
| PD-L2 | programmed death-ligand 2 |
| PFS | progression-free survival |
| PR | partial response |
| Q2W | once every two weeks |
| Q6W | once every six weeks |
| Q12W | once every twelve weeks |
| RCC | renal cell carcinoma |
| RECIST | response evaluation criteria in solid tumors |
| TILs | tumor infiltrating lymphocytes |
| TMB | tumor mutation burden |
| WES | whole exome sequencing |
| WGS | whole genome sequencing |

Various aspects of the disclosure are described in further detail in the following subsections.

### Methods of Tumor Mutation Burden (TMB) Measurement for Prediction and Prognosis

The present disclosure is directed to a method for identifying a subject suitable for treatment with immunotherapy, e.g., an anti-PD-1 antibody or antigen-binding portion thereof (“an anti-PD-1 antibody”) or an anti-PD-L1 antibody or antigen-binding portion thereof (“an anti-PD-L1 antibody”), comprising measuring a tumor mutational burden (TMB) status of a biological sample of the subject. The disclosure is based on the fact that different tumor types exhibit different levels of immunogenicity and that tumor immunogenicity is directly related to TMB and/or neoantigen load.

As a tumor grows, it accumulates somatic mutations not present in germline DNA. Tumor mutation burden (TMB) refers to the number of somatic mutations in a tumor's genome and/or the number of somatic mutations per area of the tumor genome (after taking into account germline variant DNA). The acquisition of somatic mutations and, thus, a higher TMB can be influenced by distinct mechanisms, such as exogenous mutagen exposure (e.g., tobacco smoking or UV light exposure) and DNA mismatch repair mutations (e.g., MSI in colorectal and esophageal cancers). In solid tumors, about 95% of mutations are single-base substitutions. (Vogelstein et al., Science (2013) 339:1546-1558.) A “nonsynonymous mutation” herein refers to a nucleotide mutation that alters the amino acid sequence of a protein. Missense mutations and nonsense mutations can be both nonsynonymous mutations. A “missense mutation” herein refers to a nonsynonymous point mutation in which a single nucleotide change results in a codon that codes for a different amino acid. A “nonsense mutation” herein refers to a nonsynonymous point mutation in which a codon is changed to a premature stop codon that leads to truncation of the resulting protein.

In one embodiment, somatic mutations can be expressed at the RNA and/or protein level, resulting in neoantigens (also referred to as neoepitopes). Neoantigens can influence an immune-mediated anti-tumor response. For example, neoantigen recognition can promote T-cell activation, clonal expansion, and differentiation into effector and memory T-cells.

As a tumor develops, early clonal mutations (or “trunk mutations”) can be carried by most or all tumor cells, while late mutations (or “branch mutations”) can occur in only a subset of tumor cells or regions. (Yap et al., Sci Tranl Med (2012) 4:1-5; Jamai-Hanjani et al., (2015) Clin Cancer Res 21:1258-1266.) As a result, neoantigens derived from clonal “trunk” mutations are more widespread in the tumor genome than “branch” mutations and, thus, can lead to a high number of T cells reactive against the clonal neoantigen. (McGranahan et al., (2016) 351:1463-1469.) Generally, tumors with a high TMB can also have a high neoantigen load, which can lead to high tumor immunogenicity and increased T-cell reactivity and anti-tumor response. As such, cancers with a high TMB can respond well to treatment with immunotherapies, e.g., an anti-PD-1 antibody or anti-PD-L1 antibody.

Advances in sequencing technologies allow for evaluation of the tumor's genomic mutation landscape. Any sequencing methods known to those of skill in the art can be used to sequence nucleic acids from the tumor genome (e.g., obtained from a biological sample from a subject afflicted with a tumor). In one embodiment, PCR or qPCR methods, Sanger sequencing methods, or next-generation sequencing (“NGS”) methods (such as genomic profiling, exome sequencing, or genome sequencing) can be used to measure TMB. In some embodiments, the TMB status is measured using genomic profiling. Genomic profiling involves analyzing nucleic acids from tumor samples, including coding and non-coding regions, and can be performed using methods having integrated optimized nucleic acid selection, read alignment, and mutation calling. In some embodiments, gene profiling provides next generation sequencing (NGS)-based analysis of tumors that can be optimized on a cancer-by-cancer, gene-by-gene, and/or site-by-site basis. Genome profiling can integrate the use of multiple, individually tuned, alignment methods or algorithms to optimize performance in sequencing methods, particularly in methods that rely on massively parallel sequencing of a large number of diverse genetic events in a large number of diverse genes. Genomic profiling provides for a comprehensive analysis of a subject's cancer genome, with clinical grade quality, and the output of the genetic analysis can be contextualized with relevant scientific and medical knowledge to increase the quality and efficiency of cancer therapy.

Genomic profiling involves a panel of a predefined set of genes comprising as few as five genes or as many as 1000 genes, about 25 genes to about 750 genes, about 100 genes to about 800 genes, about 150 genes to about 500 genes, about 200 genes to about 400 genes, about 250 genes to about 350 genes. In one embodiment, the genomic profile comprises at least 300 genes, at least 305 genes, at least 310 genes, at least 315 genes, at least 320 genes, at least 325 genes, at least 330 genes, at least 335 genes, at least 340 genes, at least 345 genes, at least 350 genes, at least 355 genes, at least 360 genes, at least 365 genes, at least 370 genes, at least 375 genes, at least 380 genes, at least 385 genes, at least 390 genes, at least 395 genes, or at least 400 genes. In another embodiment, the genomic profile comprises at least 325 genes. In a particular embodiment, the genomic profile comprises at least 315 cancer-related genes and introns in 28 genes (FOUNDATIONONE®) or the complete DNA coding sequence of 406 genes, introns in 31 genes with rearrangements, and the RNA sequence (cDNA) of 265 genes (FOUNDATIONONE® Heme). In another embodiment, the genomic profile comprises 26 genes and 1000 associated mutations (EXODX® Solid Tumor). In yet another embodiment, the genomic profile comprises 76 genes (Guardant360). In yet another embodiment, the genomic profile comprises 73 genes (Guardant360). In another embodiment, the genomic profile comprises 354 genes and introns in 28 genes for rearrangements (FOUNDATIONONE® CDX™). In certain embodiments, the genomic profile is FOUNDATIONONE® F1CDx. In another embodiment, the genomic profile comprises 468 genes (MSK-IMPACT™). One or more genes can be added to the genome profile as more genes are identified to be related to oncology.

### FOUNDATIONONE® Assay

The FOUNDATIONONE® assay is comprehensive genomic profiling assay for solid tumors, including but not limited to solid tumors of the lung, colon, and breast, melanoma, and ovarian cancer. The FOUNDATIONONE® assay uses a hybrid-capture, next-generation sequencing test to identify genomic alterations (base substitutions, insertions and deletions, copy number alterations, and rearrangements) and select genomic signatures (e.g., TMB and microsatellite instability). The assay covers 322 unique genes, including the entire coding region of 315 cancer-related genes, and selected introns from 28 genes. The full list of FOUNDATIONONE® assay genes is provided in Tables 2 and 3. See FOUNDATIONONE: Technical Specifications, Foundation Medicine, Inc., available at FoundationMedicine.com, last visited Mar. 16, 2018, which is incorporated by reference herein in its entirety.

TABLE 2
List of genes wherein entire coding sequences
are assayed in the FOUNDATIONONE ® assay.

ABL1
ABL2
ACVR1B
AKT1
AKT2
AKT3
ALK
AMER1
(FAM123B)
APC
AR
ARAF
ARFRP1
ARID1A
ARID1B
ARID2
ASXL1
ATM
ATR
ATRX
AURKA
AURKB
AXIN1
AXL
BAP1
BARD1
BCL2
BCL2L1
BCL2L2
BCL6
BCOR
BCORL1
BLM
BRAF
BRCA1
BRCA2
BRD4
BRIP1
BTG1
BTK
C11orf30
(EMSY)
CARD11
CBFB
CBL
CCND1
CCND2
CCND3
CCNE1
CD274
(PD-L1)
CD79A
CD79B
CDC73
CDH1
CDK12
CDK4
CDK6
CDK8
CDKN1A
CDKN1B
CDKN2A
CDKN2B
CDKN2C
CEBPA
CHD2
CHD4
CHEK1
CHEK2
CIC
CREBBP
CRKL
CRLF2
CSF1R
CTCF
CTNNA1
CTNNB1
CUL3
CYLD
DAXX
DDR2
DICER1
DNMT3A
DOT1L
EGFR
EP300
EPHA3
EPHA5
EPHA7
EPHB1
ERBB2
ERBB3
ERBB4
ERG
ERRFl1
ESR1
EZH2
FAM46C
FANCA
FANCC
FANCD2
FANCE
FANCF
FANCG
FANCL
FAS
FAT1
FBXW7
FGF10
FGF14
FGF19
FGF23
FGF3
FGF4
FGF6
FGFR1
FGFR2
FGFR3
FGFR4
FH
FLCN
FLT1
FLT3
FLT4
FOXL2
FOXP1
FRS2
FUBP1
GABRA6
GATA1
GATA2
GATA3
GATA4
GATA6
GID4
(C17orf39)
GLl1
GNA11
GNA13
GNAQ
GNAS
GPR124
GRIN2A
GRM3
GSK3B
H3F3A
HGF
HNF1A
HRAS
HSD3B1
HSP90AA1
IDH1
IDH2
IGF1R
IGF2
IKBKE
IKZF1
IL7R
INHBA
INPP4B
IRF2
IRF4
IRS2
JAK1
JAK2
JAK3
JUN
KAT6A
(MYST3)
KDM5A
KDM5C
KDM6A
KDR
KEAP1
KEL
KIT
KLHL6
KMT2A
(MLL)
KMT2C
(MLL3)
KMT2D
(MLL2)
KRAS
LMO1
LRP1B
LYN
LZTR1
MAGI2
MAP2K1
(MEK1)
MAP2K2
(MEK2)
MAP2K4
MAP3K1
MCL1
MDM2
MDM4
MED12
MEF2B
MEN1
MET
MITF
MLH1
MPL
MRE11A
MSH2
MSH6
MTOR
MUTYH
MYC
MYCL
(MYCL1)
MYCN
MYD88
NF1
NF2
NFE2L2
NFKBIA
NKX2-1
NOTCH1
NOTCH2
NOTCH3
NPM1
NRAS
NSD1
NTRK1
NTRK2
NTRK3
NUP93
PAK3
PALB2
PARK2
PAX5
PBRM1
PDCD1LG2
(PD-L2)
PDGFRA
PDGFRB
PDK1
PIK3C2B
PIK3CA
PIK3CB
PIK3CG
PIK3R1
PIK3R2
PLCG2
PMS2
POLD1
POLE
PPP2R1A
PRDM1
PREX2
PRKAR1A
PRKCI
PRKDC
PRSS8
PTCH1
PTEN
PTPN11
QKI
RAC1
RAD50
RAD51
RAF1
RANBP2
RARA
RB1
RBM10
RET
RICTOR
RNF43
ROS1
RPTOR
RUNX1
RUNX1T1
SDHA
SDHB
SDHC
SDHD
SETD2
SF3B1
SLIT2
SMAD2
SMAD3
SMAD4
SMARCA4
SMARCB1
SMO
SNCAIP
SOCS1
SOX10
SOX2
SOX9
SPEN
SPOP
SPTA1
SRC
STAG2
STAT3
STAT4
STK11
SUFU
SYK
TAF1
TBX3
TERC
TERT
(Promoter
only)
TET2
TGFBR2
TNFAIP3
TNFRSF14
TOP1
TOP2A
TP53
TSC1
TSC2
TSHR
U2AF1
VEGFA
VHL
WISP3
WT1
XPO1
ZBTB2
ZNF217
ZNF703

TABLE 3
List of genes wherein selected introns are
assayed in the FOUNDATIONONE ® assay.

ALK
BCL2
BCR
BRAF
BRCA1
BRCA2
BRD4
EGFR
ETV1
ETV4
ETV5
ETV6
FGFR1
FGFR2
FGFR3
KIT
MSH2
MYB
MYC
NOTCH2
NTRK1
NTRK2
PDGFRA
RAF1
RARA
RET
ROS1
TMPRSS2

### FOUNDATIONONE® Heme Assay

The FOUNDATIONONE® Heme assay is comprehensive genomic profiling assay for hematologic malignancies and sarcomas. The FOUNDATIONONE® Heme assay uses a hybrid-capture, next-generation sequencing test to identify genomic alterations (base substitutions, insertions and deletions, copy number alterations, and rearrangements). The assay analyzes the coding regions of 406 genes, selected introns of 31 genes, and the RNA sequences of 265 genes commonly rearranged in cancer. The full list of FOUNDATIONONE® Heme assay genes is provided in Tables 4, 5, and 6. See FOUNDATIONONE® HEME: Technical Specifications, Foundation Medicine, Inc., available at FoundationMedicine.com, last visited Mar. 16, 2018, which is incorporated by reference herein in its entirety.

TABLE 4
List of genes wherein entire coding sequences are
assayed in the FOUNDATIONONE ® Heme assay.

ABL1
ACTB
AKT1
AKT2
AKT3
ALK
AMER1
(FAM123B
or WTX)
APC
APH1A
AR
ARAF
ARFRP1
ARHGAP26
(GRAF)
ARID1A
ARID2
ASMTL
ASXL1
ATM
ATR
ATRX
AURKA
AURKB
AXIN1
AXL
B2M
BAP1
BARD1
BCL10
BCL11B
BCL2
BCL2L2
BCL6
BCL7A
BCOR
BCORL1
BIRC3
BLM
BRAF
BRCA1
BRCA2
BRD4
BRIP1
(BACH1)
BRSK1
BTG2
BTK
BTLA
C11orf30
(EMSY)
CAD
CALR
CARD11
CBFB
CBL
CCND1
CCND2
CCND3
CCNE1
CCT6B
CD22
CD274
(PD-L1)
CD36
CD58
CD70
CD79A
CD79B
CDC73
CDH1
CDK12
CDK4
CDK6
CDK8
CDKN1B
CDKN2A
CDKN2B
CDKN2C
CEBPA
CHD2
CHEK1
CHEK2
CIC
CIITA
CKS1B
CPS1
CREBBP
CRKL
CRLF2
CSF1R
CSF3R
CTCF
CTNNA1
CTNNB1
CUX1
CXCR4
DAXX
DDR2
DDX3X
DNM2
DNMT3A
DOT1L
DTX1
DUSP2
DUSP9
EBF1
ECT2L
EED
EGFR
ELP2
EP300
EPHA3
EPHA5
EPHA7
EPHB1
ERBB2
ERBB3
ERBB4
ERG
ESR1
ETS1
ETV6
EXOSC6
EZH2
FAF1
FAM46C
FANCA
FANCC
FANCD2
FANCE
FANCF
FANCG
FANCL
FAS
(TNFRSF6)
FBXO11
FBXO31
FBXW7
FGF10
FGF14
FGF19
FGF23
FGF3
FGF4
FGF6
FGFR1
FGFR2
FGFR3
FGFR4
FHIT
FLCN
FLT1
FLT3
FLT4
FLYWCH1
FOXL2
FOXO1
FOXO3
FOXP1
FRS2
GADD45B
GATA1
GATA2
GATA3
GID4
(C17orf39)
GNA11
GNA12
GNA13
GNAQ
GNAS
GPR124
GRIN2A
GSK3B
GTSE1
HDAC1
HDAC4
HDAC7
HGF
HIST1H1C
HIST1H1D
HIST1H1E
HIST1H2AC
HIST1H2AG
HIST1H2AL
HIST1H2AM
HIST1H2BC
HIST1H2BJ
HIST1H2BK
HIST1H2BO
HIST1H3B
HNF1A
HRAS
HSP90AA1
ICK
ID3
IDH1
IDH2
IGF1R
IKBKE
IKZF1
IKZF2
IKZF3
IL7R
INHBA
INPP4B
INPP5D
(SHIP)
IRF1
IRF4
IRF8
IRS2
JAK1
JAK2
JAK3
JARID2
JUN
KAT6A
(MYST3)
KDM2B
KDM4C
KDM5A
KDM5C
KDM6A
KDR
KEAP1
KIT
KLHL6
KMT2A
(MLL)
KMT2C
(MLL3)
KMT2D
(MLL2)
KRAS
LEF1
LRP1B
LRRK2
MAF
MAFB
MAGED1
MALT1
MAP2K1
(MEK1)
MAP2K2
(MEK2)
MAP2K4
MAP3K1
MAP3K14
MAP3K6
MAP3K7
MAPK1
MCL1
MDM2
MDM4
MED12
MEF2B
MEF2C
MEN1
MET
MIB1
MITF
MKI67
MLH1
MPL
MRE11A
MSH2
MSH3
MSH6
MTOR
MUTYH
MYC
MYCL
(MYCL1)
MYCN
MYD88
MYO18A
NCOR2
NCSTN
NF1
NF2
NFE2L2
NFKBIA
NKX2-1
NOD1
NOTCH1
NOTCH2
NPM1
NRAS
NT5C2
NTRK1
NTRK2
NTRK3
NUP93
NUP98
P2RY8
PAG1
PAK3
PALB2
PASK
PAX5
PBRM1
PC
PCBP1
PCLO
PDCD1
(PD-1)
PDCD11
PDCD1LG2
(PD-L2)
PDGFRA
PDGFRB
PDK1
PHF6
PIK3CA
PIK3CG
PIK3R1
PIK3R2
PIM1
PLCG2
POT1
PPP2R1A
PRDM1
PRKAR1A
PRKDC
PRSS8
PTCH1
PTEN
PTPN11
PTPN2
PTPN6
(SHP-1)
PTPRO
RAD21
RAD50
RAD51
RAF1
RARA
RASGEF1A
RB1
RELN
RET
RHOA
RICTOR
RNF43
ROS1
RPTOR
RUNX1
S1PR2
SDHA
SDHB
SDHC
SDHD
SERP2
SETBP1
SETD2
SF3B1
SGK1
SMAD2
SMAD4
SMARCA1
SMARCA4
SMARCB1
SMC1A
SMC3
SMO
SOCS1
SOCS2
SOCS3
SOX10
SOX2
SPEN
SPOP
SRC
SRSF2
STAG2
STAT3
STAT4
STAT5A
STAT5B
STAT6
STK11
SUFU
SUZ12
TAF1
TBL1XR1
TCF3
(E2A)
TCL1A
(TCL1)
TET2
TGFBR2
TLL2
TMEM30A
TMSB4XP8
(TMSL3)
TNFAIP3
TNFRSF11A
TNFRSF14
TNFRSF17
TOP1
TP53
TP63
TRAF2
TRAF3
TRAF5
TSC1
TSC2
TSHR
TUSC3
TYK2
U2AF1
U2AF2
VHL
WDR90
WHSC1
(MMSET
or NSD2)
WISP3
WT1
XBP1
XPO1
YY1AP1
ZMYM3
ZNF217
ZNF24
(ZSCAN3)
ZNF703
ZRSR2

TABLE 5
List of genes wherein selected introns are assayed
in the FOUNDATIONONE ® Heme assay.

ALK
BCL2
BCL6
BCR
BRAF
CCND1
CRLF2
EGFR
EPOR
ETV1
ETV4
ETV5
ETV6
EWSR1
FGFR2
IGH
IGK
IGL
JAK1
JAK2
KMT2A (MLL)
MYC
NTRK1
PDGFRA
PDGFRB
RAF1
RARA
RET
ROS1
TMPRSS2
TRG

TABLE 6
List of genes wherein RNA sequences are assayed in
the FOLTNDATIONONE ® Heme assay.

ABI1
ABL1
ABL2
ACSL6
AFF1
AFF4
ALK
ARHGAP26
(GRAF)
ARHGEF12
ARLD1A
ARNT
ASXL1
ATF1
ATG5
ATIC
BCL10
BCL11A
BCL11B
BCL2
BCL3
BCL6
BCL7A
BCL9
BCOR
BCR
BIRC3
BRAF
BTG1
CAMTA1
CARS
CBFA2T3
CBFB
CBL
CCND1
CCND2
CCND3
CD274
(PD-L1)
CDK6
CDX2
CHIC2
CHN1
CIC
CIITA
CLP1
CLTC
CLTCL1
CNTRL
(CEP110)
COL1A1
CREB3L1
CREB3L2
CREBBP
CRLF2
CSF1
CTNNB1
DDIT3
DDX10
DDX6
DEK
DUSP22
EGFR
EIF4A2
ELF4
ELL
ELN
EML4
EP300
EPOR
EPS15
ERBB2
ERG
ETS1
ETV1
ETV4
ETV5
ETV6
EWSR1
FCGR2B
FCRL4
FEV
FGFR1
FGFR1OP
FGFR2
FGFR3
FLI1
FNBP1
FOXO1
FOXO3
FOXO4
FOXP1
FSTL3
FUS
GAS7
GLI1
GMPS
GPHN
HERPUD1
HEY1
HIP1
HIST1H4l
HLF
HMGA1
HMGA2
HOXA11
HOXA13
HOXA3
HOXA9
HOXC11
HOXC13
HOXD11
HOXD13
HSP90AA1
HSP90AB1
IGH
IGK
IGL
IKZF1
IL21R
IL3
IRF4
ITK
JAK1
JAK2
JAK3
JAZF1
KAT6A
(MYST3)
KDSR
KIF5B
KMT2A
(MLL)
LASP1
LCP1
LMO1
LMO2
LPP
LYL1
MAF
MAFB
MALT1
MDS2
MECOM
MKL1
MLF1
MLLT1
(ENL)
MLLT10
(AF10)
MLLT3
MLLT4
(AF6)
MLLT6
MN1
MNX1
MSI2
MSN
MUC1
MYB
MYC
MYH11
MYH9
NACA
NBEAP1
(BCL8)
NCOA2
NDRG1
NF1
NF2
NFKB2
NIN
NOTCH1
NPM1
NR4A3
NSD1
NTRK1
NTRK2
NTRK3
NUMA1
NUP214
NUP98
NUTM2A
OMD
P2RY8
PAFAH1B2
PAX3
PAX5
PAX7
PBX1
PCM1
PCSK7
PDCD1LG2
(PD-L2)
PDE4DIP
PDGFB
PDGFRA
PDGFRB
PER1
PHF1
PICALM
PIM1
PLAG1
PML
POU2AF1
PPP1CB
PRDM1
PRDM16
PRRX1
PSIP1
PTCH1
PTK7
RABEP1
RAF1
RALGDS
RAP1GDS1
RARA
RBM15
RET
RHOH
RNF213
ROS1
RPL22
RPN1
RUNX1
RUNX1T1
(ETO)
RUNX2
SEC31A
SEPT5
SEPT6
SEPT9
SET
SH3GL1
SLC1A2
SNX29
(RUNDC2A)
SRSF3
SS18
SSX1
SSX2
SSX4
STAT6
STL
SYK
TAF15
TAL1
TAL2
TBL1XR1
TCF3
(E2A)
TCL1A
(TCL1)
TEC
TET1
TFE3
TFG
TFPT
TFRC
TLX1
TLX3
TMPRSS2
TNFRSF11A
TOP1
TP63
TPM3
TPM4
TRIM24
TRIP11
TTL
TYK2
USP6
WHSC1
(MMSET
or NSD2)
WHSC1L1
YPEL5
ZBTB16
ZMYM2
ZNF384
ZNF521

### EXODX® Solid Tumor Assay

In one embodiment, TMB is measured using the EXODX® Solid Tumor assay. The EXODX® Solid Tumor assay is an exoRNA- and cfDNA-based assay, which detects actionable mutations in cancer pathways. The EXODX® Solid Tumor assay is a plasma-based assay that does not require a tissue sample. The EXODX® Solid Tumor assay covers 26 genes and 1000 mutations. The specific genes covered by the EXODX® Solid Tumor assay are shown in Table 7. See Plasma-Based Solid Tumor Mutation Panel Liquid Biopsy, Exosome Diagnostics, Inc., available at exosomedx.com, last accessed on Mar. 16, 2018.

TABLE 7
Genes covered by the
EXODX ® Solid Tumor assay.

BRAF
NRAS
PIK3CA
MEK1
KRAS
EGFR
KIT
PDGFRA
EML4-LK
ROS1
RET
HER-2/NEU;
ERBB2
ALK
AKT1
ARv7
PTEN
DH2
mTOR
TP53
NOTCH1
Hedgehog
FGFR3
NTRK1
TSC1
TSC2
CDKN2A

### Guardant360 Assay

In some embodiments, TMB status is determined using the Guardant360 assay. The Guardant360 assay measures mutations in at least 73 genes (Table 8), 23 indels (Table 9), 18 CNVs (Table 10), and 6 fusion genes (Table 11). See GuardantHealth.com, last accessed on Mar. 16, 2018.

TABLE 8
Guardant360 assay genes.

AKT1
ALK
APC
AR
ARAF
ARID1A
ATM
BRAF
BRCA1
BRCA2
CCND1
CCND2
CCNE1
CDH1
CDK4
CDK6
CDKN2A
CTNNB1
DDR2
EGFR
ERBB2
ESR1
EZH2
FBXW7
FGFR1
FGFR2
FGFR3
GATA3
GNA11
GNAQ
GNAS
HNF1A
HRAS
IDH1
IDH2
JAK2
JAK3
KIT
KRAS
MAP2K1
MAP2K2
MAPK1
MAPK3
MET
MLH1
MPL
MTOR
MYC
NF1
NFE2L2
NOTCH1
NPM1
NRAS
NTRK1
NTRK3
PDGFRA
PIK3CA
PTEN
PTPN11
RAF1
RB1
RET
RHEB
RHOA
RIT1
ROS1
SMAD4
SMO
STK11
TERT
(including
promoter)
TP53
TSC1
VHL

TABLE 9
Guardant360 assay indels.

APC
ARID1A
ATM
BRCA1
BRCA2
CDH1
CDKN2A
EGFR
ERBB2
GATA3
KIT
MET
MLH1
MTOR
NF1
PDGFRA
PTEN
RB1
SMAD4
STK11
TP53
TSC1
VHL

TABLE 10
Guardant360 assay amplifications (CNVs).

| | | | | | |
|---|---|---|---|---|---|
| AR | CCND2 | CDK6 | FGFR1 | KRAS | PDGFRA |
| BRAF | CCNE1 | EGFR | FGFR2 | MET | PIK3CA |
| CCND1 | CDK4 | ERBB2 | KIT | MYC | RAF1 |

TABLE 11
Guardant360 assay fusions.

| | | |
|---|---|---|
| ALK | FGFR3 | RET |
| FGFR2 | NTRK1 | ROS1 |

### ILLUMINA® TruSight Assay

In some embodiments, TMB is determined using the TruSight Tumor 170 assay (ILLUMINA®). The TruSight Tumor 170 assay is a next-generation sequencing assay that covers 170 genes associated with common solid tumors, which simultaneously analyzes DNA and RNA. The TruSight Tumor 170 assay assesses fusions, splice variants, insertions/deletions, single nucleotide variants (SNVs), and amplifications. The TruSight Tumor 170 assay gene lists are shown in Tables 12-14.

TABLE 12
TruSight Tumor 170 assay genes (amplifications).

| | | | | | |
|---|---|---|---|---|---|
| AKT2 | CDK4 | FGF1 | FGF7 | LAMP1 | PDGFRB |
| ALK | CDK6 | FGF10 | FGF8 | MDM2 | PIK3CA |
| AR | CHEK1 | FGF14 | FGF9 | MDM4 | PIK3CB |
| ATM | CHEK2 | FGF19 | FGFR1 | MET | PTEN |
| BRAF | EGFR | FGF2 | FGFR2 | MYC | RAF1 |
| BRCA1 | ERBB2 | FGF23 | FGFR3 | MYCL1 | RET |
| BRCA2 | ERBB3 | FGF3 | FGFR4 | MYCN | RICTOR |
| CCND1 | ERCC1 | FGF4 | JAK2 | NRAS | RPS6KB1 |
| CCND3 | ERCC2 | FGF5 | KIT | NRG1 | TFRC |
| CCNE1 | ESR1 | FGF6 | KRAS | PDGFRA | |

TABLE 13
TruSight Tumor 170 assay genes (fusions).

ABL1
AKT3
ALK
AR
AXL
BCL2
BRAF
BRCA1
BRCA2
CDK4
CSF1R
EGFR
EML4
ERBB2
ERG
ESR1
ETS1
ETV1
ETV4
ETV5
EWSR1
FGFR1
FGFR2
FGFR3
FGFR4
FLI1
FLT1
FLT3
JAK2
KDR
KIF5B
KIT
KMT2A (MLL)
MET
MLLT3
MSH2
MYC
NOTCH1
NOTCH2
NOTCH3
NRG1
NTRK1
NTRK2
NTRK3
PAX3
PAX7
PDGFRA
PDGFRB
PIK3CA
PPARG
RAF1
RET
ROS1
RPS6KB1
TMPRSS2

TABLE 14
TruSight Tumor 170 assay
genes (small variants).

AKT1
AKT2
AKT3
ALK
APC
AR
ARID1A
ATM
ATR
BAP1
BARD1
BCL2
BCL6
BRAF
BRCA1
BRCA2
BRIP1
BTK
CARD11
CCND1
CCND2
CCNE1
CD79A
CD79B
CDH1
CDK12
CDK4
CDK6
CDKN2A
CEBPA
CHEK1
CHEK2
CREBBP
CSF1R
CTNNB1
DDR2
DNMT3A
EGFR
EP300
ERBB2
ERBB3
ERBB4
ERCC1
ERCC2
ERG
ESR1
EZH2
FAM175A
FANCI
FANCL
FBXW7
FGF1
FGF10
FGF14
FGF2
FGF23
FGF3
FGF4
FGF5
FGF6
FGF7
FGF8
FGF9
FGFR1
FGFR2
FGFR3
FGFR4
FLT1
FLT3
FOXL2
GEN1
GNA11
GNAQ
GNAS
HNF1A
HRAS
IDH1
IDH2
INPP4B
JAK2
JAK3
KDR
KIT
KMT2A (MLL)
KRAS
MAP2K1
MAP2K2
MCL1
MDM2
MDM4
MET
MLH1
MLLT3
MPL
MRE11A
MSH2
MSH3
MSH6
MTOR
MUTYH
MYC
MYCL1
MYCN
MYD88
NBN
NF1
NOTCH1
NOTCH2
NOTCH3
NPM1
NRAS
NRG1
PALB2
PDGFRA
PDGFRB
PIK3CA
PIK3CB
PIK3CD
PIK3CG
PIK3R1
PMS2
PPP2R2A
PTCH1
PTEN
PTPN11
RAD51
RAD51B
RAD51C
RAD51D
RAD54L
RB1
RET
RICTOR
ROS1
RPS6KB1
SLX4
SMAD4
SMARCB1
SMO
SRC
STK11
TERT
TET2
TP53
TSC1
TSC2
VHL
XRCC2

### FOUNDATIONONE® F1CDx Assay

FOUNDATIONONE® CDX™ (“F1CDx”) is a next generation sequencing based in vitro diagnostic device for detection of substitutions, insertion and deletion alterations (indels), and copy number alterations (CNAs) in 324 genes and select gene rearrangements, as well as genomic signatures including microsatellite instability (MSI) and tumor mutation burden (TMB) using DNA isolated from formalin-fixed paraffin embedded (FFPE) tumor tissue specimens. F1CDx is approved by the United States Food and Drug Administration (FDA) for several tumor indications, including NSCLC, melanoma, breast cancer, colorectal cancer, and ovarian cancer.

The F1CDx assay employs a single DNA extraction method from routine FFPE biopsy or surgical resection specimens, 50-1000 ng of which will undergo whole-genome shotgun library construction and hybridization-based capture of all coding exons from 309 cancer-related genes, one promoter region, one non-coding (ncRNA), and selected intronic regions from 34 commonly rearranged genes, 21 of which also include the coding exons. Tables 15 and 16 provide the complete list of genes included in F1CDx. In total, the assay detects alterations in a total of 324 genes. Using the ILLUMINA® HiSeq 4000 platform, hybrid capture-selected libraries are sequenced to high uniform depth (targeting >500× median coverage with >99% of exons at coverage >100×). Sequence data is then processed using a customized analysis pipeline designed to detect all classes of genomic alterations, including base substitutions, indels, copy number alterations (amplifications and homozygous gene deletions), and selected genomic rearrangements (e.g., gene fusions). Additionally, genomic signatures including microsatellite instability (MSI) and tumor mutation burden (TMB) are reported.

TABLE 15
Genes with full coding exonic regions included in
FOUNDATIONONE ® CDX ™ for the
detection of substitutions, insertions and deletions
(indels), and copy number alterations (CNAs).

ABL1
ACVR1B
AKT1
AKT2
AKT3
ALK
ALOX12B
AMER1
APC
AR
ARAF
ARFRP1
ARID1A
ASXL1
ATM
ATR
ATRX
AURKA
AURKB
AXIN1
AXL
BAP1
BARD1
BCL2
BCL2L1
BCL2L2
BCL6
BCOR
BCORL1
BRAF
BRCA1
BRCA2
BRD4
BRIP1
BTG1
BTG2
BTK
C11orf30
CALR
CARD11
CASP8
CBFB
CBL
CCND1
CCND2
CCND3
CCNE1
CD22
CD274
CD70
CD79A
CD79B
CDC73
CDH1
CDK12
CDK4
CDK6
CDK8
CDKN1A
CDKN1B
CDKN2A
CDKN2B
CDKN2C
CEBPA
CHEK1
CHEK2
CIC
CREBBP
CRKL
CSF1R
CSF3R
CTCF
CTNNA1
CTNNB1
CUL3
CUL4A
CXCR4
CYP17A1
DAXX
DDR1
DDR2
DIS3
DNMT3A
DOT1L
EED
EGFR
EP300
EPHA3
EPHB1
EPHB4
ERBB2
ERBB3
ERBB4
ERCC4
ERG
ERRFI1
ESR1
EZH2
FAM46C
FANCA
FANCC
FANCG
FANCL
FAS
FBXW7
FGF10
FGF12
FGF14
FGF19
FGF23
FGF3
FGF4
FGF6
FGFR1
FGFR2
FGFR3
FGFR4
FH
FLCN
FLT1
FLT3
FOXL2
FUBP1
GABRA6
GATA3
GATA4
GATA6
GID4
(C17orf39)
GNA11
GNA13
GNAQ
GNAS
GRM3
GSK3B
H3F3A
HDAC1
HGF
HNF1A
HRAS
HSD3B1
ID3
IDH1
IDH2
IGF1R
IKBKE
IKZF1
INPP4B
IRF2
IRF4
IRS2
JAK1
JAK2
JAK3
JUN
KDM5A
KDM5C
KDM6A
KDR
KEAP1
KEL
KIT
KLHL6
KMT2A (MLL)
KMT2D (MLL2)
KRAS
LTK
LYN
MAF
MAP2K1
MAP2K2
MAP2K4
MAP3K1
MAP3K13
MAPK1
MCL1
MDM2
MDM4
MED12
MEF2B
MEN1
MERTK
MET
MITF
MKNK1
MLH1
MPL
MRE11A
MSH2
MSH3
MSH6
MST1R
MTAP
MTOR
MUTYH
MYC
MYCL
MYCN
MYD88
NBN
NF1
NF2
NFE2L2
NFKBIA
NKX2-1
NOTCH1
NOTCH2
NOTCH3
NPM1
NRAS
NT5C2
NTRK1
NTRK2
NTRK3
P2RY8
PALB2
PARK2
PARP1
PARP2
PARP3
PAX5
PBRM1
PDCD1
PDCD1LG2
PDGFRA
PDGFRB
PDK1
PIK3C2B
PIK3C2G
PIK3CA
PIK3CB
PIK3R1
PIM1
PMS2
POLD1
POLE
PPARG
PPP2R1A
PPP2R2A
PRDM1
PRKAR1A
PRKCI
PTCH1
PTEN
PTPN11
PTPRO
QKI
RAC1
RAD21
RAD51
RAD51B
RAD51C
RAD51D
RAD52
RAD54L
RAF1
RARA
RB1
RBM10
REL
RET
RICTOR
RNF43
ROS1
RPTOR
SDHA
SDHB
SDHC
SDHD
SETD2
SF3B1
SGK1
SMAD2
SMAD4
SMARCA4
SMARCB1
SMO
SNCAIP
SOCS1
SOX2
SOX9
SPEN
SPOP
SRC
STAG2
STAT3
STK11
SUFU
SYK
TBX3
TEK
TET2
TGFBR2
TIPARP
TNFAIP3
TNFRSF14
TP53
TSC1
TSC2
TYRO3
U2AF1
VEGFA
VHL
WHSC1
WHSC1L1
WT1
XPO1
XRCC2
ZNF217
ZNF703

TABLE 16
Genes with selected intronic regions for the detection
of gene rearrangements, one with 3′UTR, one gene
with a promoter region and one ncRNA gene.

ALK introns 18, 19
BCL2 3′UTR
BCR introns 8, 13, 14
BRAF introns 7-10
BRCA1 introns 2, 7, 8, 12, 16, 19, 20
BRCA2 intron 2
CD74 introns 6-8
EGFR intron s7, 15, 24-27
ETV4 introns 5, 6
ETV5 introns 6, 7
ETV6 introns 5, 6
EWSR1 introns 7-13
EZR introns 9-11
FGFR1 intron 1, 5, 17
FGFR2 intron 1, 17
FGFR3 intron 17
KIT intron 16
KMT2A(MLL) introns 6-11
MSH2 intron 5
MYB intron 14
MYC intron 1
NOTCH2 intron 26
NTRK1 introns 8-10
NTRK2 Intron 12
NUTM1 intron 1
PDGFRA introns 7, 9, 11
RAF1 introns 4-8
RARA intron 2
RET introns 7-11
ROS1 introns 31-35
RSPO2 intron 1
SDC4 intron 2
SLC34A2 intron 4
TERC ncRNA
TERT Promoter
TMPRSS2 introns 1-3

The F1CDx assay identifies various alterations in the gene and/or intron sequences, including substitutions, insertions/deletions, and CNAs. The F1CDx assay was previously identifies as having concordance with an externally validated NGS assay and the FOUNDATIONONE® (F1 LDT) assay. See FOUNDATIONONE® CDX™: Technical Information, Foundation Medicine, Inc., available at FoundationMedicine.com, last visited Mar. 16, 2018, which is incorporated by reference herein in its entirety.

### MSK-IMPACT™

In some embodiments, TMB status is assessed using the MSK-IMPACT™ assay. The MSK-IMPACT™ assay uses next-generation sequencing to analyze the mutation status of 468 genes. Target genes are captured and sequenced on an ILLUMINA® HISEQ™ instrument. The MSK-IMPACT™ assay is approved by the US FDA for detection of somatic mutations and microsatellite instability in solid malignant neoplasms. The full list of 468 genes analyzed by the MSK-IMPACT™ assay is shown in Table 17. See Evaluation of Automatic Class III Designation for MSK-IMPACT (Integrated Mutation Profiling of Actionable Cancer Targets): Decision Summary, United States Food and Drug Administration, Nov. 15, 2017, available at accessdata.fda.gov.

TABLE 17
Genes analyzed by the MSK-IMPACT ™ assay.

ABL1
ACVR1
AGO2
AKT1
AKT2
AKT3
ALK
ALOX12B
AMER1
ANKRD11
APC
AR
ARAF
ARID1A
ARID1B
ARID2
ARID5B
ASXL1
ASXL2
ATM
ATR
ATRX
AURKA
AURKB
AXIN1
AXIN2
AXL
B2M
BABAM1
BAP1
BARD1
BBC3
BCL10
BCL2
BCL2L1
BCL2L11
BCL6
BCOR
BIRC3
BLM
BMPR1A
BRAF
BRCA1
BRCA2
BRD4
BRIP1
BTK
ABL1
CALR
CARD11
CARM1
CASP8
CBFB
CBL
CCND1
CCND2
CCND3
CCNE1
CD274
CD276
CD79A
CD79B
CDC42
CDC73
CDH1
CDK12
CDK4
CDK6
CDK8
CDKN1A
CDKN1B
CDKN2Ap14ARF
CDKN2Ap16INK4A
CDKN2B
CDKN2C
CEBPA
CENPA
CHEK1
CHEK2
CIC
CREBBP
CRKL
CRLF2
CSDE1
CSF1R
CSF3R
CTCF
CTLA-4
CTNNB1
CUL3
CXCR4
CYLD
CYSLTR2
DAXX
DCUN1D1
CALR
DDR2
DICER1
DIS3
DNAJB1
DNMT1
DNMT3A
DNMT3B
DOT1L
DROSHA
DUSP4
E2F3
EED
EGFL7
EGFR
EIF1AX
EIF4A2
EIF4E
ELF3
EP300
EPAS1
EPCAM
EPHA3
EPHA5
EPHA7
EPHB1
ERBB2
ERBB3
ERBB4
ERCC2
ERCC3
ERCC4
ERCC5
ERF
ERG
ERRFI1
ESR1
ETV1
ETV6
EZH1
EZH2
FAM175A
FAM46C
FAM58A
FANCA
FANCC
FAT1
FBXW7
DDR2
FGF19
FGF3
FGF4
FGFR1
FGFR2
FGFR3
FGFR4
FH
FLCN
FLT1
FLT3
FLT4
FOXA1
FOXL2
FOXO1
FOXP1
FUBP1
FYN
GATA1
GATA2
GATA3
GLI1
GNA11
GNAQ
GNAS
GPS2
GREM1
GRIN2A
GSK3B
H3F3A
H3F3B
H3F3C
HGF
HIST1H1C
HIST1H2BD
HIST1H3A
HIST1H3B
HIST1H3C
HIST1H3D
HIST1H3E
HIST1H3F
HIST1H3G
HIST1H3H
HIST1H3I
HIST1H3J
HIST2H3C
HIST2H3D
FGF19
HIST3H3
HLA-A
HLA-B
HNF1A
HOXB13
HRAS
ICOSLG
ID3
IDH1
IDH2
IFNGR1
IGF1
IGF1R
IGF2
IKBKE
IKZF1
IL10
IL7R
INHA
INHBA
INPP4A
INPP4B
INPPL1
INSR
IRF4
IRS1
IRS2
JAK1
JAK2
JAK3
JUN
KDM5A
KDM5C
KDM6A
KDR
KEAP1
KIT
KLF4
KMT2A
KMT2B
KMT2C
KMT2D
KNSTRN
KRAS
LATS1
LATS2
LMO1
HIST3H3
LYN
MALT1
MAP2K1
MAP2K2
MAP2K4
MAP3K1
MAP3K13
MAP3K14
MAPK1
MAPK3
MAPKAP1
MAX
MCL1
MDC1
MDM2
MDM4
MED12
MEF2B
MEN1
MET
MGA
MITF
MLH1
MPL
MRE11A
MSH2
MSH3
MSH6
MSI1
MSI2
MST1
MST1R
MTOR
MUTYH
MYC
MYCL1
MYCN
MYD88
MYOD1
NBN
NCOA3
NCOR1
NEGR1
NF1
NF2
NFE2L2
NFKBIA
LYN
NKX2-1
NKX3-1
NOTCH1
NOTCH2
NOTCH3
NOTCH4
NPM1
NRAS
NSD1
NTHL1
NTRK1
NTRK2
NTRK3
NUF2
NUP93
PAK1
PAK7
PALB2
PARK2
PARP1
PAX5
PBRM1
PDCD1
PDCD1LG2
PDGFRA
PDGFRB
PDPK1
PGR
PHOX2B
PIK3C2G
PIK3C3
PIK3CA
PIK3CB
PIK3CD
PIK3CG
PIK3R1
PIK3R2
PIK3R3
PIM1
PLCG2
PLK2
PMAIP1
PMS1
PMS2
PNRC1
POLD1
POLE
NKX2-1
PPARG
PPM1D
PPP2R1A
PPP4R2
PPP6C
PRDM1
PRDM14
PREX2
PRKAR1A
PRKCI
PRKD1
PTCH1
PTEN
PTP4A1
PTPN11
PTPRD
PTPRS
PTPRT
RAB35
RAC1
RAC2
RAD21
RAD50
RAD51
RAD51B
RAD51C
RAD51D
RAD52
RAD54L
RAF1
RARA
RASA1
RB1
RBM10
RECQL
RECQL4
REL
RET
RFWD2
RHEB
RHOA
RICTOR
RIT1
RNF43
ROS1
RPS6KA4
RPS6KB2
PPARG
RPTOR
RRAGC
RRAS
RRAS2
RTEL1
RUNX1
RXRA
RYBP
SDHA
SDHAF2
SDHB
SDHC
SDHD
SESN1
SESN2
SESN3
SETD2
SETD8
SF3B1
SH2B3
SH2D1A
SHOC2
SHQ1
SLX4
SMAD2
SMAD3
SMAD4
SMARCA4
SMARCB1
SMARCD1
SMO
SMYD3
SOCS1
SOS1
SOX17
SOX2
SOX9
SPEN
SPOP
SPRED1
SRC
SRSF2
STAG2
STAT3
STAT5A
STAT5B
STK11
RPTOR
STK19
STK40
SUFU
SUZ12
SYK
TAP1
TAP2
TBX3
TCEB1
TCF3
TCF7L2
TEK
TERT
TET1
TET2
TGFBR1
TGFBR2
TMEM127
TMPRSS2
TNFAIP3
TNFRSF14
TOP1
TP53
TP53BP1
TP63
TRAF2
TRAF7
TSC1
TSC2
TSHR
U2AF1
UPF1
VEGFA
VHL
VTCN1
WHSC1
WHSC1L1
WT1
WWTR1
XIAP
XPO1
XRCC2
YAP1
YES1
ZFHX3
STK19

### NEOGENOMICSR NEOTYPE™ Assays

In some embodiments, TMB is determined using a NEOGENOMICS® NEOTYOPE™ assay. In some embodiments, the TMB is determined using a NEOTYPE™ Discovery Profile. In some embodiments, the TMB is determined using a NEOTYPE™ Solid Tumor Profile. The NEOGENOMICS® assays measure the number of non-synonymous DNA coding sequence changes per megabase of sequenced DNA.

### ONCOMINE™ Tumor Mutation Load Assay

In some embodiments, TMB is determined using a THERMOFISHER SCIENTIFIC® ONCOMINE™ Tumor Mutation assay. In some embodiments, TMB is determined using a THERMOFISHER SCIENTIFIC® ION TORRENT™ ONCOMINE™ Tumor Mutation assay. The ION TORRENT™ ONCOMINE™ Tumor Mutation assay is a targeted NGS assay that quantitates somatic mutations to determine tumor mutation load. The assay covers 1.7 Mb of DNA.

### NOVOGENE™ NOVOPM™ Assay

In some embodiments, TMB is determined using a NOVOGENE™ NOVOPM™ assay. In some embodiments, TMB is determined using a NOVOGENE™ NOVOPM™ Cancer Panel assay. The NOVOGENE™ NOVOPM™ Cancer Panel assay is a comprehensive NGS cancer panel that analyzes the complete coding regions of 548 genes and the introns of 21 genes, representing about 1.5 Mb of DNA, and that are relevant for the diagnosis and/or treatment of solid tumors according to the National Comprehensive Cancer Network (NCCN) guidelines and medical literature. The assay detects SNV, InDel, fusion, and copy number variation (CNV) genomic abnormalities.

### Other TMB Assays

In some embodiments, TMB is determined using a TMB assay provided by CARIS® Life Sciences. In some embodiments, TMB is determined using the PESONALIS® ACE ImmunoID assay. In some embodiments, TMB is determined using the PGDX® CANCERXOME™-R assay.

In yet another particular embodiment, the genomic profiling detects all mutation types, i.e., single nucleotide variants, insertions/deletions (indels), copy number variations, and rearrangements, e.g., translocations, expression, and epigenetic markers.

Comprehensive gene panels often contain predetermined genes selected based on the type of tumor to be analyzed. Accordingly, the genomic profile used to measure TMB status can be selected based on the type of tumor the subject has. In one embodiment, the genomic profile can include a set of genes particular to a solid tumor. In another embodiment, the genomic profile can include a set of genes particular to hematologic malignancies and sarcomas.

In one embodiment, the genomic profile comprises one or more genes selected from the group consisting of ABL1, BRAF, CHEK1, FANCC, GATA3, JAK2, MITF, PDCD1LG2, RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCE, GID4 (C17orf39), KAT6A (MYST3), MRE11A, PDK1, RNF43, SYK, AKT2, BRIP1, CRKL, FANCG, GLI1, KDM5A, MSH2, PIK3C2B, ROS1, TAF1, AKT3, BTG1, CRLF2, FANCL, GNA1I, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf30 (EMSY), CTCF, FAT1, GNAQ, KDR, MUTYH, PIK3CG, RUNX1T1, TERT (promoter only), APC, CARD11, CTNNA1, FBXW7, GNAS, KEAP1, MYC, PIK3R1, SDHA, TET2, AR, CBFB, CTNNB1, FGF10, GPR124, KEL, MYCL (MYCL1), PIK3R2, SDHB, TGFBR2, ARAF, CBL, CUL3, FGF14, GRIN2A, KIT, MYCN, PLCG2, SDHC, TNFAIP3, ARFRP1, COND1, CYLD, FGF19, GRM3, KLHL6, MYD88, PMS2, SDHD, TNFRSF14, ARID1A, CCND2, DAXX, FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D) (MLL2), NFE2L2, PPP2R1A, SLIT2, TP53, ASXL1, CD274, DNMT3A, FGF6, HNF1A, KRAS, NFKB1A, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP90AA1, LYN, NOTCH2, PRKC1, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1, NRAS, PTCH1, SNCA1P, WISP3, AXL, CDK6, EPHB1, FLT1, IGF2, MAP2K2, NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, QK1, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFI1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, STAT3, and any combination thereof. In other embodiments, the TMB analysis further comprises identifying a genomic alteration in one or more of ETV4, TMPRSS2, ETV5, BCR, ETV1, ETV6, and MYB.

In another embodiment, the genomic profile comprises one or more genes selected from the group consisting of ABL1, 12B, ABL2, ACTB, ACVR1, ACVR1B, AGO2, AKT1, AKT2, AKT3, ALK, ALOX, ALOX12B, AMER1, AMER1 (FAM123B or WTX), AMER1 (FAM123B), ANKRD11, APC, APH1A, AR, ARAF, ARFRP1, ARHGAP26 (GRAF), ARID1A, ARID1B, ARID2, ARID5B, ARv7, ASMTL, ASXL1, ASXL2, ATM, ATR, ATRX, AURKA, AURKB, AXIN1, AXIN2, AXL, B2M, BABAM1, BAP1, BARD1, BBC3, BCL10, BCL11B, BCL2, BCL2L1, BCL2L11, BCL2L2, BCL6, BCL7A, BCOR, BCORL1, BIRC3, BLM, BMPR1A, BRAF, BRCA1, BRCA2, BRD4, BRIP1, BRIP1 (BACH1), BRSK1, BTG1, BTG2, BTK, BTLA, C11orf30 (EMSY), C11orf30, C11orf30 (EMSY), CAD, CALR, CARD11, CARMI, CASP8, CBFB, CBL, COND1, CCND2, COND3, CCNE1, CCT6B, CD22, CD274, CD274 (PD-L1), CD276, CD36, CD58, CD70, CD79A, CD79B, CDC42, CDC73, CDH1, CDK12, CDK4, CDK6, CDK8, CDKN1A, CDKN1B, CDKN2A, CDKN2Ap14ARF, CDKN2Ap16INK4A, CDKN2B, CDKN2C, CEBPA, CENPA, CHD2, CHD4, CHEK1, CHEK2, CIC, C11TA, CKS1B, CPS1, CREBBP, CRKL, CRLF2, CSDE1, CSF1R, CSF3R, CTCF, CTLA-4, CINN B1, CTNNA1, CTNNB1, CUL3, CUL4A, CUX1, CXCR4, CYLD, CYP17A1, CYSLTR2, DAXX, DCUN1D1, DDR1, DDR2, DDX3X, DH2, DICER1, DIS3, DNAJB1, DNM2, DNMT1, DNMT3A, DNMT3B, DOT1L, DROSHA, DTX1, DUSP2, DUSP4, DUSP9, E2F3, EBF1, ECT2L, EED, EGFL7, EGFR, EIF1AX, EIF4A2, EIF4E, ELF3, ELP2, EML4, EML4-ALK, EP300, EPAS1, EPCAM, EPHA3, EPHA5, EPHA7, EPHB1, EPHB4, ERBB2, ERBB3, ERBB4, ERCC1, ERCC2, ERCC3, ERCC4, ERCC5, ERF, ERG, ERRFI1, ERRFl1, ESR1, ETS1, ETV1, ETV4, ETV5, ETV6, EWSR1, EXOSC6, EZH1, EZH2, FAF1, FAM175A, FAM46C, FAM58A, FANCA, FANCC, FANCD2, FANCE, FANCE, FANCG, FANCI, FANCL, FAS, FAS (TNFRSF6), FAT1, FBXO11, FBXO31, FBXW7, FGF1, FGF10, FGF12, FGF14, FGF19, FGF2, FGF23, FGF3, FGF4, FGF5, FGF6, FGF7, FGF8, FGF9, FGFR1, FGFR2, FGFR3, FGFR4, FH, FHIT, FLON, FLII, FLT1, FLT3, FLT4, FLYWCH1, FOXA1, FOXL2, FOXO1, FOXO3, FOXP1, FRS2, FUBP1, FYN, GABRA6, GADD45B, GATA1, GATA2, GATA3, GATA4, GATA6, GEN1, GID4 ((17orf39), GID4 (C17orf39), GLI1, GLI1, GNA1I, GNA12, GNA13, GNAQ, GNAS, GPR124, GPS2, GREM1, GRIN2A, GRM3, GSK3B, GTSE1, H3F3A, H3F3B, H3F3C, HDAC1, HDAC4, HDAC7, Hedgehog, HER-2 NEU; ERBB2, HGF, HIST1H1C, HIST1H1D, HIST1H1E, HIST1H2AC, HIST1H2AG, HIST1H2AL, HIST1H2AM, HIST1H2BC, HIST1H2BD, HIST1H2BJ, HIST1H2BK, HIST1H2BO, HIST1H3A, HIST1H3B, HIST1H3C, HIST1H3D, HIST1H3E, HIST1H3F, HIST1H3G, HIST1H3H, HIST1H3I, HIST1H3J, HIST2H3C, HIST2H3D, HIST3H3, HLA-A, HLA-B, HNF1A, HOXB13, HRAS, HSD3B1, HSP90AA1, ICK, ICOSLG, ID3, IDH1, IDH2, IFNGR1, IGF1, IGF1R, IGF2, IKBKE, IKZF1, IKZF2, IKZF3, IL10, IL7R, INHA, INHBA, INPP4A, INPP4B, INPP5D (SHIP), INPPL1, INSR, IRF1, IRF2, IRF4, IRF8, IRS1, IRS2, JAK1, JAK2, JAK3, JARID2, JUN, K14, KAT6A (MYST3), KAT6A (MYST3), KDM2B, KDM4C, KDM5A, KDM5C, KDM6A, KDR, KEAP1, KEL, KIF5B, KIT, KLF4, KLHL6, KMT2A, KMT2A (MLL), KMT2B, KMT2C, KMT2 (MLL3), KMT2D, KMT2D) (MLL2), KNSTRN, KRAS, LAMP1, LATS1, LATS2, LEF1, LMO1, LRP1B, LRRK2, LTK, LYN, LZTR1, MAF, MAFB, MAGED1, MAGI2, MALT1, MAP2K1, MAP2K1 (MEK1), MAP2K2, MAP2K2 (MEK2), MAP2K4, MAP3, MAP3K1, MAP3K13, MAP3K14, MAP3K6, MAP3K7, MAPK1, MAPK3, MAPKAP1, MAX, MCL1, MDC1, MDM2, MDM4, MED12, MEF2B, MEF2C, MEK1, MEN1, MERTK, MET, MGA, MIB1, MITF, MKI67, MKNK1, MLH1, MLLT3, MPL, MRE11A, MRE11A, MSH2, MSH3, MSH6, MSI1, MSI2, MST1, MST1R, MTAP, MTOR, MUTYH, MYC, MYCL, MYCL (MYC L1), MYCL (MYCL1), MYCL1, MYCN, MYD88, MYO18A, MYOD1, NBN, NCOA3, NCOR1, NCOR2, NCSTN, NEGR1, NF1, NF2, NFE2L2, NFKB1A, NKX2-1, NKX3-1, NOD1, NOTCH1, NOTCH2, NOTCH3, NOTCH4, NPM1, NRAS, NRG1, NSD1, NT5C2, NTHL1, NTRK1, NTRK2, NTRK3, NUF2, NUP93, NUP98, P2RY8, PAG1, PAK1, PAK3, PAK7, PALB2, PARK2, PARP1, PARP2, PARP3, PASK, PAX3, PAX5, PAX7, PBRM1, PC, PCBP1, PCLO, PDCD1, PDCD1 (PD-1), PDCD11, PDCD1LG2, PDCD1LG2 (PD-L2), PDGFRA, PDGFRB, PDK1, PDPK1, PGR, PHF6, PHOX2B, PIK302B, PIK302G, PIK3C3, PIK3CA, PIK3CB, PIK3CD, PIK3CG, PIK3R1, PIK3R2, PIK3R3, PIM1, PLCG2, PLK2, PMAIP1, PMS1, PMS2, PNRC1, POLD1, POLE, POT1, PPARG, PPM1D, PPP2, PPP2R1A, PPP2R2A, PPP4R2, PPP6C, PRDM1, PRDM14, PREX2, PRKAR1A, PRKC1, PRKD1, PRKDC, PRSS8, PTCH1, PTEN, PTP4A1, PTPN11, PTPN2, PTPN6 (SHP-1), PTPRD, PTPRO, PTPRS, PTPRT, QK1, R1A, RAB35, RAC1, RAC2, RAD21, RAD50, RAD51, RAD51B, RAD51C, RAD51D, RAD52, RAD54L, RAF1, RANBP2, RARA, RASA1, RASGEF1A, RB1, RBM10, RECQL, RECQL4, REL, RELN, RET, RFWD2, RHEB, RHOA, RICTOR, RIT1, RNF43, ROS1, RPS6KA4, RPS6KB1, RPS6KB2, RPTOR, RRAGC, RRAS, RRAS2, RTEL1, RUNX1, RUNX1T1, RXRA, RYBP, SIPR2, SDHA, SDHAF2, SDHB, SDHC, SDHD, SERP2, SESN1, SESN2, SESN3, SETBP1, SETD2, SETD8, SF3B1, SGK1, SH2B3, SH2D1A, SHOC2, SHQ1, SLIT2, SLX4, SMAD2, SMAD3, SMAD4, SMARCA1, SMARCA4, SMARCB1, SMARCD1, SMC1A, SMC3, SMO, SMYD3, SNCA1P, SOCS1, SOCS2, SOCS3, SOS1, SOX10, SOX17, SOX2, SOX9, SPEN, SPOP, SPRED1, SPTA1, SRC, SRSF2, STAG2, STAT3, STAT4, STAT5A, STAT5B, STAT6, STK11, STK19, STK40, SUFU, SUZ12, SYK, TAF1, TAP1, TAP2, TBL1XR1, TBX3, TCEB1, TCF3, TCF3 (E2A), TCF7L2, TOL1A (TCL1), TEK, TERC, TERT, TERT Promoter, TET1, TET2, TFRC, TGFBR1, TGFBR2, TIPARP, TLL2, TMEM127, TMEM30A, TMPRSS2, TMSB4XP8 (TMSL3), TNFAIP3, TNFRSF11A, TNFRSF14, TNFRSF17, TOP1, TOP2A, TP53, TP53BP1, TP63, TRAF2, TRAF3, TRAF5, TRAF7, TSC1, TSC2, TSHR, TUSC3, TYK2, TYRO3, U2AF1, U2AF2, UPF1, VEGFA, VHL, VTCN1, WDR90, WHSC1, WHSC1 (MMSET or NSD2), WHSC1L1, WISP3, WT1, WWTR1, XBP1, XIAP, XPO1, XRCC2, YAP1, YES1, YY1AP1, ZBTB2, ZFHX3, ZMYM3, ZNF217, ZNF24 (ZSCAN3), ZNF703, ZRSR2, and any combination thereof.

In another embodiment, the genomic profiling assay comprises at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, or at least about 300 genes selected from the group consisting of ABL1, 12B, ABL2, ACTB, ACVR1, ACVR1B, AGO2, AKT1, AKT2, AKT3, ALK, ALOX, ALOX12B, AMER1, AMER1 (FAM123B or WTX), AMER1 (FAM123B), ANKRD11, APC, APH1A, AR, ARAF, ARFRP1, ARHGAP26 (GRAF), ARID1A, ARID1B, ARID2, ARID5B, ARv7, ASMTL, ASXL1, ASXL2, ATM, ATR, ATRX, AURKA, AURKB, AXIN1, AXIN2, AXL, B2M, BABAM1, BAP1, BARD1, BBC3, BCL10, BCL11B, BCL2, BCL2L1, BCL2L11, BCL2L2, BCL6, BCL7A, BCOR, BCORL1, BIRC3, BLM, BMPR1A, BRAF, BRCA1, BRCA2, BRD4, BRIP1, BRIP1 (BACH1), BRSK1, BTG1, BTG2, BTK, BTLA, C11orf30 (EMSY), C11orf30, C11orf30 (EMSY), CAD, CALR, CARD11, CARMI, CASP8, CBFB, CBL, COND1, COND2, CCND3, CCNE1, CCT6B, CD22, CD274, CD274 (PD-L1), CD276, CD36, CD58, CD70, CD79A, CD79B, CDC42, CDC73, CDH1, CDK12, CDK4, CDK6, CDK8, CDKN1A, CDKN1B, CDKN2A, CDKN2Ap14ARF, CDKN2Ap16INK4A, CDKN2B, CDKN2C, CEBPA, CENPA, CHD2, CHD4, CHEK1, CHEK2, CIC, CHITA, CKS1B, CPS1, CREBBP, CRKL, CRLF2, CSDE1, CSF1R, CSF3R, CTCF, CTLA-4, CTNN B1, CTNNA1, CTNNB1, CUL3, CUL4A, CUX1, CXCR4, CYLD, CYP17A1, CYSLTR2, DAXX, DCUN1D1, DDR1, DDR2, DDX3X, DH2, DICER1, DIS3, DNAJB1, DNM2, DNMT1, DNMT3A, DNMT3B, DOT1L, DROSHA, DTX1, DUSP2, DUSP4, DUSP9, E2F3, EBF1, ECT2L, EED, EGFL7, EGFR, EIF1AX, EIF4A2, EIF4E, ELF3, ELP2, EML4, EML4-ALK, EP300, EPAS1, EPCAM, EPHA3, EPHA5, EPHA7, EPHB1, EPHB4, ERBB2, ERBB3, ERBB4, ERCC1, ERCC2, ERCC3, ERCC4, ERCC5, ERF, ERG, ERRFI1, ERRFl1, ESR1, ETS1, ETV1, ETV4, ETV5, ETV6, EWSR1, EXOSC6, EZH1, EZH2, FAF1, FAM175A, FAM46C, FAM58A, FANCA, FANCC, FANCD2, FANCE, FANCE, FANCG, FANCI, FANCL, FAS, FAS (TNFRSF6), FAT1, FBXO11, FBXO31, FBXW7, FGF1, FGF10, FGF12, FGF14, FGF19, FGF2, FGF23, FGF3, FGF4, FGF5, FGF6, FGF7, FGF8, FGF9, FGFR1, FGFR2, FGFR3, FGFR4, FH, FHIT, FLON, FLII, FLT1, FLT3, FLT4, FLYWCH1, FOXA1, FOXL2, FOXO1, FOXO3, FOXP1, FRS2, FUBP1, FYN, GABRA6, GADD45B, GATA1, GATA2, GATA3, GATA4, GATA6, GEN1, GID4 ((17orf39), GID4 (C17orf39), GLI1, GLl1, GNA1I, GNA12, GNA13, GNAQ, GNAS, GPR124, GPS2, GREM1, GRIN2A, GRM3, GSK3B, GTSE1, H3F3A, H3F3B, H3F3C, HDAC1, HDAC4, HDAC7, Hedgehog, HER-2 NEU; ERBB2, HGF, HIST1H1C, HIST1H1D, HIST1H1E, HIST1H2AC, HIST1H2AG, HIST1H2AL, HIST1H2AM, HIST1H2BC, HIST1H2BD, HIST1H2BJ, HIST1H2BK, HIST1H2BO, HIST1H3A, HIST1H3B, HIST1H3C, HIST1H3D, HIST1H3E, HIST1H3F, HIST1H3G, HIST1H3H, HIST1H3I, HIST1H3J, HIST2H3C, HIST2H3D, HIST3H3, HLA-A, HLA-B, HNF1A, HOXB13, HRAS, HSD3B1, HSP90AA1, ICK, ICOSLG, ID3, IDH1, IDH2, IFNGR1, IGF1, IGF1R, IGF2, IKBKE, IKZF1, IKZF2, IKZF3, IL10, IL7R, INHA, INHBA, INPP4A, INPP4B, INPP5D (SHIP), INPPL1, INSR, IRF1, IRF2, IRF4, IRF8, IRS1, IRS2, JAK1, JAK2, JAK3, JARID2, JUN, K14, KAT6A (MYST 3), KAT6A (MYST3), KDM2B, KDM4C, KDM5A, KDM5C, KDM6A, KDR, KEAP1, KEL, KIF5B, KIT, KLF4, KLHL6, KMT2A, KMT2A (MLL), KMT2B, KMT2C, KMT20 (MLL3), KMT2D, KMT2D (MLL2), KNSTRN, KRAS, LAMP1, LATS1, LATS2, LEF1, LMO1, LRP1B, LRRK2, LTK, LYN, LZTR1, MAF, MAFB, MAGED1, MAGI2, MALT1, MAP2K1, MAP2K1 (MEK1), MAP2K2, MAP2K2 (MEK2), MAP2K4, MAP3, MAP3K1, MAP3K13, MAP3K14, MAP3K6, MAP3K7, MAPK1, MAPK3, MAPKAP1, MAX, MCL1, MDC1, MDM2, MDM4, MED12, MEF2B, MEF2C, MEK1, MEN1, MERTK, MET, MGA, MIB1, MITF, MKI67, MKNK1, MLH1, MLLT3, MPL, MRE11A, MRE11A, MSH2, MSH3, MSH6, MSI1, MSI2, MST1, MST1R, MTAP, MTOR, MUTYH, MYC, MYCL, MYCL (MYC L1), MYCL (MYCL1), MYCL1, MYCN, MYD88, MYO18A, MYOD1, NBN, NCOA3, NCOR1, NCOR2, NCSTN, NEGR1, NF1, NF2, NFE2L2, NFKB1A, NKX2-1, NKX3-1, NOD1, NOTCH1, NOTCH2, NOTCH3, NOTCH4, NPM1, NRAS, NRG1, NSD1, NT5C2, NTHL1, NTRK1, NTRK2, NTRK3, NUF2, NUP93, NUP98, P2RY8, PAG1, PAK1, PAK3, PAK7, PALB2, PARK2, PARP1, PARP2, PARP3, PASK, PAX3, PAX5, PAX7, PBRM1, PC, PCBP1, PCLO, PDCD1, PDCD1 (PD-1), PDCD11, PDCD1LG2, PDCD1LG2 (PD-L2), PDGFRA, PDGFRB, PDK1, PDPK1, PGR, PHF6, PHOX2B, PIK3C2B, PIK302G, PIK3C3, PIK3CA, PIK3CB, PIK3CD, PIK3CG, PIK3R1, PIK3R2, PIK3R3, PIM1, PLCG2, PLK2, PMAIP1, PMS1, PMS2, PNRC1, POLD1, POLE, POT1, PPARG, PPM1D, PPP2, PPP2R1A, PPP2R2A, PPP4R2, PPP6C, PRDM1, PRDM14, PREX2, PRKAR1A, PRKC1, PRKD1, PRKDC, PRSS8, PTCH1, PTEN, PTP4A1, PTPN11, PTPN2, PTPN6 (SHP-1), PTPRD, PTPRO, PTPRS, PTPRT, QK1, R1A, RAB35, RAC1, RAC2, RAD21, RAD50, RAD51, RAD51B, RAD51C, RAD51D, RAD52, RAD54L, RAF1, RANBP2, RARA, RASA1, RASGEF1A, RB1, RBM10, RECQL, RECQL4, REL, RELN, RET, RFWD2, RHEB, RHOA, RICTOR, RIT1, RNF43, ROS1, RPS6KA4, RPS6KB1, RPS6KB2, RPTOR, RRAGC, RRAS, RRAS2, RTEL1, RUNX1, RUNX1T1, RXRA, RYBP, SIPR2, SDHA, SDHAF2, SDHB, SDHC, SDHD, SERP2, SESN1, SESN2, SESN3, SETBP1, SETD2, SETD8, SF3B1, SGK1, SH2B3, SH2D1A, SHOC2, SHQ1, SLIT2, SLX4, SMAD2, SMAD3, SMAD4, SMARCA1, SMARCA4, SMARCB1, SMARCD1, SMC1A, SMC3, SMO, SMYD3, SNCA1P, SOCS1, SOCS2, SOCS3, SOS1, SOX10, SOX17, SOX2, SOX9, SPEN, SPOP, SPRED1, SPTA1, SRC, SRSF2, STAG2, STAT3, STAT4, STAT5A, STAT5B, STAT6, STK11, STK19, STK40, SUFU, SUZ12, SYK, TAF1, TAP1, TAP2, TBL1XR1, TBX3, TCEB1, TCF3, TCF3 (E2A), TCF7L2, TCL1A (TCL1), TEK, TERC, TERT, TERT Promoter, TET1, TET2, TFRC, TGFBR1, TGFBR2, TIPARP, TLL2, TMEM127, TMEM30A, TMPRSS2, TMSB4XP8 (TMSL3), TNFAIP3, TNFRSF11A, TNFRSF14, TNFRSF17, TOP1, TOP2A, TP53, TP53BP1, TP63, TRAF2, TRAF3, TRAF5, TRAF7, TSC1, TSC2, TSHR, TUSC3, TYK2, TYRO3, U2AF1, U2AF2, UPF1, VEGFA, VHL, VTCN1, WDR90, WHSC1, WHSC1 (MMSET or NSD2), WHSC1L1, WISP3, WT1, WWTR1, XBP1, XIAP, XPO1, XRCC2, YAP1, YES1, YY1AP1, ZBTB2, ZFHX3, ZMYM3, ZNF217, ZNF24 (ZSCAN3), ZNF703, ZRSR2, and any combination thereof.

In another embodiment, the genomic profile comprises one or more genes selected from the genes listed in Tables 2-17.

In one embodiment, TMB status based on genomic profiling is highly correlated with TMB status based on whole-exome or whole-genome sequencing. Evidence provided herein shows that the use of genomic profiling assays, such as the F1CDx assay, have concordance with whole-exome and/or whole genome sequencing assays. These data support the use of genomic profiling assays as a more efficient means of measuring TMB status, without forfeiting the prognostic qualities of TMB status.

TMB can be measured using a tissue biopsy sample or, alternatively, circulating tumor DNA (ctDNA), cfDNA (cell-free DNA), and/or a liquid biopsy sample. ctDNA can be used to measure TMB status according to whole-exome or whole-genome sequencing or genomic profiling using available methodologies, e.g., GRAIL, Inc.

A subject is identified as suitable for an immunotherapy, e.g., with an anti-PD-1 antibody or antigen-binding portion thereof or an anti-PD-L1 antibody or antigen-binding portion thereof, based on the measurement of TMB status and identification of a high TMB. In some embodiments, a TMB score is calculated as the total number of nonsynonymous missense mutations in a tumor, as measured by whole exome sequencing or whole genome sequencing. In one embodiment, the high TMB has a score of at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, or at least 500. In another embodiment, the high TMB has a score of at least 215, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, or at least 250. In a particular embodiment, the high TMB has a score of at least 243. In other embodiments, the high TMB has a score of at least 244. In some embodiments, the high TMB has a score of at least 245. In other embodiments, the high TMB has a score of at least 246. In other embodiments, the high TMB has a score of at least 247. In other embodiments, the high TMB has a score of at least 248. In other embodiments, the high TMB has a score of at least 249. In other embodiments, the high TMB has a score of at least 250. In other embodiments, the high TMB has a score of any integer between 200 and 300 or higher. In other embodiments, the high TMB has a score of any integer between 210 and 290 or higher. In other embodiments, the high TMB has a score of any integer between 220 and 280 or higher. In other embodiments, the high TMB has a score of any integer between 230 and 270 or higher. In other embodiments, the high TMB has a score of any integer between 235 and 265 or higher.

Alternatively, the high TMB can be a relative value rather than an absolute value. In some embodiments, the subject's TMB status is compared to a reference TMB value. In one embodiment, the subject's TMB status is within the highest fractile of the reference TMB value. In another embodiment, the subject's TMB status is within the top tertile of the reference TMB value.

In some embodiments, TMB status is expressed as the number of mutations per sample, per cell, per exome, or per length of DNA (e.g., Mb). In some embodiments, a tumor has a high TMB status if the tumor has at least about 50 mutations/tumor, at least about 55 mutations/tumor, at least about 60 mutations/tumor, at least about 65 mutations/tumor, at least about 70 mutations/tumor, at least about 75 mutations/tumor, at least about 80 mutations/tumor, at least about 85 mutations/tumor, at least about 90 mutations/tumor, at least about 95 mutations/tumor, at least about 100 mutations/tumor, at least about 105 mutations/tumor, at least about 110 mutations/tumor, at least about 115 mutations/tumor, or at least about 120 mutations/tumor. In some embodiments, a tumor has a high TMB status if the tumor has at least about 125 mutations/tumor, at least about 150 mutations/tumor, at least about 175 mutations/tumor, at least about 200 mutations/tumor, at least about 225 mutations/tumor, at least about 250 mutations/tumor, at least about 275 mutations/tumor, at least about 300 mutations/tumor, at least about 350 mutations/tumor, at least about 400 mutations/tumor, or at least about 500 mutations/tumor. In one particular embodiment, a tumor has a high TMB status if the tumor has at least about 100 mutations/tumor.

In some embodiments, a tumor has a high TMB status if the tumor has at least about 5 mutations per megabase of genes, e.g., genome sequenced according to a TMB assay, e.g., genome sequenced according to a FOUNDATIONONE® CDX™ assay, (mutations/Mb), at least about 6 mutations/Mb, at least about 7 mutations/Mb, at least about 8 mutations/Mb, at least about 9 mutations/Mb, at least about 10 mutations/Mb, at least about 11 mutations/Mb, at least about 12 mutations/Mb, at least about 13 mutations/Mb, at least about 14 mutations/Mb, at least about 15 mutations/Mb, at least about 20 mutations/Mb, at least about 25 mutations/Mb, at least about 30 mutations/Mb, at least about 35 mutations/Mb, at least about 40 mutations/Mb, at least about 45 mutations/Mb, at least about 50 mutations/Mb, at least about 75 mutations/Mb, or at least about 100 mutations/Mb. In certain embodiments, a tumor has a high TMB status if the tumor has at least about 5 mutations/Mb. In certain embodiments, a tumor has a high TMB status if the tumor has at least about 10 mutations/Mb. In some embodiments, a tumor has a high TMB status if the tumor has at least about 11 mutations/Mb. In some embodiments, a tumor has a high TMB status if the tumor has at least about 12 mutations/Mb. In some embodiments, a tumor has a high TMB status if the tumor has at least about 13 mutations/Mb. In some embodiments, a tumor has a high TMB status if the tumor has at least about 14 mutations/Mb. In certain embodiments, a tumor has a high TMB status if the tumor has at least about 15 mutations/Mb.

Because the number of mutations varies by tumor type and other ways (see Q4 and Q5), the values associated with “TMB high” and “TMB low” can differ across tumor types.

### PD-L1 Status

TMB status can be used alone or in combination with other factors as a means to predict a tumor's response to therapy and, in particular, treatment with an immuno-oncology agent, such as an anti-PD-1 antibody or an anti-PD-L1 antibody. In some embodiments, only the TMB status of a tumor is used to identify patients with a tumor more likely to respond to an immunotherapy, e.g., with an anti-PD-1 antibody or an anti-PD-L1 antibody. In other embodiments, the PD-L1 status and TMB status are used to identify patients with a tumor more likely to respond to an immunotherapy, e.g., with an anti-PD-1 antibody or an anti-PD-L1 antibody.

The PD-L1 status of a tumor in a subject can be measured prior to administering any composition or utilizing any method disclosed herein. PD-L1 expression can be determined by any methods known in the art.

In order to assess the PD-L1 expression, in one embodiment, a test tissue sample can be obtained from the patient who is in need of the therapy. In another embodiment, the assessment of PD-L1 expression can be achieved without obtaining a test tissue sample. In some embodiments, selecting a suitable patient includes (i) optionally providing a test tissue sample obtained from a patient with cancer of the tissue, the test tissue sample comprising tumor cells and/or tumor-infiltrating inflammatory cells; and (ii) assessing the proportion of cells in the test tissue sample that express PD-L1 on the surface of the cells based on an assessment that the proportion of cells in the test tissue sample that express PD-L1 on the cell surface is higher than a predetermined threshold level.

In any of the methods comprising the measurement of PD-L1 expression in a test tissue sample, however, it should be understood that the step comprising the provision of a test tissue sample obtained from a patient is an optional step. It should also be understood that in certain embodiments the “measuring” or “assessing” step to identify, or determine the number or proportion of, cells in the test tissue sample that express PD-L1 on the cell surface is performed by a transformative method of assaying for PD-L1 expression, for example by performing a reverse transcriptase-polymerase chain reaction (RT-PCR) assay or an IHC assay. In certain other embodiments, no transformative step is involved and PD-L1 expression is assessed by, for example, reviewing a report of test results from a laboratory. In certain embodiments, the steps of the methods up to, and including, assessing PD-L1 expression provides an intermediate result that can be provided to a physician or other healthcare provider for use in selecting a suitable candidate for the anti-PD-1 antibody or anti-PD-L1 antibody therapy. In certain embodiments, the steps that provide the intermediate result is performed by a medical practitioner or someone acting under the direction of a medical practitioner. In other embodiments, these steps are performed by an independent laboratory or by an independent person such as a laboratory technician.

In certain embodiments of any of the present methods, the proportion of cells that express PD-L1 is assessed by performing an assay to determine the presence of PD-L1 RNA. In further embodiments, the presence of PD-L1 RNA is determined by RT-PCR, in situ hybridization or RNase protection. In other embodiments, the proportion of cells that express PD-L1 is assessed by performing an assay to determine the presence of PD-L1 polypeptide. In further embodiments, the presence of PD-L1 polypeptide is determined by immunohistochemistry (IHC), enzyme-linked immunosorbent assay (ELISA), in vivo imaging, or flow cytometry. In some embodiments, PD-L1 expression is assayed by IHC. In other embodiments of all of these methods, cell surface expression of PD-L1 is assayed using, e.g., IHC or in vivo imaging.

Imaging techniques have provided important tools in cancer research and treatment. Recent developments in molecular imaging systems, including positron emission tomography (PET), single-photon emission computed tomography (SPECT), fluorescence reflectance imaging (FRI), fluorescence-mediated tomography (FMT), bioluminescence imaging (BLI), laser-scanning confocal microscopy (LSCM) and multiphoton microscopy (MPM), will likely herald even greater use of these techniques in cancer research. Some of these molecular imaging systems allow clinicians to not only see where a tumor is located in the body, but also to visualize the expression and activity of specific molecules, cells, and biological processes that influence tumor behavior and/or responsiveness to therapeutic drugs (Condeelis and Weissleder, “In vivo imaging in cancer,” Cold Spring Harb. Perspect. Biol. 2 (12): a003848 (2010)). Antibody specificity, coupled with the sensitivity and resolution of PET, makes immunoPET imaging particularly attractive for monitoring and assaying expression of antigens in tissue samples (McCabe and Wu, “Positive progress in immunoPET—not just a coincidence,” Cancer Biother. Radiopharm. 25 (3): 253-61 (2010); Olafsen et al., “ImmunoPET imaging of B-cell lymphoma using 124I-anti-CD20 scFv dimers (diabodies),” Protein Eng. Des. Sel. 23 (4): 243-9 (2010)). In certain embodiments of any of the present methods, PD-L1 expression is assayed by immunoPET imaging. In certain embodiments of any of the present methods, the proportion of cells in a test tissue sample that express PD-L1 is assessed by performing an assay to determine the presence of PD-L1 polypeptide on the surface of cells in the test tissue sample. In certain embodiments, the test tissue sample is a FFPE tissue sample. In other embodiments, the presence of PD-L1 polypeptide is determined by IHC assay. In further embodiments, the IHC assay is performed using an automated process. In some embodiments, the IHC assay is performed using an anti-PD-L1 monoclonal antibody to bind to the PD-L1 polypeptide. In certain embodiments, the anti-PD-L1 monoclonal antibody is selected from the group consisting of 28-8, 28-1, 28-12, 29-8, 5H1, and any combination thereof. See WO/2013/173223, which is incorporated by reference herein in its entirety.

In one embodiment of the present methods, an automated IHC method is used to assay the expression of PD-L1 on the surface of cells in FFPE tissue specimens. The presence of human PD-L1 antigen can be measured in a test tissue sample by contacting the test sample, and a negative control sample (e.g., normal tissue), with a monoclonal antibody that specifically binds to human PD-L1, under conditions that allow for formation of a complex between the antibody or portion thereof and human PD-L1. In certain embodiments, the test and control tissue samples are FFPE samples. The formation of a complex is then detected, wherein a difference in complex formation between the test sample and the negative control sample is indicative of the presence of human PD-L1 antigen in the sample. Various methods are used to quantify PD-L1 expression.

In a particular embodiment, the automated IHC method comprises: (a) deparaffinizing and rehydrating mounted tissue sections in an autostainer; (b) retrieving antigen using a decloaking chamber and pH 6 buffer, heated to 110° C. for 10 min; (c) setting up reagents on an autostainer; and (d) running the autostainer to include steps of neutralizing endogenous peroxidase in the tissue specimen; blocking non-specific protein-binding sites on the slides; incubating the slides with primary antibody; incubating with a post primary blocking agent; incubating with NovoLink Polymer; adding a chromogen substrate and developing; and counterstaining with hematoxylin.

For assessing PD-L1 expression in tumor tissue samples, a pathologist examines the number of membrane PD-L1⁺ tumor cells in each field under a microscope and mentally estimates the percentage of cells that are positive, then averages them to come to the final percentage. The different staining intensities are defined as 0/negative, 1+/weak, 2+/moderate, and 3+/strong. Typically, percentage values are first assigned to the 0 and 3+ buckets, and then the intermediate 1+ and 2+ intensities are considered. For highly heterogeneous tissues, the specimen is divided into zones, and each zone is scored separately and then combined into a single set of percentage values. The percentages of negative and positive cells for the different staining intensities are determined from each area and a median value is given to each zone. A final percentage value is given to the tissue for each staining intensity category: negative, 1+, 2+, and 3+. The sum of all staining intensities needs to be 100%. In one embodiment, the threshold number of cells that needs to be PD-L1 positive is at least about 100, at least about 125, at least about 150, at least about 175, or at least about 200 cells. In certain embodiments, the threshold number or cells that needs to be PD-L1 positive is at least about 100 cells.

Staining is also assessed in tumor-infiltrating inflammatory cells such as macrophages and lymphocytes. In most cases macrophages serve as an internal positive control since staining is observed in a large proportion of macrophages. While not required to stain with 3+ intensity, an absence of staining of macrophages should be taken into account to rule out any technical failure. Macrophages and lymphocytes are assessed for plasma membrane staining and only recorded for all samples as being positive or negative for each cell category. Staining is also characterized according to an outside/inside tumor immune cell designation. “Inside” means the immune cell is within the tumor tissue and/or on the boundaries of the tumor region without being physically intercalated among the tumor cells. “Outside” means that there is no physical association with the tumor, the immune cells being found in the periphery associated with connective or any associated adjacent tissue.

In certain embodiments of these scoring methods, the samples are scored by two pathologists operating independently, and the scores are subsequently consolidated. In certain other embodiments, the identification of positive and negative cells is scored using appropriate software.

A histoscore is used as a more quantitative measure of the IHC data. The histoscore is calculated as follows:

Histoscore=[(% tumor×1 (low intensity))+(% tumor×2 (medium intensity))+(% tumor×3 (high intensity)]

To determine the histoscore, the pathologist estimates the percentage of stained cells in each intensity category within a specimen. Because expression of most biomarkers is heterogeneous the histoscore is a truer representation of the overall expression. The final histoscore range is 0 (no expression) to 300 (maximum expression).

An alternative means of quantifying PD-L1 expression in a test tissue sample IHC is to determine the adjusted inflammation score (AIS) score defined as the density of inflammation multiplied by the percent PD-L1 expression by tumor-infiltrating inflammatory cells (Taube et al., “Colocalization of inflammatory response with B7-h1 expression in human melanocytic lesions supports an adaptive resistance mechanism of immune escape,” Sci. Transl. Med. 4 (127): 127ra37 (2012)).

In one embodiment, the PD-L1 expression level of a tumor is at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 75%, at least about 80%, at least about 85%, at least about 90%, at least about 95%, or about 100%. In another embodiment, the PD-L1 status of a tumor is at least about 1%. In other embodiments, the PD-L1 status of the subject is at least about 5%. In a certain embodiment, the PD-L1 status of a tumor is at least about 10%. In one embodiment, the PD-L1 status of the tumor is at least about 25%. In a particular embodiment, the PD-L1 status of the tumor is at least about 50%.

“PD-L1 positive” as used herein can be interchangeably used with “PD-L1 expression of at least about 1%”. In one embodiment, the PD-L1 positive tumors can thus have at least about 1%, at least about 2%, at least about 5%, at least about 10%, at least about 20%, at least about 25%, at least about 30%, at least about 40%, at least about 50%, at least about 60%, at least about 70%, at least about 75%, at least about 80%, at least about 85%, at least about 90%, at least about 95%, or about 100% of the tumor cells expressing PD-L1 as measured by an automated IHC. In certain embodiments, “PD-L1 positive” means that there are at least 100 cells that express PD-L1 on the surface of the cells.

In one embodiment, a PD-L1 positive tumor with high TMB has a greater likelihood of response to therapy with an anti-PD-1 antibody than a tumor with only high TMB, only PD-L1 positive expression, or neither. In one embodiment, the tumor has at least about 1%, about 5%, about 10%, about 15%, about 20%, about 25%, about 30%, about 35%, about 40%, about 45%, or about 50% PD-L1 expression. In a particular embodiment, a tumor with ≥50% PD-L1 expression and a high TMB status is more likely to respond to therapy with an anti-PD-1 antibody than a tumor with only high TMB, only ≥50% PD-L1 expression, or neither.

In certain embodiments, the tumor in the subject suitable for the immunotherapy, e.g., an anti-PD-1 antibody treatment, in this disclosure does not express PD-L1 (less than 1%, less than 2%, less than 3%, less than 4%, or less than 5% membranous PD-L1). In some embodiments, the methods of the present disclosure are irrelevant to the PD-L1 expression.

### MSI Status

TMB status can be used alone or in combination with other factors, e.g., MSI status, as a means to predict a tumor's response to therapy and, in particular, treatment with an immuno-oncology agent, such as an anti-PD-1 antibody or an anti-PD-L1 antibody. In one embodiment, the MSI status is part of the TMB status. In other embodiments, the MSI status is measured separately from the TMB status.

Microsatellite instability is the condition of genetic hypermutability that results from impaired DNA mismatch repair (MMR). The presence of MSI represents phenotypic evidence that MMR is not functioning normally. In most cases, the genetic basis for instability in MSI tumors is an inherited germline alteration in any one of the five human MMR genes: MSH2, MLH1, MSH6, PMS2, and PMS1. In certain embodiments, the subject receiving tumor (e.g., colon tumor) treatment has a high degree of microsatellite instability (MSI-H) and has at least one mutation in genes MSH2, MLH1, MSH6, PMS2, or PMS1. In other embodiments, subjects receiving tumor treatment within a control group have no microsatellite instability (MSS or MSI stable) and has no mutation in genes MSH2, MLH1, MSH6, PMS2, and PMS1.

In one embodiment, the subject suitable for the immunotherapy has a high TMB status and a MSI-H tumor. As used herein, MSI-H tumors mean tumors having greater than at least about 30% of unstable MSI biomarkers. In some embodiments, the tumor is derived from a colorectal cancer. In some embodiments, the tumor is a colorectal cancer with MSI-H when a germline alteration is detected in at least two, at least three, at least four, or at least five MMR genes. In other embodiments, the tumor is a colorectal cancer with MSI-H when a germline alteration is detected in at least 30% of five or more MMR genes. In some embodiments, a germline alternation in MMR genes is measured by a polymerase chain reaction. In other embodiments, the tumor is a colorectal cancer with MSI-H when at least one protein encoded by DNA MMR genes is not detected in the tumor. In some embodiments, the at least one protein encoded by DNA MMR genes is detected by an immunohistochemistry.

### Treatment Methods of the Disclosure

The present disclosure is directed to a method for treating a subject afflicted with a tumor having a high tumor mutation burden (TMB) status comprising administering to the subject an immunotherapy. In some embodiments, the immunotherapy comprises administering to the subject an antibody or an antigen-binding portion thereof. In some embodiments, the method comprises treating a subject afflicted with a tumor having a high TMB status comprising administering to the subject an antibody or an antigen binding fragment thereof that specifically binds a protein selected from the group consisting of PD-1, PD-L1, CTLA-4, LAG3, TIGIT, TIM3, NKG2a, OX40, ICOS, MICA, CD137, KIR, TGFβ, IL-10, IL-8, B7-H4, Fas ligand, CXCR4, mesothelin, CD27, GITR, and any combination thereof. In certain embodiments, the method comprises treating a subject afflicted with a tumor having a high TMB status comprising administering to the subject an antibody or an antigen binding fragment thereof that specifically binds PD-1 or PD-L1.

Certain cancer types have a higher frequency of mutations and, thus, have a high TMB. (Alexandrov et al., Nature (2013) 500:415-421.) Non-limiting examples of cancers with a high TMB include melanoma, lung, bladder, and gastrointestinal cancers. In some embodiments, the tumor is lung cancer. In one embodiment, the lung cancer is non-small cell lung cancer (NSCLC). In one embodiment, the NSCLC has a squamous histology. In another embodiment, the NSCLC has a non-squamous histology. In other embodiments, the tumor is selected from renal cell carcinoma, ovarian cancer, colorectal cancer, gastrointestinal cancer, esophageal cancer, bladder cancer, lung cancer, and melanoma. It should be understood that the methods disclosed herein encompass solid tumors as well as blood cancers.

The methods of treatment disclosed herein can provide an improved clinical response and/or clinical benefit for subjects afflicted with a tumor and, in particular, subjects having a tumor with a high TMB. High TMB can be related to neoantigen burden, i.e., the number of neoantigens and T-cell reactivity and, thus, an immune-mediated anti-tumor response. Accordingly, high TMB is a factor that can be used, alone or in combination with other factors, to identity tumors (and patients having such tumors) more likely to benefit from therapy with an anti-PD-1 antibody and/or an anti-PD-L1 antibody, e.g., as compared to current standard of care therapies.

In one embodiment, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the administration. In another embodiment, the subject exhibits an overall survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the administration. In yet another embodiment, the subject exhibits an objective response rate of at least about 30%, about 35%, about 40%, about 45%, about 50%, about 55%, about 60%, about 65%, about 70%, about 75%, about 80%, about 85%, about 90%, about 95%, or about 100%.

### Anti-PD-1/Anti-PD-L1 Treatment

Certain aspects of the present disclosure are directed to a method for treating a subject afflicted with a tumor having a high tumor mutation burden (TMB) status comprising administering to the subject an immunotherapy, wherein the immunotherapy comprises an anti-PD-1 antibody or an anti-PD-L1 antibody. The method can further comprise measuring the TMB status of a biological sample obtained from the subject. Additionally, the disclosure contemplates administering an anti-PD-1 antibody or an anti-PD-L1 antibody to a subject identified as suitable for such therapy, e.g., based on measurement of a high TMB.

In one embodiment, the anti-PD-1 antibody cross-competes with nivolumab for binding to human PD-1. In another embodiment, the anti-PD-1 antibody binds to the same epitope as nivolumab. In a particular embodiment, the anti-PD-1 antibody is nivolumab. In another particular embodiment, the anti-PD-1 antibody is pembrolizumab. Additional anti-PD-1 antibodies are described elsewhere herein. In other embodiments, anti-PD-1 antibodies useful for the disclosure are disclosed elsewhere herein. In some embodiments, an anti-PD-L1 antibody can replace an anti-PD-1 antibody. Exemplary anti-PD-L1 antibodies useful for the methods of the disclosure are described elsewhere herein.

In some embodiments, the anti-PD-1 antibody or an anti-PD-L1 antibody is a chimeric antibody, a humanized antibody, a human antibody, or an antigen-binding portion thereof. In other embodiments, the anti-PD-1 antibody or an anti-PD-L1 antibody comprises a heavy chain constant region of a human IgG1 isotype or a human IgG4 isotype.

### Anti-PD-1 Antibodies Useful for the Disclosure

Anti-PD-1 antibodies that are known in the art can be used in the presently described compositions and methods. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-γ production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some embodiments, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

In some embodiments, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; see Si-Yang Liu et al., J. Hematol. Oncol. 10:136 (2017)), BGB-A317 (Beigene; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), and IBI308 (Innovent; see WO 2017/024465, WO 2017/025016, WO 2017/132825, and WO 2017/133540).

In one embodiment, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2 (9): 846-56).

In certain embodiments, the anti-PD-1 antibody comprises a heavy chain variable region comprising an amino acid sequence at least 80%, at least 85%, at least 90%, at least 95%, at least 95%, at least 96%, at least 97%, at least 98%, at least 99%, or 100% identical to amino acids having the sequence set forth in SEQ ID NO: 11 (and/or having three CDRs comprising amino acids 31 to 35 of SEQ ID NO: 11, amino acids 55 to 66 of SEQ ID NO: 11, and amino acids 99 to 102 of SEQ ID NO: 11) and a light chain variable region comprising amino acids having the sequence set forth in an amino acid sequence at least 80%, at least 85%, at least 90%, at least 95%, at least 95%, at least 96%, at least 97%, at least 98%, at least 99%, or 100% identical to SEQ ID NO: 12 (and/or having three CDRs comprising amino acids 24 to 34 of SEQ ID NO: 12, amino acids 50 to 56 of SEQ ID NO: 12, and amino acids 89 to 97 of SEQ ID NO: 12).

Heavy Chain:
(SEQ ID NO: 11)
QVQLVESGGGVVQPGRSLRLDCKASGITFSNSGMHWVRQA
PGKGLEWVAVIWYDGSKRYYADSVKGRFTISRDNSKNTLFL
QMNSLRAEDTAVYYCATNDDYWGQGTLVTVSS.
CDRs underlined.
Light Chain:
(SEQ ID NO: 12)
EIVLTQSPATLSLSPGERATLSCRASQSVSSYLAWYQQKPGQ
APRLLIYDASNRATGIPARFSGSGSGTDFTLTISSLEPEDFA
VYYCQQSSNWPRTFGQGTKVEIK.
CDRs underlined.

In another embodiment, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

Anti-PD-1 antibodies usable in the disclosed compositions and methods also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some embodiments, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain embodiments, the antibodies that cross-compete for binding to human PD-1 with, or bind to the same epitope region of human PD-1 antibody, nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies usable in the compositions and methods of the disclosure also include antigen-binding portions of the above antibodies. It has been amply demonstrated that the antigen-binding function of an antibody can be performed by fragments of a full-length antibody.

Anti-PD-1 antibodies suitable for use in the disclosed compositions and methods are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain embodiments, the anti-PD-1 antibody or antigen-binding portion thereof cross-competes with nivolumab for binding to human PD-1.

In some embodiments, the anti-PD-1 antibody is administered at a dose ranging from 0.1 mg/kg to 20.0 mg/kg body weight once every 2, 3, 4, 5, 6, 7, or 8 weeks, e.g., 0.1 mg/kg to 10.0 mg/kg body weight once every 2, 3, or 4 weeks. In other embodiments, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 2 weeks. In other embodiments, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 3 weeks. In one embodiment, the anti-PD-1 antibody is administered at a dose of about 5 mg/kg body weight about once every 3 weeks. In another embodiment, the anti-PD-1 antibody, e.g., nivolumab, is administered at a dose of about 3 mg/kg body weight about once every 2 weeks. In other embodiments, the anti-PD-1 antibody, e.g., pembrolizumab, is administered at a dose of about 2 mg/kg body weight about once every 3 weeks.

The anti-PD-1 antibody useful for the present disclosure can be administered as a flat dose. In one embodiment, the anti-PD-1 antibody is administered as a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, or at least about 550 mg at a dosing interval of about 1, 2, 3, 4, 5, 6, 7, 8, 9, or 10 weeks. In another embodiments, the anti-PD-1 antibody is administered as a flat dose of about 200 mg to about 800 mg, about 200 mg to about 700 mg, about 200 mg to about 600 mg, about 200 mg to about 500 mg, at a dosing interval of about 1, 2, 3, or 4 weeks.

In some embodiments, the anti-PD-1 antibody is administered as a flat dose of about 200 mg at about once every 3 weeks. In other embodiments, the anti-PD-1 antibody is administered as a flat dose of about 240 mg at about once every 2 weeks. In certain embodiments, the anti-PD-1 antibody is administered as a flat dose of about 480 mg at about once every 4 weeks.

### Anti-PD-L1 Antibodies Useful for the Disclosure

Anti-PD-L1 antibodies that are known in the art can be used in the compositions and methods of the present disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some embodiments, at least five, of the preceding characteristics.

In certain embodiments, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31 (suppl): 3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), and CK-301 (Checkpoint Therapeutics; see Gorelik et al., AACR: Abstract 4606 (April 2016)).

In certain embodiments, the anti-PD-L1 antibody is atezolizumab (TECENTRIQ®). Atezolizumab is a fully humanized IgG1 monoclonal anti-PD-L1 antibody.

In certain embodiments, the anti-PD-L1 antibody is durvalumab (IMFINZI™). Durvalumab is a human IgG1 kappa monoclonal anti-PD-L1 antibody.

In certain embodiments, the anti-PD-L1 antibody is avelumab (BAVENCIO®). Avelumab is a human IgG1 lambda monoclonal anti-PD-L1 antibody.

Anti-PD-L1 antibodies usable in the disclosed compositions and methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some embodiments, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain embodiments, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the compositions and methods of the disclosure also include antigen-binding portions of the above antibodies. It has been amply demonstrated that the antigen-binding function of an antibody can be performed by fragments of a full-length antibody.

Anti-PD-L1 antibodies suitable for use in the disclosed compositions and methods are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain embodiments, the anti-PD-L1 antibody or antigen-binding portion thereof cross-competes with atezolizumab, durvalumab, and/or avelumab for binding to human PD-L1.

The anti-PD-L1 antibody useful for the present disclosure can be any anti-PD-L1 antibody that specifically binds to PD-L1, e.g., antibodies that cross-compete with durvalumab, avelumab, or atezolizumab for binding to human PD-1, e.g., an antibody that binds to the same epitope as durvalumab, avelumab, or atezolizumab. In a particular embodiment, the anti-PD-L1 antibody is durvalumab. In other embodiments, the anti-PD-L1 antibody is avelumab. In some embodiments, the anti-PD-L1 antibody is atezolizumab.

In some embodiments, the anti-PD-L1 antibody is administered at a dose ranging from about 0.1 mg/kg to about 20.0 mg/kg body weight, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, about once every 2, 3, 4, 5, 6, 7, or 8 weeks.

In some embodiments, the anti-PD-L1 antibody is administered at a dose of about 15 mg/kg body weight at about once every 3 weeks. In other embodiments, the anti-PD-L1 antibody is administered at a dose of about 10 mg/kg body weight at about once every 2 weeks.

In other embodiments, the anti-PD-L1 antibody useful for the present disclosure is a flat dose. In some embodiments, the anti-PD-L1 antibody is administered as a flat dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, or at least about 1400 mg, at a dosing interval of about 1, 2, 3, or 4 weeks. In some embodiments, the anti-PD-L1 antibody is administered as a flat dose of about 1200 mg at about once every 3 weeks. In other embodiments, the anti-PD-L1 antibody is administered as a flat dose of about 800 mg at about once every 2 weeks.

### Anti-CTLA-4 Antibodies

Certain aspects of the present disclosure are directed to a method for treating a subject afflicted with a tumor having a high tumor mutation burden (TMB) status comprising administering to the subject immunotherapy, wherein the immunotherapy comprises an anti-CTLA-4 antibody. The method can further comprise measuring the TMB status of a biological sample obtained from the subject. Additionally, the disclosure contemplates administering an anti-CTLA-4 antibody to a subject identified as suitable for such therapy, e.g., based on measurement of a high TMB.

Anti-CTLA-4 antibodies that are known in the art can be used in the compositions and methods of the present disclosure. Anti-CTLA-4 antibodies of the instant disclosure bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Human monoclonal antibodies that bind specifically to CTLA-4 with high affinity have been disclosed in U.S. Pat. No. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. 6,984,720 have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 10¹¹ M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (kₐ) of at least about 10³, about 10⁴, or about 10⁵ ml s⁻¹; and (d) inhibits the binding of CTLA-4 to B7-1 (CD80) and B7-2 (CD86). Anti-CTLA-4 antibodies useful for the present disclosure include monoclonal antibodies that bind specifically to human CTLA-4 and exhibit at least one, at least two, or at least three of the preceding characteristics.

In certain embodiments, the anti-CTLA-4 antibody is selected from the group consisting of ipilimumab (also known as YERVOY®, MDX-010, 10D1; see U.S. Pat. No. 6,984,720), MK-1308 (Merck), AGEN-1884 (Agenus Inc.; see WO 2016/196237), and tremelimumab (AstraZeneca; also known as ticilimumab, CP-675,206; see WO 2000/037504 and Ribas, Update Cancer Ther. 2 (3): 133-39 (2007)). In particular embodiments, the anti-CTLA-4 antibody is ipilimumab.

In particular embodiments, the anti-CTLA-4 antibody is ipilimumab for use in the compositions and methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma.

In particular embodiments, the anti-CTLA-4 antibody is tremelimumab.

In particular embodiments, the anti-CTLA-4 antibody is MK-1308.

In particular embodiments, the anti-CTLA-4 antibody is AGEN-1884.

Anti-CTLA-4 antibodies usable in the disclosed compositions and methods also include isolated antibodies that bind specifically to human CTLA-4 and cross-compete for binding to human CTLA-4 with any anti-CTLA-4 antibody disclosed herein, e.g., ipilimumab and/or tremelimumab. In some embodiments, the anti-CTLA-4 antibody binds the same epitope as any of the anti-CTLA-4 antibodies described herein, e.g., ipilimumab and/or tremelimumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., ipilimumab and/or tremelimumab, by virtue of their binding to the same epitope region of CTLA-4. Cross-competing antibodies can be readily identified based on their ability to cross-compete with ipilimumab and/or tremelimumab in standard CTLA-4 binding assays such as Biacore analysis, ELISA assays or flow cytometry (see, e.g., WO 2013/173223).

In certain embodiments, the antibodies that cross-compete for binding to human CTLA-4 with, or bind to the same epitope region of human CTLA-4 antibody as, ipilimumab and/or tremelimumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-CTLA-4 antibodies usable in the compositions and methods of the disclosure also include antigen-binding portions of the above antibodies. It has been amply demonstrated that the antigen-binding function of an antibody can be performed by fragments of a full-length antibody.

Anti-CTLA-4 antibodies suitable for use in the disclosed methods or compositions are antibodies that bind to CTLA-4 with high specificity and affinity, block the activity of CTLA-4, and disrupt the interaction of CTLA-4 with a human B7 receptor. In any of the compositions or methods disclosed herein, an anti-CTLA-4 “antibody” includes an antigen-binding portion or fragment that binds to CTLA-4 and exhibits the functional properties similar to those of whole antibodies in inhibiting the interaction of CTLA-4 with a human B7 receptor and up-regulating the immune system. In certain embodiments, the anti-CTLA-4 antibody or antigen-binding portion thereof cross-competes with ipilimumab and/or tremelimumab for binding to human CTLA-4.

In some embodiments, the anti-CTLA-4 antibody or antigen-binding portion thereof is administered at a dose ranging from 0.1 mg/kg to 10.0 mg/kg body weight once every 2, 3, 4, 5, 6, 7, or 8 weeks. In some embodiments, the anti-CTLA-4 antibody or antigen-binding portion thereof is administered at a dose of 1 mg/kg or 3 mg/kg body weight once every 3, 4, 5, or 6 weeks. In one embodiment, the anti-CTLA-4 antibody or antigen-binding portion thereof is administered at a dose of 3 mg/kg body weight once every 2 weeks. In another embodiment, the anti-PD-1 antibody or antigen-binding portion thereof is administered at a dose of 1 mg/kg body weight once every 6 weeks.

In some embodiments, the anti-CTLA-4 antibody or antigen-binding portion thereof is administered as a flat dose. In one embodiment, the anti-CTLA-4 antibody or antigen-binding portion thereof is administered as a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, or at least about 550 mg. In another embodiment, the anti-CTLA-4 antibody or antigen-binding portion thereof is administered as a flat dose about once every 1, 2, 3, 4, 5, 6, 7, or 8 weeks.

### Anti-LAG-3 Antibodies

Certain aspects of the present disclosure are directed to a method for treating a subject afflicted with a tumor having a high TMB status comprising administering to the subject immunotherapy, wherein the immunotherapy comprises an anti-LAG-3 antibody or antigen-binding portion thereof. The method can further comprise measuring the TMB status of a biological sample obtained from the subject. Additionally, the disclosure contemplates administering an anti-LAG-3 antibody or antigen-binding portion thereof to a subject identified as suitable for such therapy, e.g., based on measurement of a high TMB.

Anti-LAG-3 antibodies of the instant disclosure bind to human LAG-3. Antibodies that bind to LAG-3 have been disclosed in Int'l Publ. No. WO/2015/042246 and U.S. Publ. Nos. 2014/0093511 and 2011/0150892. An exemplary LAG-3 antibody useful in the present disclosure is 25F7 (described in U.S. Publ. No. 2011/0150892). An additional exemplary LAG-3 antibody useful in the present disclosure is BMS-986016. In one embodiment, an anti-LAG-3 antibody useful for the composition cross-competes with 25F7 or BMS-986016. In another embodiment, an anti-LAG-3 antibody useful for the composition binds to the same epitope as 25F7 or BMS-986016. In other embodiments, an anti-LAG-3 antibody comprises six CDRs of 25F7 or BMS-986016.

### Anti-CD137 Antibodies

Certain aspects of the present disclosure are directed to a method for treating a subject afflicted with a tumor having a high TMB status comprising administering to the subject immunotherapy, wherein the immunotherapy comprises an anti-CD137 antibody or antigen-binding portion thereof. The method can further comprise measuring the TMB status of a biological sample obtained from the subject. Additionally, the disclosure contemplates administering an anti-CD137 antibody or antigen-binding portion thereof to a subject identified as suitable for such therapy, e.g., based on measurement of a high TMB.

Anti-CD137 antibodies specifically bind to and activate CD137-expressing immune cells, stimulating an immune response, in particular a cytotoxic T cell response, against tumor cells. Antibodies that bind to CD137 have been disclosed in U.S. Publ. No. 2005/0095244 and U.S. Pat. Nos. 7,288,638, 6,887,673, 7,214,493, 6,303,121, 6,569,997, 6,905,685, 6,355,476, 6,362,325, 6,974,863, and 6,210,669.

In some embodiments, the anti-CD137 antibody is urelumab (BMS-663513), described in U.S. Pat. No. 7,288,638 (20H4.9-IgG4 [10C7 or BMS-663513]). In some embodiments, the anti-CD137 antibody is BMS-663031 (20H4.9-IgG1), described in U.S. Pat. No. 7,288,638. In some embodiments, the anti-CD137 antibody is 4E9 or BMS-554271, described in U.S. Pat. No. 6,887,673. In some embodiments, the anti-CD137 antibody is an antibody disclosed in U.S. Pat. Nos. 7,214,493; 6,303,121; 6,569,997; 6,905,685; or 6,355,476. In some embodiments, the anti-CD137 antibody is 1D8 or BMS-469492; 3H3 or BMS-469497; or 3E1, described in U.S. Pat. No. 6,362,325. In some embodiments, the anti-CD137 antibody is an antibody disclosed in issued U.S. Pat. No. 6,974,863 (such as 53A2). In some embodiments, the anti-CD137 antibody is an antibody disclosed in issued U.S. Pat. No. 6,210,669 (such as 1D8, 3B8, or 3E1). In some embodiments, the antibody is Pfizer's PF-05082566 (PF-2566). In other embodiments, an anti-CD137 antibody useful for the disclosure cross-competes with the anti-CD137 antibodies disclosed herein. In some embodiments, an anti-CD137 antibody binds to the same epitope as the anti-CD137 antibody disclosed herein. In other embodiments, an anti-CD137 antibody useful in the disclosure comprises six CDRs of the anti-CD137 antibodies disclosed herein.

### Anti-KIR Antibodies

Certain aspects of the present disclosure are directed to a method for treating a subject afflicted with a tumor having a high TMB status comprising administering to the subject immunotherapy, wherein the immunotherapy comprises an anti-KIR antibody or antigen-binding portion thereof. The method can further comprise measuring the TMB status of a biological sample obtained from the subject. Additionally, the disclosure contemplates administering an anti-KIR antibody or antigen-binding portion thereof to a subject identified as suitable for such therapy, e.g., based on measurement of a high TMB.

Antibodies that bind specifically to KIR block the interaction between Killer-cell immunoglobulin-like receptors (KIR) on NK cells with their ligands. Blocking these receptors facilitates activation of NK cells and, potentially, destruction of tumor cells by the latter. Examples of anti-KIR antibodies have been disclosed in Int'l Publ. Nos. WO/2014/055648, WO 2005/003168, WO 2005/009465, WO 2006/072625, WO 2006/072626, WO 2007/042573, WO 2008/084106, WO 2010/065939, WO 2012/071411 and WO/2012/160448.

One anti-KIR antibody useful in the present disclosure is lirilumab (also referred to as BMS-986015, IPH2102, or the S241P variant of 1-7F9), first described in Int'l Publ. No. WO 2008/084106. An additional anti-KIR antibody useful in the present disclosure is 1-7F9 (also referred to as IPH2101), described in Int'l Publ. No. WO 2006/003179. In one embodiment, an anti-KIR antibody for the present composition cross competes for binding to KIR with lirilumab or I-7F9. In another embodiment, an anti-KIR antibody binds to the same epitope as lirilumab or I-7F9. In other embodiments, an anti-KIR antibody comprises six CDRs of lirilumab or I-7F9.

### Anti-GITR Antibodies

Certain aspects of the present disclosure are directed to a method for treating a subject afflicted with a tumor having a high TMB status comprising administering to the subject immunotherapy, wherein the immunotherapy comprises an anti-GITR antibody or antigen-binding portion thereof. The method can further comprise measuring the TMB status of a biological sample obtained from the subject. Additionally, the disclosure contemplates administering an anti-GITR antibody or antigen-binding portion thereof to a subject identified as suitable for such therapy, e.g., based on measurement of a high TMB.

Anti-GITR antibodies can be any anti-GITR antibody that binds specifically to human GITR target and activates the glucocorticoid-induced tumor necrosis factor receptor (GITR). GITR is a member of the TNF receptor superfamily that is expressed on the surface of multiple types of immune cells, including regulatory T cells, effector T cells, B cells, natural killer (NK) cells, and activated dendritic cells (“anti-GITR agonist antibodies”). Specifically, GITR activation increases the proliferation and function of effector T cells, as well as abrogating the suppression induced by activated T regulatory cells. In addition, GITR stimulation promotes anti-tumor immunity by increasing the activity of other immune cells such as NK cells, antigen presenting cells, and B cells. Examples of anti-GITR antibodies have been disclosed in Int'l Publ. Nos. WO/2015/031667, WO2015/184,099, WO2015/026,684, WO11/028683 and WO/2006/105021, U.S. Pat. Nos. 7,812,135 and 8,388,967 and U.S. Publ. Nos. 2009/0136494, 2014/0220002, 2013/0183321 and 2014/0348841.

In one embodiment, an anti-GITR antibody useful in the present disclosure is TRX518 (described in, for example, Schaer et al. Curr Opin Immunol. (2012) April; 24 (2): 217-224, and WO/2006/105021). In another embodiment, the anti-GITR antibody is selected from MK4166, MK1248, and antibodies described in WO11/028683 and U.S. Pat. No. 8,709,424, and comprising, e.g., a VH chain comprising SEQ ID NO: 104 and a VL chain comprising SEQ ID NO: 105 (wherein the SEQ ID NOs are from WO11/028683 or U.S. Pat. No. 8,709,424). In certain embodiments, an anti-GITR antibody is an anti-GITR antibody that is disclosed in WO2015/031667, e.g., an antibody comprising VH CDRs 1-3 comprising SEQ ID NOs: 31, 71 and 63 of WO2015/031667, respectively, and VL CDRs 1-3 comprising SEQ ID NOs: 5, 14 and 30 of WO2015/031667. In certain embodiments, an anti-GITR antibody is an anti-GITR antibody that is disclosed in WO2015/184099, e.g., antibody Hum231 #1 or Hum231 #2, or the CDRs thereof, or a derivative thereof (e.g., pab 1967, pab 1975 or pab 1979). In certain embodiments, an anti-GITR antibody is an anti-GITR antibody that is disclosed in JP2008278814, WO09/009116, WO2013/039954, US20140072566, US20140072565, US20140065152, or WO2015/026684, or is INBRX-110 (INHIBRx), LKZ-145 (Novartis), or MEDI-1873 (MedImmune). In certain embodiments, an anti-GITR antibody is an anti-GITR antibody that is described in PCT/US2015/033991 (e.g., an antibody comprising the variable regions of 28F3, 18E10 or 19D3). For example, an anti-GITR antibody may be an antibody comprising the following VH and VL chains or the CDRs thereof:

VH:
(SEQ ID NO: 1)
QVQLVESGGGVVQPGRSLRLSCAASGFTFSSYGMHWVRQAPG
KGLEWVAVIWYEGSNKYYADSVKGRFTISRDNSKNTLYLQMN
SLRAEDTAVYYCARGGSMVRGDYYYGMDVWGQGTTVTVS,
and
VL:
(SEQ ID NO: 2)
AIQLTQSPSSLSASVGDRVTITCRASQGISSALAWYQQKPGK
APKLLIYDASSLESGVPSRFSGSGSGTDFTLTISSLQPEDFA
TYYCQQFNSYPYTFGQGTKLEIK;
or
VH:
(SEQ ID NO: 3)
QVQLVESGGGVVQPGRSLRLSCAASGFTFSSYGFHWVRQAPG
KGLEWVAVIWYAGSNKFYADSVKGRFTISRDNSKNTLYLQMN
SLRAEDTAVYYCARGGQLDYYYYYVMDVWGQGTTVTVSS,
and
VL:
(SEQ ID NO: 4)
DIQMTQSPSSLSASVGDRVTITCRASQGISSWLAWYQQKPEK
APKSLIYAASSLQSGVPSRFSGSGSGTDFTLTISSLQPEDFA
TYYCQQYNSYPYTFGQGTKLEIK;
or
VH:
(SEQ ID NO: 5)
VQLVESGGGVVQPGRSLRLSCAASGFTFSSYGMHWVRQAPGK
GLEWVAVIWYAGSNKYYADSVKGRFTISRDNSKNTLYLQMNS
LRAEDTAVYYCARGGRIAVAFYYSMDVWGQGTTVTVSS,
and
VL:
(SEQ ID NO: 6)
DIQMTQSPSSLSASVGDRVTITCRASQGISSWLAWYQQKPEK
APKSLIYAASSLQSGVPSRFSGSGSGTDFTLTISSLQPEDFA
TYYCQQYNSYPYTFGQGTKLEIK.

In certain embodiments, an antibody comprising a pair of the above VH and VL light chains, or their CDRs, comprises a heavy chain constant region of an IgG1 isotype, either wild type or mutated, e.g., to be effectorless. In one embodiment, an anti-GITR antibody comprises the following heavy and light chains amino acid sequences:

heavy chain:
(SEQ ID NO: 7)
QVQLVESGGGVVQPGRSLRLSCAASGFTFSSYGMHWVRQ
APGKGLEWVAVIWYEGSNKYYADSVKGRFTISRDNSKNT
LYLQMNSLRAEDTAVYYCARGGSMVRGDYYYGMDVWGQG
TTVTVSSASTKGPSVFPLAPCSRSTSESTAALGCLVKDY
FPEPVTVSWNSGALTSGVHTFPAVLQSSGLYSLSSVVTV
PSSNFGTQTYTCNVDHKPSNTKVDKTVERKCCVECPPCP
APPVAGPSVFLFPPKPKDTLMISRTPEVTCVVVDVSHED
PEVQFNWYVDGVEVHNAKTKPREEQFNSTFRVVSVLTVV
HQDWLNGKEYKCKVSNKGLPAPIEKTISKTKGQPREPQV
YTLPPSREEMTKNQVSLTCLVKGFYPSDIAVEWESNGQP
ENNYKTTPPMLDSDGSFFLYSKLTVDKSRWQQGNVFSCS
VMHEALHNHYTQKSLSLSPG,
and
light chain:
(SEQ ID NO: 8)
AIQLTQSPSSLSASVGDRVTITCRASQGISSALAWYQQK
PGKAPKLLIYDASSLESGVPSRFSGSGSGTDFTLTISSL
QPEDFATYYCQQFNSYPYTFGQGTKLEIKRTVAAPSVFI
FPPSDEQLKSGTASVVCLLNNFYPREAKVQWKVDNALQS
GNSQESVTEQDSKDSTYSLSSTLTLSKADYEKHKVYACE
VTHQGLSSPVTKSFNRGEC,
or
heavy chain:
(SEQ ID NO: 9)
qvqlvesgggvvqpgrslrlscaasgftfssygmhwvrq
apgkglewvaviwyegsnkyyadsvkgrftisrdnsknt
lylqmnslraedtavyycarggsmvrgdyyygmdvwgqg
ttvtvssastkgpsvfplapsskstsggtaalgclvkdy
fpepvtvswnsgaltsgvhtfpavlgssglyslssvvtv
pssslgtqtyicnvnhkpsntkvdkrvepkscdkthtcp
pcpapeaegapsvflfppkpkdtlmisrtpevtcvvvdv
shedpevkfnwyvdgvevhnaktkpreegynstyrvvsv
ltvlhqdwingkeykckvsnkalpssiektiskakgqpr
epqvytlppsreemtknqvsltclvkgfypsdiavewes
nggpennykttppvldsdgsfflyskltvdksrwqqgnv
fscsvmhealhnhytqkslslspg,
and
light chain:
(SEQ ID NO: 10)
AIQLTQSPSSLSASVGDRVTITCRASQGISSALAWYQQK
PGKAPKLLIYDASSLESGVPSRFSGSGSGTDFTLTISSL
QPEDFATYYCQQFNSYPYTFGQGTKLEIKRTVAAPSVFI
FPPSDEQLKSGTASVVCLLNNFYPREAKVQWKVDNALQS
GNSQESVTEQDSKDSTYSLSSTLTLSKADYEKHKVYACE
VTHQGLSSPVTKSFNRGEC.

In certain embodiments, the anti-GITR antibody cross-competes with an anti-GITR antibody described herein, e.g., TRX518, MK4166 or an antibody comprising a VH domain and a VL domain amino acid sequence described herein. In some embodiments, the anti-GITR antibody binds the same epitope as that of an anti-GITR antibody described herein, e.g., TRX518, MK4166 or an antibody comprising a VH domain and a VL domain amino acid sequence described herein. In certain embodiments, the anti-GITR antibody comprises the six CDRs of TRX518, MK4166 or those of an antibody comprising a VH domain and a VL domain amino acid sequence described herein.

### Additional Antibodies

In some embodiments, the immunotherapy comprises an anti-TGFβ antibody. In certain embodiments, the anti-TGFβantibody is an anti-TGFβ antibody disclosed in Int'l Publ. No. WO/2009/073533.

In some embodiments, the immunotherapy comprises an anti-IL-10 antibody. In certain embodiments, the anti-IL-10 antibody is an anti-IL-10 antibody disclosed in Int'l Publ. No. WO/2009/073533.

In some other embodiments, the immunotherapy comprises an anti-B7-H4 antibody. In certain embodiments, the anti-B7-H4 antibody is an anti-B7-H4 antibody disclosed in Int'l Publ. No. WO/2009/073533.

In certain embodiments, the immunotherapy comprises an anti-Fas ligand antibody. In certain embodiments, the anti-Fas ligand antibody is an anti-Fas ligand antibody disclosed in Int'l Publ. No. WO/2009/073533.

In some embodiments, the immunotherapy comprises an anti-CXCR4 antibody. In certain embodiments, the anti-CXCR4 antibody is an anti-CXCR4 antibody disclosed in U.S. Publ. No. 2014/0322208 (e.g., Ulocuplumab (BMS-936564)).

In some embodiments is the immunotherapy comprises an anti-mesothelin antibody. In certain embodiments, the anti-mesothelin antibody is an anti-mesothelin antibody disclosed in U.S. Pat. No. 8,399,623.

In some embodiments, the immunotherapy comprises an anti-HER2 antibody. In certain embodiments, the anti-HER2 antibody is Herceptin (U.S. Pat. No. 5,821,337), trastuzumab, or ado-trastuzumab emtansine (Kadcyla, e.g., WO/2001/000244).

In embodiments, the immunotherapy comprises an anti-CD27 antibody. In embodiments, the anti-CD-27 antibody is Varlilumab (also known as “CDX-1127” and “1F5”), which is a human IgG1 antibody that is an agonist for human CD27, as disclosed in, for example, U.S. Pat. No. 9,169,325.

In some embodiments, the immunotherapy comprises an anti-CD73 antibody. In certain embodiments, the anti-CD73 antibody is CD73.4.IgG2C219S.IgG1.1f.

In some embodiments, the immunotherapy comprises an anti-MICA antibody. As used herein, an anti-MICA antibody is an antibody or an antigen binding fragment thereof that specifically binds MHC class I polypeptide-related sequence A. In some embodiments, the anti-MICA antibody binds MICB in addition to MICA. In some embodiments, the anti-MICA antibody inhibits cleavage of membrane bound MICA and release of soluble MICA. In certain embodiments, the anti-MICA antibody is an anti-MICA antibody disclosed in U.S. Publ. No. 2014/004112 A1, U.S. Publ. No. 2016/046716 A1, or U.S. Publ. No. 2017/022275 A1.

In some embodiments, the immunotherapy comprises an anti-TIM3 antibody. As used herein, an anti-TIM3 antibody is an antibody or an antigen binding fragment thereof that specifically binds T-cell immunoglobulin and mucin-domain containing-3 (TIM3), also known as hepatitis A virus cellular receptor 2 (HAVCR2). In some embodiments, the anti-TIM3 antibody is capable of stimulating an immune response, e.g., an antigen-specific T cell response. In some embodiments, the anti-TIM3 antibody binds to soluble or membrane bound human or cyno TIM3. In certain embodiments, the anti-TIM3 antibody is an anti-TIM3 antibody disclosed in International Publication No. WO/2018/013818, which is incorporated by reference herein in its entirety.

In some embodiments, the method comprises administering a combination therapy comprising two or more antibodies. In some embodiments, the two or more antibodies are selected from the group consisting of PD-1, PD-L1, CTLA-4, LAG3, TIGIT, TIM3, NKG2a, OX40, ICOS, MICA, CD137, KIR, TGFβ, IL-10, IL-8, B7-H4, Fas ligand, CXCR4, mesothelin, CD27, GITR. In certain embodiments, the combination therapy comprises administering a combination of an anti-PD-1 antibody and an anti-CTLA-4 antibody. In some embodiments, the combination therapy comprises administering a combination of an anti-PD-L1 antibody and an anti-CTLA-4 antibody. In some embodiments, the combination therapy comprises administering a combination of an anti-PD-L1 antibody and an anti-LAG3 antibody. In some embodiments, the combination therapy comprises administering a combination of an anti-PD-L1 antibody and an anti-TIM3 antibody. In some embodiments, the combination therapy comprises administering a combination of an anti-PD-L1 antibody and an anti-GITR antibody. In some embodiments, the combination therapy comprises administering a combination of an anti-PD-L1 antibody and an anti-MICA antibody. In some embodiments, the combination therapy comprises administering a combination of an anti-PD-L1 antibody and an anti-CD137 antibody. In some embodiments, the combination therapy comprises administering a combination of an anti-PD-L1 antibody and an anti-CD27 antibody. In some embodiments, the combination therapy comprises administering a combination of an anti-PD-L1 antibody and an anti-CXCR4 antibody.

### Cytokines

In some embodiments, the method comprises administering a combination therapy comprising an antibody and a cytokine. The cytokine can be any cytokine or variant thereof known in the art. In some embodiments, the cytokine is selected from the group consisting of interleukin-2 (IL-2), IL-1β, IL-6, TNF-α, RANTES, monocyte chemoattractant protein (MCP-1), monocyte inflammatory protein (MIP-1α and MIP-1β), IL-8, lymphotactin, fractalkine, IL-1, IL-4, IL-10, IL-11, IL-13, LIF, interferon-alpha, TGF-beta, and any combination thereof. In some embodiments, the cytokine is a CD122 agonist. In certain embodiments, the cytokine comprises IL-2 or a variant thereof.

In some embodiments, the cytokine comprises one or more amino acid substitution, deletion, or insertion relative to the wild-type cytokine amino acid sequence. In some embodiments, the cytokine comprises an amino acid sequence having at least 1, at least 2, at least 3, at least 4, at least 5, at least 6, at least 7, at least 8, at least 9, or at least 10 amino acids substituted relative to the amino acid sequence of the wild-type cytokine.

In some embodiments, the cytokine is modified, e.g., to increase activity and/or half-life. In certain embodiments, the cytokine is modified through fusion of a heterologous moiety to the cytokine. The heterologous moiety can be any structure including a polypeptide, a polymer, a small molecule, a nucleotide, or a fragment or analog thereof. In certain embodiments, the heterologous moiety comprises a polypeptide. In some embodiments, the heterologous moiety comprises albumin or a fragment thereof, albumin-binding polypeptide (ABP), XTEN, Fc, PAS, the C-terminal peptide (CTP) of the β subunit of human chorionic gonadotropin, or any combination thereof.

In certain embodiments, the cytokine is modified through fusion of the cytokine with a polymer. In some embodiments, the polymer comprises polyethylene glycol (PEG), polypropylene glycol (PPG), hydroxyethyl starch (HES), or any combination thereof. “PEG” or “polyethylene glycol,” as used herein, is meant to encompass any water-soluble poly (ethylene oxide). Unless otherwise indicated, a “PEG polymer” or a polyethylene glycol is one in which substantially all (preferably all) monomeric subunits are ethylene oxide subunits, though, the polymer may contain distinct end capping moieties or functional groups, e.g., for conjugation. PEG polymers for use in the present disclosure will comprise one of the two following structures: “—(CH₂CH₂O)ₙ₋ₙ or “—(CH₂CH₂O)ₙ₋₁CH₂CH₂—,” depending upon whether or not the terminal oxygen(s) has been displaced, e.g., during a synthetic transformation. As stated above, for the PEG polymers, the variable (n) ranges from about 3 to 4000, and the terminal groups and architecture of the overall PEG can vary.

In some embodiments, the methods of the present disclosure comprising administering to a subject having a high TMB status an immunotherapy, wherein the immunotherapy comprises an antibody and a CD122 agonist. In some embodiments, the immunotherapy comprises administering (1) an anti-PD-1 antibody, an anti-CTLA-4 antibody, an anti-CTLA-4 antibody, or any combination thereof and (2) a CD122 agonist. In some embodiments, the CD122 agonist comprises IL-2 or a variant thereof. In some embodiments, the CD122 agonist comprises an IL-2 variant having at least 1 amino acid substitution relative to wild-type IL-2. In some embodiments, the CD122 agonist comprises an IL-2 fused to a PEG. In some embodiments, the CD122 agonist comprises an IL-2 variant having at least 1 amino acid substitution relative to wild-type IL-2, wherein the IL-2 variant is fused to a PEG.

### Standard-of-Care Therapies for Cancer

In some embodiments, the methods disclosed herein are used in place of standard of care therapies. In certain embodiments, a standard of care therapy is used in combination with any method disclosed herein. Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers (see NCCN GUIDELINES®, 2014).

### Colorectal Cancer

In some embodiments, the combination therapy treats a cancer, which is colorectal cancer. In embodiments, the colorectal cancer is colon cancer. In other embodiments, the colorectal cancer is rectal cancer. In certain embodiments, the colorectal cancer has microsatellite instability (MSI). (See Pawlik et al., Dis. Markers 20 (4-5): 199-206 (2004)) In other embodiments, the colorectal cancer has low microsatellite instability (MSI-L).

Colorectal cancer is the third most common type of cancer in both men and women in the U.S. (See http://www.cancer.gov/types/colorectal, last visited Dec. 9, 2015). Most colorectal cancers are adenocarcinomas. Colon cancer presents in five stages: Stage 0 (Carcinoma in Situ), Stage I, Stage II, Stage III and Stage IV. Six types of standard treatment are used for colon cancer: 1) surgery, including a local excision, resection of the colon with anastomosis, or resection of the colon with colostomy; 2) radiofrequency ablation; 3) cryosurgery; 4) chemotherapy; 5) radiation therapy; and 6) targeted therapies, including monoclonal antibodies and angiogenesis inhibitors. In some embodiments, the combination therapy of the disclosure treats a colon cancer along with a standard of care therapy.

Rectal cancer presents in five stages: Stage 0 (Carcinoma in Situ), Stage I, Stage II, Stage III and Stage IV. Six types of standard treatment are used for rectal cancer: 1) Surgery, including polypectomy, local excision, resection, radiofrequency ablation, cryosurgery, and pelvic exenteration; 2) radiation therapy; 3) chemotherapy; and 4) targeted therapy, including monoclonal antibody therapy. In some embodiments, the methods of the disclosure treat a rectal cancer along with a standard of care therapy.

### Lung Cancer

In some embodiments, the combination therapy of the disclosure treats a cancer, which is lung cancer. In certain embodiments the cancer is NSCLC. In embodiments, the NSCLC has a squamous histology. In other embodiments, the NSCLC has a nonsquamous histology.

NSCLC is the leading cause of cancer death in the U.S. and worldwide, exceeding breast, colon and prostate cancer combined. In the U.S., an estimated 228,190 new cases of lung and bronchial will be diagnosed in the U.S., and some 159,480 deaths will occur because of the disease (Siegel et al. (2014) CA Cancer J Clin 64 (1): 9-29). The majority of patients (approximately 78%) are diagnosed with advanced/recurrent or metastatic disease. Metastases to the adrenal gland from lung cancer are a common occurrence, with about 33% of patients having such metastases. NSCLC therapies have incrementally improved OS, but benefit has reached a plateau (median OS for late stage patients is just 1 year). Progression after 1L therapy occurred in nearly all of these subjects and the 5-year survival rate is only 3.6% in the refractory setting. From 2005 to 2009, the overall 5-year relative survival rate for lung cancer in the U.S. was 15.9% (NCCN GUIDELINES®, Version 3.2014-Non-Small Cell Lung Cancer, available at: www.nccn.org/professionals/physician_gls/pdf/nscl.pdf, last accessed May 14, 2014).

There are seven stages of NSCLC: Occult non-small cell lung cancer, Stage 0 (carcinoma in situ), Stage I, Stage II, Stage IIIA, Stage IIIB, and Stage IV. In some embodiments, the combination therapy of the disclosure treats a NSCLC along with a standard of care therapy.

In addition, the present methods can also be combined with surgery, radiation therapy (RT) and chemotherapy that are the three modalities commonly used to treat NSCLC patients. As a class, NSCLCs are relatively insensitive to chemotherapy and RT, compared to small cell carcinoma. In general, for patients with Stage I or II disease, surgical resection provides the best chance for cure, with chemotherapy increasingly being used both pre-operatively and post-operatively. RT can also be used as adjuvant therapy for patients with resectable NSCLC, the primary local treatment, or as palliative therapy for patients with incurable NSCLC.

In one embodiment, the subject suitable for the methods of the present disclosure is a patient with Stage IV disease. Patients with Stage IV disease have a good performance status (PS) benefit from chemotherapy. Many drugs, including platinum agents (e.g., cisplatin, carboplatin), taxanes agents (e.g., paclitaxel, albumin-bound paclitaxel, and docetaxel), vinorelbine, vinblastine, etoposide, pemetrexed and gemcitabine are useful for Stage IV NSCLC. Combinations using many of these drugs produce 1-year survival rates of 30% to 40% and are superior to single agents. Specific targeted therapies have also been developed for the treatment of advanced lung cancer. For example, bevacizumab (AVASTIN®) is a monoclonal antibody that blocks vascular endothelial growth factor A (VEGF-A). Erlotinib (TARCEVA®) is a small-molecule TKI of epidermal growth factor receptor (EGFR). Crizotinib (XALKORI®) is a small-molecule TKI that targets ALK and MET, and is used to treat NSCLC in patients carrying the mutated ALK fusion gene. Cetuximab (ERBITUX®) is a monoclonal antibody that targets EGFR.

In some embodiments, the present methods are used to treat a subject who has squamous NSCLC. In certain embodiments, the present methods are used in combination with a standard of care therapy. There is a particular unmet need among patients who have squamous cell NSCLC (representing up to 25% of all NSCLC) as there are few treatment options after first line (IL) therapy. Single-agent chemotherapy is standard of care following progression with platinum-based doublet chemotherapy (Pt-doublet), resulting in median OS of approximately 7 months. Docetaxel remains the benchmark treatment in this line of therapy although erlotinib can also be used with less frequency. Pemetrexed has also been shown to produce clinically equivalent efficacy outcomes but with significantly fewer side effects compared with docetaxel in the second line (2L) treatment of patients with advanced NSCLC (Hanna et al., 2004 J Clin Oncol 22:1589-97). No therapy is currently approved for use in lung cancer beyond the third line (3L) setting. Pemetrexed and bevacizumab are not approved in squamous NSCLC, and molecularly targeted therapies have limited application. The unmet need in advanced lung cancer has been compounded by the recent failure of Oncothyreon and Merck KgaA's STIMUVAX® to improve OS in a phase 3 trial, inability of ArQule's and Daiichi Sankyo's c-Met kinase inhibitor, tivantinib, to meet survival endpoints, failure of Eli Lilly's ALIMTA® in combination with Roche's AVASTIN® to improve OS in a late-stage study, and Amgen's and Takeda Pharmaceutical's failure to meet clinical endpoints with the small-molecule VEGF-R antagonist, motesanib, in late-stage trials.

### Combination Therapies

Certain aspects of the present disclosure are directed to methods for treating a subject afflicted with a tumor comprising administering to the subject a therapeutically effective amount of (a) an anti-PD-1 antibody or an anti-PD-L1 antibody and (b) an antibody or antigen-binding portion thereof that binds specifically to cytotoxic T-lymphocyte-associated protein 4 (CTLA-4) (“an anti-CTLA-4 antibody”), wherein the tumor has a high tumor mutation burden (TMB) status. In certain embodiments, the tumor is derived from a non-small cell lung cancer (NSCLC). In some embodiment, the high TMB is characterized by at least about 10 mutations per megabase of genes examined. In particular embodiments, the method further comprises measuring the TMB stratus of a biological sample obtained from the subject prior to the administering.

In certain embodiments, the anti-PD-1 antibody, the anti-PD-L1 antibody, and/or the anti-CTLA-4 antibody are administered at a therapeutically effective amount. In some embodiments, the method comprises administering a therapeutically effective amount of anti-PD-1 antibody and an anti-CTLA-4 antibody. In other embodiments, the method comprises administering a therapeutically effective amount of anti-PD-L1 antibody and an anti-CTLA-4 antibody. Any anti-PD-1, anti-PD-L1, or anti-CTLA-4 antibody disclosed herein can be used in the method. In certain embodiments, the anti-PD-1 antibody comprises nivolumab. In some embodiments, the anti-PD-1 antibody comprises pembrolizumab. In some embodiments, the anti-PD-L1 antibody comprises atezolizumab. In some embodiments, the anti-PD-L1 antibody comprises durvalumab. In some embodiments, the anti-PD-L1 antibody comprises avelumab. In some embodiments, the anti-CTLA-4 antibody comprises ipilimumab. In some embodiments, the anti-CTLA-4 antibody comprises ipilimumab tremelimumab.

In some embodiments, the anti-PD-1 antibody or the anti-PD-L1 antibody and the anti-CTLA-4 antibody are each administered once about every 2 weeks, once about every 3 weeks, once about every 4 weeks, once about every 5 weeks, or once about every 6 weeks. In some embodiments, the anti-PD-1 antibody or the anti-PD-L1 antibody is administered once about every 2 weeks, once about every 3 weeks or once about every 4 weeks, and the anti-CTLA-4 antibody is administered once about every 6 weeks.

In some embodiments, the anti-CTLA-4 antibody is administered at a dose ranging from about 0.1 mg/kg to about 20.0 mg/kg body weight once about every 2, 3, 4, 5, 6, 7, or 8 weeks. In some embodiments, the anti-CTLA-4 antibody is administered at a dose of about 0.1 mg/kg, about 0.3 mg/kg, about 0.6 mg/kg, about 0.9 mg/kg, about 1 mg/kg, about 3 mg/kg, about 6 mg/kg, about 9 mg/kg, about 10 mg/kg, about 12 mg/kg, about 15 mg/kg, about 18 mg/kg, or about 20 mg/kg. In certain embodiments, the anti-CTLA-4 antibody is administered at a dose of about 1 mg/kg once about every 4 weeks. In some embodiments, the anti-CTLA-4 antibody is administered at a dose of about 1 mg/kg once about every 6 weeks.

In some embodiments, the anti-CTLA-4 antibody is administered at a flat dose. In some embodiments, the anti-CTLA-4 antibody is administered at a flat dose ranging from at least about 40 mg to at least about 1600 mg. In some embodiments, the anti-CTLA-4 antibody is administered at a flat dose of at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, or at least about 200 mg. In some embodiments, the CTLA-4 antibody is administered at a flat dose of at least about 220 mg, at least about 230 mg, at least about 240 mg, at least about 250 mg, at least about 260 mg, at least about 270 mg, at least about 280 mg, at least about 290 mg, at least about 300 mg, at least about 320 mg, at least about 360 mg, at least about 400 mg, at least about 440 mg, at least about 480 mg, at least about 520 mg, at least about 560 mg, or at least about 600 mg. In some embodiments, the CTLA-4 antibody is administered at a flat dose of at least about 640 mg, at least about 720 mg, at least about 800 mg, at least about 880 mg, at least about 960 mg, at least about 1040 mg, at least about 1120 mg, at least about 1200 mg, at least about 1280 mg, at least about 1360 mg, at least about 1440 mg, or at least about 1600 mg. In some embodiments, the anti-CTLA-4 antibody is administered in a flat dose at least once about every 2, 3, 4, 5, 6, 7, or 8 weeks.

In certain embodiments, the anti-PD-1 antibody is administered at a dose of about 2 mg/kg once about every 3 weeks and the anti-CTLA-4 antibody is administered at a dose of about 1 mg/kg once about every 6 weeks. In some embodiments, the anti-PD-1 antibody is administered at a dose of about 3 mg/kg once about every 2 weeks and the anti-CTLA-4 antibody is administered at a dose of about 1 mg/kg once about every 6 weeks. In some embodiments, the anti-PD-1 antibody is administered at a dose of about 6 mg/kg once about every 4 weeks and the anti-CTLA-4 antibody is administered at a dose of about 1 mg/kg once about every 6 weeks.

In certain embodiments, the anti-PD-1 antibody is administered at a flat dose of about 200 mg once about every 3 weeks and the anti-CTLA-4 antibody is administered at a dose of about 1 mg/kg once about every 6 weeks. In some embodiments, the anti-PD-1 antibody is administered at a flat dose of about 240 mg once about every 2 weeks and the anti-CTLA-4 antibody is administered at a dose of about 1 mg/kg once about every 6 weeks. In some embodiments, the anti-PD-1 antibody is administered at a flat dose of about 480 mg once about every 4 weeks and the anti-CTLA-4 antibody is administered at a dose of about 1 mg/kg once about every 6 weeks.

In certain embodiments, the anti-PD-1 antibody is administered at a flat dose of about 200 mg once about every 3 weeks and the anti-CTLA-4 antibody is administered at a flat dose of about 80 mg once about every 6 weeks. In some embodiments, the anti-PD-1 antibody is administered at a flat dose of about 240 mg once about every 2 weeks and the anti-CTLA-4 antibody is administered at a dose of about 80 mg once about every 6 weeks. In some embodiments, the anti-PD-1 antibody is administered at a flat dose of about 480 mg once about every 4 weeks and the anti-CTLA-4 antibody is administered at a dose of about 80 mg once about every 6 weeks.

In certain embodiments, the anti-PD-L1 antibody is administered at a dose of about 10 mg/kg once about every 2 weeks and the anti-CTLA-4 antibody is administered at a dose of about 1 mg/kg once about every 6 weeks. In some embodiments, the anti-PD-L1 antibody is administered at a dose of about 15 mg/kg once about every 3 weeks and the anti-CTLA-4 antibody is administered at a dose of about 1 mg/kg once about every 6 weeks.

In certain embodiments, the anti-PD-L1 antibody is administered at a flat dose of about 800 mg once about every 2 weeks and the anti-CTLA-4 antibody is administered at a dose of about 1 mg/kg once about every 6 weeks. In some embodiments, the anti-PD-L1 antibody is administered at a flat dose of about 1200 mg once about every 3 weeks and the anti-CTLA-4 antibody is administered at a dose of about 1 mg/kg once about every 6 weeks.

In certain embodiments, the anti-PD-L1 antibody is administered at a flat dose of about 800 mg once about every 2 weeks and the anti-CTLA-4 antibody is administered at a flat dose of about 80 mg once about every 6 weeks. In some embodiments, the anti-PD-L1 antibody is administered at a flat dose of about 1200 mg once about every 3 weeks and the anti-CTLA-4 antibody is administered at a dose of about 80 mg once about every 6 weeks.

### Melanoma

In some embodiments, the combination therapy treats a cancer, which is melanoma. Melanoma is the most deadly form of skin cancer, and is the fifth most common cancer diagnosis in men and the seventh most common cancer diagnosis in women. (See http://www.cancer.gov/types/skin, last visited Dec. 9, 2015). Melanoma presents in seven stages: Stage 0 (Melanoma in situ), Stage I, Stage II, Stage III that can be removed by surgery, Stage III that cannot be removed by surgery, Stage IV, and Recurrent Melanoma. Five standard types of treatment are used: 1) surgery; 2) chemotherapy; 3) radiation therapy and 4) biologic therapy, including interferon, interleukin-2 (IL-2), tumor necrosis factor (TNF) therapy, and ipilimumab, and 5) targeted therapy, including signal transduction inhibitor therapy (e.g., vemurafenib, dabrafenib, and trametinib), oncolytic virus therapy, monoclonal antibody therapy (including pembrolizumab and nivolumab), and angiogenesis inhibitors. In some embodiments, the combination therapy of the disclosure treats a melanoma along with a standard of care therapy

### Ovarian Cancer

In certain embodiments, the combination therapy treats a cancer, which is ovarian, fallopian tube and primary peritoneal cancer (“ovarian cancer”). In certain embodiments, the cancer is ovarian epithelial cancer. In other embodiments, the cancer is ovarian germ cell tumor. In yet other embodiments, the cancer is an ovarian low malignant potential tumor. In embodiments, the ovarian cancer begins in the tissue that covers the ovaries, the peritoneum or the fallopian tube. (See http://www.cancer.gov/types/ovarian/patient/ovarian-epithelial-treatment-pdq, last visited Dec. 9, 2015).

There are four stages of ovarian cancer: Stage I, Stage II, Stage III, and Stage IV, which encompass early, advanced and recurrent or persistent ovarian cancer. There are four types of standard treatments that are used for patients with ovarian, fallopian tube and primary peritoneal cancer: 1) surgery, including hysterectomy, unilateral salpingo-oophorectomy, bilateral salpingo-oophorectomy, omentectomy, and lymph node biopsy; 2) radiation therapy; 3) chemotherapy; and 4) targeted therapy, including monoclonal antibody therapy and poly (ADP-ribose) polymerase inhibitors. Biologic therapies are also being tested for ovarian cancer. In some embodiments, the combination therapy of the disclosure treats an ovarian cancer along with a standard of care therapy.

There are four stages of ovarian germ cell tumors: Stage I, Stage II, Stage III and Stage IV. Four types of standard treatment are used: 1) surgery, including unilateral salpingo-oophorectomy, total hysterectomy, bilateral salpingo-oophorectomy, and tumor debulking; 2) observation; 3) chemotherapy and 4) radiation therapy. New treatment options being considered include high-dose chemotherapy with bone marrow transplant. In some embodiments, the combination therapy of the disclosure treats an ovarian germ cell tumor along with a standard of care therapy.

There are 3 stages of ovarian low malignant potential tumors: 1) early stage (Stage I and II), 2) late stage (Stage III and IB) and 3) recurrent. Two types of standard treatment are used: 1) surgery, including unilateral salpingo-oophorectomy, bilateral salpingo-oophorectomy, total hysterectomy, partial oophorectomy, and omentectomy and 2) chemotherapy. In some embodiments, the combination therapy of the disclosure treats an ovarian low malignant potential tumor along with a standard of care therapy.

### Head and Neck Cancer

In some embodiments, the combination therapy treats a cancer, which is head and neck cancer. Head and neck cancers include cancers of the oral cavity, pharynx, larynx, paranasal sinuses and nasal cavity and salivary glands. Head and neck cancers usually begin in the squamous cells that line the moist, mucosal surfaces inside the head and neck (for example, inside the mouth, the nose, and the throat). These squamous cell cancers are often referred to as squamous cell carcinomas of the head and neck. Head and neck cancers can also begin in the salivary glands, but salivary gland cancers are relatively uncommon. (See http://www.cancer.gov/types/head-and-neck/head-neck-fact-sheet, last visited Dec. 9, 2015). The treatment plan for an individual patient depends on a number of factors, including the exact location of the tumor, the stage of the cancer, and the person's age and general health. Treatment for head and neck cancer can include surgery, radiation therapy, chemotherapy, targeted therapy, or a combination of treatments. In some embodiments, the combination therapy of the disclosure treats a head and neck cancer along with a standard of care therapy.

### Immunotherapy of Lung Cancer

A clear need exists for effective agents for patients who have progressed on multiple lines of targeted therapy, as well as for therapies that extend survival for longer periods beyond the current standard treatments. Newer approaches involving immunotherapy, especially blockade of immune checkpoints including the CTLA-4, PD-1, and PD-L1 inhibitory pathways, have recently shown promise (Creelan et al., 2014). Thus, ipilimumab in combination with chemotherapy has exhibited encouraging results in small-cell and non-small-cell lung cancer alike. Clinical trials of the monoclonal antibodies nivolumab, pembrolizumab, BMS-936559, MEDI4736, and MPDL3280A are demonstrating durable overall radiological response rates in the 20% to 25% range in lung cancer (Topalian et al, 2012a; Pardoll, 2012; WO 2013/173223; Creelan et al., 2014). This exceptional activity includes squamous lung cancers, a population historically bereft of significant therapeutic advances.

### Pharmaceutical Compositions and Dosages

Therapeutic agents of the present disclosure can be constituted in a composition, e.g., a pharmaceutical composition containing an antibody and/or a cytokine and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier for a composition containing an antibody is suitable for intravenous, intramuscular, subcutaneous, parenteral, spinal or epidermal administration (e.g., by injection or infusion), whereas the carrier for a composition containing an antibody and/or a cytokine is suitable for non-parenteral, e.g., oral, administration. In some embodiments, the subcutaneous injection is based on Halozyme Therapeutics' ENHANZE® drug-delivery technology (see U.S. Pat. No. 7,767,429, which is incorporated by reference herein in its entirety). ENHANZE® uses a co-formulation of an Ab with recombinant human hyaluronidase enzyme (rHuPH20), which removes traditional limitations on the volume of biologics and drugs that can be delivered subcutaneously due to the extracellular matrix (see U.S. Pat. No. 7,767,429). A pharmaceutical composition of the disclosure can include one or more pharmaceutically acceptable salts, anti-oxidant, aqueous and non-aqueous carriers, and/or adjuvants such as preservatives, wetting agents, emulsifying agents and dispersing agents. Therefore, in some embodiments, the pharmaceutical composition for the present disclosure can further comprise recombinant human hyaluronidase enzyme, e.g., rHuPH20.

Dosage regimens are adjusted to provide the optimum desired response, e.g., a maximal therapeutic response and/or minimal adverse effects. In some embodiments, the anti-PD-1 antibody or an anti-PD-L1 antibody is administered at a weight-based dose. For administration of an anti-PD-1 antibody or an anti-PD-L1 antibody, especially in combination with another anti-cancer agent, the dosage can range from about 0.01 to about 20 mg/kg, from about 0.1 to about 10 mg/kg, from about 0.01 to about 5 mg/kg, from about 1 to about 5 mg/kg, from about 2 to about 5 mg/kg, from about 1 to about 3 mg/kg, from about 7.5 to about 12.5 mg/kg, or from about 0.1 to about 30 mg/kg of the subject's body weight. For example, dosages can be about 0.1, about 0.3, about 1, about 2, about 3, about 5, or about 10 mg/kg body weight, and more preferably, 0.3, 1, 2, 3, or 5 mg/kg body weight. In certain embodiments, the dosage of the anti-PD-1 antibody is 3 mg/kg body weight.

In one embodiment, a dosage regimen for an anti-PD-1 antibody or an anti-PD-L1 antibody of the disclosure comprises about 0.3-1 mg/kg body weight, about 5 mg/kg body weight, 1-5 mg/kg body weight, or about 1-about 3 mg/kg body weight via intravenous administration, with the antibody being given every about 14-21 days in up to about 6-week or about 12-week cycles until complete response or confirmed progressive disease. In some embodiments, the antibody treatment, or any combination treatment disclosed herein, is continued for at least about 1 month, at least about 3 months, at least about 6 months, at least about 9 months, at least about 1 year, at least about 18 months, at least about 24 months, at least about 3 years, at least about 5 years, or at least about 10 years.

The dosing schedule is typically designed to achieve exposures that result in sustained receptor occupancy (RO) based on typical pharmacokinetic properties of an antibody. An exemplary treatment regime entails administration once per week, once every 2 weeks, once every 3 weeks, once every 4 weeks, once a month, once every 3-6 months or longer. In certain preferred embodiments, an anti-PD-1 antibody such as nivolumab is administered to the subject once every 2 weeks. In other preferred embodiments, the antibody is administered once every 3 weeks. The anti-PD-1 antibody can be administered in at least two doses, each of the doses is at an amount of about 0.01 mg/kg to about 5 mg/kg, e.g., 3 mg/kg, at a dosing interval of every two weeks between the two doses. In some embodiments, the anti-PD-1 antibody is administered in at least three, four, five, six, or seven doses (i.e., multiple doses), each of the doses is at an amount of about 0.01 mg/kg to about 5 mg/kg, e.g., 3 mg/kg, at a dosing interval of every two weeks between two adjacently given doses. The dosage and scheduling can change during a course of treatment. For example, a dosing schedule for anti-PD-1 monotherapy can comprise administering the antibody: (i) every 2 weeks in 6-week cycles; (ii) every 4 weeks for six dosages, then every three months; (iii) every 3 weeks; or (iv) 3-10 mg/kg once followed by 1 mg/kg every 2-3 weeks. Considering that an IgG4 antibody typically has a half-life of 2-3 weeks, a preferred dosage regimen for an anti-PD-1 antibody of the disclosure comprises 0.3-10 mg/kg body weight, preferably 1-5 mg/kg body weight, more preferably 1-3 mg/kg body weight via intravenous administration, with the antibody being given every 14-21 days in up to 6-week or 12-week cycles until complete response or confirmed progressive disease.

In certain embodiments, an anti-PD-1 antibody or an anti-PD-L1 antibody is administered at a flat dose. In embodiments, the anti-PD-1 antibody or an anti-PD-L1 antibody is administered at a flat dose as a monotherapy. In embodiments, the anti-PD-1 antibody or an anti-PD-L1 antibody is administered as a flat dose in combination with any other therapy disclosed herein. In embodiments, the flat dose of the anti-PD-1 antibody or an anti-PD-L1 antibody is a dose of at least about 100-600 mg, such as, at least about 200-300 mg, at least about 400-500 mg, or at least about 240 mg or at least about 480 mg, such as at least about 60 mg, at least about 80 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, at least about 320 mg, at least about 360 mg, at least about 400 mg, at least about 440 mg, at least about 480 mg, at least about 520 mg, at least bout 560 mg, at least about 600 mg, or at least about 660 mg, or at least about 720 mg. In some embodiments, the flat dose of the anti-PD-1 antibody or an anti-PD-L1 antibody is a dose of at least about 600-1200 mg. In some embodiments, flat dose of the anti-PD-1 antibody or an anti-PD-L1 antibody is a dose of at least about 600 mg, at least about 640 mg, at least about 680 mg, at least about 720 mg, at least about 760 mg, at least about 800 mg, at least about 840 mg, at least about 880 mg, at least about 920 mg, at least about 960 mg, at least about 1000 mg, at least about 1040 mg, at least about 1080 mg, at least about 1120 mg, at least about 1160 mg, or at least about 1200 mg. In some embodiments, the anti-PD-1 antibody or antigen-binding portion thereof is administered at a dose of at least about 240 mg or at least about 480 mg once about every 2 or 4 weeks. In some embodiments, the anti-PD-L1 antibody or antigen-binding portion thereof is administered at a dose of at least about 240 mg or at least about 480 mg once about every 2 or 4 weeks. In some embodiments, the anti-PD-1 antibody or the anti-PD-L1 antibody is administered at a dose of at least about 720 mg. In some embodiments, the anti-PD-1 antibody or the anti-PD-L1 antibody is administered at a dose of at least about 960 mg. In some embodiments, the anti-PD-1 antibody or the anti-PD-L1 antibody is administered at a dose of at least about 1200 mg.

In other embodiments, the anti-PD-1 antibody or antigen-binding portion thereof is administered at a dose higher than, i.e., at least about, 240 mg. When used in combinations with other cancer agents, the dosage of an anti-PD-1 antibody can be lowered compared to the monotherapy dose. For example, a dosage of nivolumab that is significantly lower than the typical 3 mg/kg every 3 weeks, for instance 0.1 mg/kg or less every 3 or 4 weeks, is regarded as a subtherapeutic dosage. Receptor-occupancy data from 15 subjects who received 0.3 mg/kg to 10 mg/kg dosing with nivolumab indicate that PD-1 occupancy appears to be dose-independent in this dose range. Across all doses, the mean occupancy rate was 85% (range, 70% to 97%), with a mean plateau occupancy of 72% (range, 59% to 81%) (Brahmer et al., J Clin Oncol 28:3167-75 2010). Thus, 0.3 mg/kg dosing can allow for sufficient exposure to lead to maximal biologic activity.

In some embodiments, the anti-PD-1 antibody or the anti-PD-L1 antibody is administered in a fixed dose with a second agent. In some embodiments, the anti-PD-1 antibody is administered in a fixed dose with a second immunotherapeutic agent. In some embodiments, the ratio of the anti-PD-1 antibody or the anti-PD-L1 antibody to the second agent, e.g., the second immunotherapeutic agent, is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg.

Although higher nivolumab monotherapy dosing up to 10 mg/kg every two weeks has been achieved without reaching the maximum tolerated does (MTD), the significant toxicities reported in other trials of checkpoint inhibitors plus anti-angiogenic therapy (see, e.g., Johnson et al., 2013; Rini et al., 2011) support the selection of a nivolumab dose lower than 10 mg/kg.

For combination of nivolumab with other anti-cancer agents, these agents are preferably administered at their approved dosages. Treatment is continued as long as clinical benefit is observed or until unacceptable toxicity or disease progression occurs. Nevertheless, in certain embodiments, the dosages of these anti-cancer agents administered are significantly lower than the approved dosage, i.e., a subtherapeutic dosage, of the agent is administered in combination with the anti-PD-1 antibody or an anti-PD-L1 antibody. The anti-PD-1 antibody or anti-PD-L1 antibody can be administered at the dosage that has been shown to produce the highest efficacy as monotherapy in clinical trials, e.g., about 3 mg/kg of nivolumab administered once every three weeks (Topalian et al., 2012a; Topalian et al., 2012), or at a significantly lower dose, i.e., at a subtherapeutic dose.

Dosage and frequency vary depending on the half-life of the antibody in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

### Kits

Also within the scope of the present disclosure are kits comprising an immunotherapy, e.g., an anti-PD-1 antibody for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a tumor, the kit comprising: (a) a dosage ranging from 0.1 to 10 mg/kg body weight of an antibody or an antigen-binding portion thereof that specifically binds to the PD-1 receptor and inhibits PD-1 activity (“an anti-PD-1 antibody”); and (b) instructions for using the anti-PD-1 antibody in the methods disclosed herein. In certain embodiments, the tumor is lung cancer, e.g., NSCLC. In certain preferred embodiments for treating human patients, the kit comprises an anti-human PD-1 antibody disclosed herein, e.g., nivolumab or pembrolizumab.

In some embodiments, the kit further includes a comprehensive genomic profiling assay disclosed herein. In some embodiments, the kit further includes instructions to administer the immunotherapy, e.g., the anti-PD-1 antibody, the anti-PD-L1 antibody, the anti-CTLA-4 antibody, and or the cytokine, to a subject identified as having a high TMB status, according to the methods disclosed herein.

All of the references cited above, as well as all references cited herein, are incorporated herein by reference in their entireties.

The following examples are offered by way of illustration and not by way of limitation.

### EXAMPLES

### Example 1

### A Phase 3 Study of First-Line Nivolumab in Stage IV or Recurrent Non-Small-Cell Lung Cancer

### Overview

Nivolumab improves overall survival (OS) versus docetaxel in previously treated non-small-cell lung cancer (NSCLC). This open-label phase 3 study compared first-line nivolumab versus chemotherapy in programmed death-ligand 1 (PD-L1)-positive NSCLC.

Patients with untreated stage IV/recurrent NSCLC and ≥1% PD-L1 tumor expression were randomized 1:1 to nivolumab 3 mg/kg once every 2 weeks or platinum-based chemotherapy. The primary endpoint was progression-free survival (PFS) per blinded independent central review in patients with ≥5% PD-L1 expression.

In patients with ≥5% PD-L1 expression (n=423), median PFS was 4.2 months with nivolumab versus 5.9 months with chemotherapy (hazard ratio [HR], 1.15; 95% confidence interval [CI], 0.91 to 1.45; P=0.2511); median OS was 14.4 versus 13.2 months (HR, 1.02; 95% CI, 0.80 to 1.30); 128 (60%) patients randomized to chemotherapy received subsequent nivolumab. In patients with high tumor mutation burden (TMB; upper tertile), nivolumab improved PFS (HR, 0.62; 95% CI, 0.38 to 1.00) and objective response rate (ORR; 46.8% vs. 28.3%) versus chemotherapy. Any grade and grade 3/4 treatment-related adverse events occurred in 71% and 18% of nivolumab-treated and 92% and 51% of chemotherapy-treated patients, respectively.

Nivolumab did not show superior PFS versus chemotherapy in previously untreated stage IV/recurrent NSCLC with ≥5% PD-L1 expression; OS was similar between arms. Nivolumab had a favorable safety profile versus chemotherapy. In this first phase 3 trial incorporating an analysis of TMB and clinical benefit with a PD-1/L1 inhibitor, findings suggest that nivolumab improves ORR and PFS versus chemotherapy in patients with high TMB.

For the past two decades, platinum-based combination chemotherapy has been standard-of-care first-line treatment for patients with advanced non-small-cell lung cancer (NSCLC) lacking targetable mutations.^{1,2} However, chemotherapy has provided only modest benefit, with limited tolerability. In phase 3 clinical trials, median progression-free survival (PFS) with platinum-based chemotherapy was 4 to 6 months and median overall survival (OS) was 10 to 13 months.³⁻⁸

In two phase 3 studies, nivolumab, a programmed death 1 (PD-1) immune-checkpoint-inhibitor antibody, significantly improved OS compared with docetaxel in patients with metastatic NSCLC who experienced disease progression during or after platinum-based chemotherapy.⁹⁻¹¹ Benefit was seen regardless of PD-1 ligand 1 (PD-L1) expression but was enhanced in nonsquamous NSCLC with increasing PD-L1 expression.^{9,10}

In a multicohort phase 1 study in previously untreated patients with NSCLC (CheckMate 012),¹² preliminary data in the nivolumab monotherapy cohort (n=20) showed durable responses and a favorable safety profile. Among 10 patients with ≥5% PD-L1 expression, the objective response rate (ORR) was 50%, the PFS rate at 24 weeks was 70%, and median PFS was 10.6 months.¹³ Although increasing PD-L1 expression was associated with greater benefit in the expanded cohort, clinical activity was also seen in patients with low or no PD-L1 expression.¹² Owing to the complexity of the immune system, biomarkers for response to immuno-oncology agents beyond PD-L1 expression levels are being explored. Early data support the hypothesis that high tumor mutation burden (TMB) can increase the likelihood of benefit from immunotherapy, as high TMB can enhance immunogenicity by increasing the number of neo-antigens, which are recognized by T cells as non-self, leading to an antitumor immune response.¹⁴

A randomized, open-label, international, phase 3 study that compared the efficacy and safety of nivolumab and investigator's choice of platinum-based chemotherapy as first-line therapy in patients with stage IV or recurrent NSCLC with ≥1% or ≥5% PD-L1 expression was performed. Furthermore, an exploratory analysis was conducted to assess the effects of TMB on treatment outcomes.

### Methods

### Patients

Eligible adult patients had histologically confirmed squamous or nonsquamous stage IV/recurrent NSCLC, ECOG PS 0-1, and measurable disease per RECIST 1.1,¹⁵ and had received no prior systemic anticancer therapy as primary therapy for advanced or metastatic disease. Patients with central nervous system metastases were eligible if adequately treated and neurologically returned to baseline ≥2 weeks before randomization. Eligible patients had to be off corticosteroids or on a stable or decreasing dose of ≤10 mg daily prednisone (or equivalent). Prior palliative radiotherapy, if completed ≥2 weeks before randomization, and prior adjuvant or neoadjuvant chemotherapy ≥6 months before enrollment were permitted. Patients with an autoimmune disease or known EGFR mutations or ALK translocations sensitive to available targeted therapy were excluded. Only patients with ≥1% PD-L1 expression were randomized.

### PD-L1 Analysis for Patient Selection

Fresh or archival tumor-biopsy specimen collected within 6 months before enrollment were tested for PD-L1 by a centralized laboratory using the 28-8 antibody.^{9,10}

### Study Design and Treatment

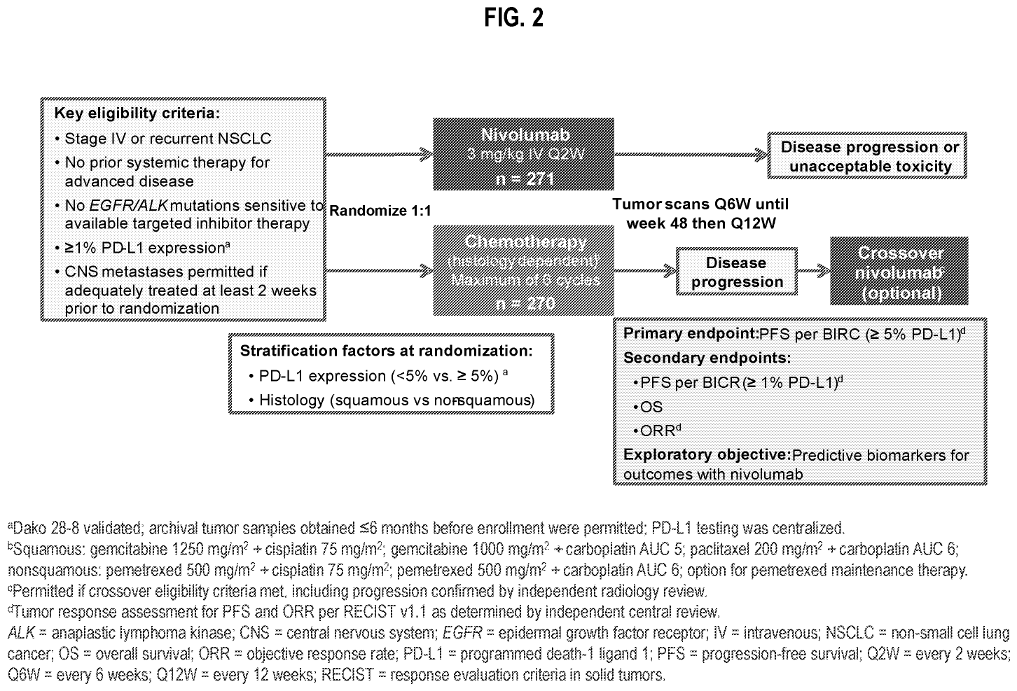

Eligible patients were randomized (1:1) to receive nivolumab 3 mg/kg every 2 weeks or investigator's choice of platinum doublet chemotherapy every 3 weeks for 4 to 6 cycles (FIG. 2). Chemotherapy was continued until disease progression, unacceptable toxicity, or completion of permitted cycles. Maintenance pemetrexed was allowed in patients with nonsquamous NSCLC who had stable disease or response after cycle 4. Treatment with nivolumab beyond progression was permitted if protocol-defined criteria were met. Concomitant systemic corticosteroid treatment (<3-week courses) was allowed for non-autoimmune conditions, including but not limited to treatment-related adverse events (AEs) with a potential immunologic cause.

Randomization was stratified by PD-L1 expression (<5% vs. ≥5%) and tumor histology (squamous vs. nonsquamous). Patients randomized to chemotherapy with progression per RECIST 1.1, assessed by the investigator and confirmed by an independent radiologist, could crossover to nivolumab, provided eligibility criteria were met. For chemotherapy, dose delays and ≤2 dose reductions for toxicity were allowed. For nivolumab, dose delays for toxicity were allowed, but dose reductions were not allowed.

### Endpoints and Assessments

The primary endpoint was PFS based on assessment by blinded independent central review (BICR) in patients with ≥5% PD-L1 expression. Secondary endpoints included PFS per BICR among all randomized patients (≥1% PD-L1 expression), OS among patients with ≥5% PD-L1 expression and among all randomized patients, and ORR per BICR among patients with ≥5% PD-L1 expression.

Tumor response was assessed every 6 weeks until week 48 and every 12 weeks thereafter. Safety assessments included the recording of AEs, graded according to the National Cancer Institute Common Terminology Criteria for Adverse Events, version 4.0.

### Exploratory Biomarker Analysis of TMB

TMB, the total number of somatic missense mutations, was determined in patients with tumor and blood samples sufficient for whole exome sequencing.

DNA and RNA were co-isolated from archival tumor tissue using the Allprep DNA/RNA FFPE kit (Qiagen, Hilden, Germany). DNA from whole blood (germline DNA) was isolated using the QIAamp DNA Blood Midi Kit (Qiagen, Hilden, Germany) following the manufacturer's instructions.

Isolated DNA and RNA was subjected to whole exome capture and sequencing. Genomic DNA (150 ng) was used for library preparation using the Agilent SureSelectXT reagent kit (Agilent Technologies, Santa Clara, USA) with the on-bead modifications of Fisher et al, 2011. (Fisher S, Barry A, Abreu J, et al. A scalable, fully automated process for construction of sequence-ready human exome targeted capture libraries. Genome Biol. 2011; 12 (1): R1). A total of 500 ng of enriched library was used in the hybridization and captured with the SureSelect All Exon v5 (Agilent Technologies, Santa Clara, USA) bait. Following hybridization, the captured libraries were purified according to the manufacturer's recommendations and amplified by polymerase chain reaction (11 cycles). Normalized libraries were pooled and sequenced on the Illumina HiSeq 2500 using 2×100-bp paired-end reads; 45 million reads (100 times the approximate mean target coverage) were sequenced per sample.

Tumor mutation burden determination was performed as follows. Whole exome sequencing data were used to generate tumor mutation burden (total number of missense mutations) for each patient. Missense mutations were identified from paired tumor-germline whole exome sequencing data using two mutation callers. (Weber J A et al. (2016) Sentieon DNA pipeline for variant detection-Software-only solution, over 20× faster than GATK 3.3 with identical results. PeerJ PrePrints 4: e1672v2; Saunders C T et al., Strelka: accurate somatic small-variant calling from sequenced tumor-normal sample pairs. Bioinformatics (2012) 28:1811-7.) The union of the two callers was used to calculate the tumor mutation burden.

For efficacy analyses, patients were grouped according to TMB tertile distribution. Tertile boundaries were 0 to <100, 100 to 242, and ≥243 mutations for low, medium, and high TMB, respectively.

### Study Oversight

The study was designed and data were analyzed jointly by the sponsor (Bristol-Myers Squibb) and a steering committee (D.P.C., M.A.S., L.P.A., and M.R.), with the participation of individual authors. All investigators collected data. The study protocol was approved by the institutional review board or independent ethics committee at each center. The study was conducted in accordance with the International Conference on Harmonisation Guidelines on Good Clinical Practice and the Declaration of Helsinki. An independent data and safety monitoring committee provided oversight of safety and efficacy. This report is based on the final data analysis (Aug. 2, 2016 database lock).

### Statistical Considerations

Sample size estimation for the primary efficacy analysis population (patients with ≥5% PD-L1 expression) was based on an expected median PFS of 7 months in the chemotherapy group and an overall HR of 0.71 favoring nivolumab. A sample size of ˜415 patients was estimated to provide 80% power to detect a difference in treatment effect on the primary endpoint using a log-rank test with a two-sided significance level of 5% after a minimum follow-up of ˜18 months in patients with no disease progression or death.

Comparison of PFS and OS between treatment groups was performed by two-sided log-rank tests stratified by PD-L1 expression level (≥5% vs. <5%; for endpoints in all randomized patients) and tumor histology. A stratified Cox proportional-hazards model including the randomized treatment arm as a single covariate was used to estimate HRs and their associated 95% CIs. The Kaplan-Meier method was used to estimate survival curves. ORRs were compared between treatment arms with a two-sided, stratified Cochran-Mantel-Haenszel test. The Clopper-Pearson method was used to estimate ORRs and their exact 95% CIs.

### Results

### Patients and Treatment

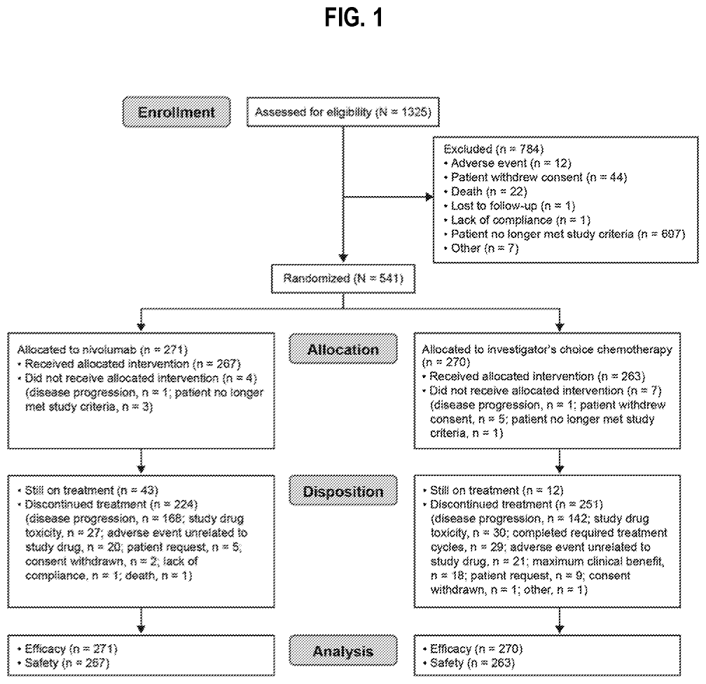

Of 1325 patients enrolled in the study, 541 (40.8%) were randomized, 271 to receive nivolumab and 270 to receive chemotherapy; 784 (59%) patients were not randomized due to non-evaluable PD-L1 samples (6%), PD-L1<1% (23%), or failure to meet other study criteria (30%). During screening, 746 of 1047 (71.3%) patients with evaluable PD-L1 results had PD-L1 expression ≥1%. Overall, 530 patients (98.0% of all randomized patients) received treatment (FIG. 1 and Table 18). The primary efficacy analysis population (patients with ≥5% PD-L1 expression) constituted 78.2% of all randomized patients. Median time from diagnosis to randomization of all patients was 1.9 months (range, 0.3 to 214.9) and 2.0 months (range, 0.5 to 107.3) in the nivolumab and chemotherapy arms, respectively, with 75.6% and 71.9% of patients assigned to the corresponding treatment groups ≤3 months after diagnosis. Overall, 38.6% of patients had prior radiotherapy.

TABLE 18
End-of-Treatment Summary (All Treated Patients).

| | Nivolumab | Chemotherapy |
|---|---|---|
| | n = 267 | n = 263 |
| Patients continuing in the | 43 (16.1) | 12 (4.6) |
| treatment period, n (%) | | |
| Patients not continuing in the | 224 (83.9) | 251 (95.4) |
| treatment period, n (%) | | |
| Reason for not continuing in | | |
| the treatment period, n (%) | | |
| Disease progression | 168 (62.9) | 142 (54.0) |
| Study drug toxicity | 27 (10.1) | 30 (11.4) |
| Death | 1 (0.4) | 0 |
| Adverse event unrelated to study drug | 20 (7.5) | 21 (8.0) |
| Patient request to discontinue study treatment | 5 (1.9) | 9 (3.4) |
| Patient withdrew consent | 2 (0.7) | 1 (0.4) |
| Maximum clinical benefit | 0 | 18 (6.8) |
| Lack of compliance | 1 (0.4) | 0 |
| Other | 0 | 1 (0.4) |
| Completed required treatment cycles | 0 | 29 (11.0) |

Baseline characteristics were generally balanced between the treatment arms except that the chemotherapy arm had higher proportions of female patients (45.2% vs. 32.1%) and patients with ≥50% PD-L1 expression (46.7% vs. 32.5%); whereas the nivolumab arm had a higher proportion of patients with liver metastases (19.9% vs. 13.3%) and greater tumor burden (based on the median sum of target lesion diameters; Table 19).

TABLE 19
Baseline Characteristics of All Randomized Patients.

| | Nivolumab | Chemotherapy | Total |
|---|---|---|---|
| Characteristic | (n = 271) | (n = 270) | (N = 541) |
| Age-yr | | | |
| Median | 63 | 65 | 64 |
| Range | 32-89 | 29-87 | 29-89 |
| Age category-no. (%) | | | |
| <65 years | 148 (54.6) | 133 (49.3) | 281 (51.9) |
| ≥65 to <75 years | 93 (34.3) | 105 (38.9) | 198 (36.6) |
| ≥75 years | 30 (11.1) | 32 (11.9) | 62 (11.5) |
| Sex-no. (%) | | | |
| Male | 184 (67.9) | 148 (54.8) | 332 (61.4) |
| Female | 87 (32.1) | 122 (45.2) | 209 (38.6) |
| Race-no. (%) | | | |
| White | 228 (84.1) | 242 (89.6) | 470 (86.9) |
| Black | 6 (2.2) | 10 (3.7) | 16 (3.0) |
| Asian | 30 (11.1) | 17 (6.3) | 47 (8.7) |
| American Indian or | 1 (0.4) | 0 | 1 (0.2) |
| Alaska native | | | |
| Other | 6 (2.2) | 1 (0.4) | 7 (1.3) |
| Disease stage-no. (%) | | | |
| Stage IV | 255 (94.1) | 244 (90.4) | 499 (92.2) |
| Recurrent | 16 (5.9) | 25 (9.3) | 41 (7.6) |
| Not reported | 0 | 1 (0.4) | 1 (0.2) |
| ECOG performance- | | | |
| status score-no. (%) | | | |
| 0 | 85 (31.4) | 93 (34.4) | 178 (32.9) |
| 1 | 183 (67.5) | 174 (64.4) | 357 (66.0) |
| ≥2 | 2 (0.7) | 3 (1.1) | 5 (0.9) |
| Not reported | 1 (0.4) | 0 | 1 (0.2) |
| Smoking status-no. (%) | | | |
| Never smoker | 30 (11.1) | 29 (10.7) | 59 (10.9) |
| Former smoker | 186 (68.6) | 182 (67.4) | 368 (68.0) |
| Current smoker | 52 (19.2) | 55 (20.4) | 107 (19.8) |
| Unknown | 3 (1.1) | 4 (1.5) | 7 (1.3) |
| Prior systemic | | | |
| therapy-no. (%) | | | |
| Adjuvant | 22 (8.1) | 25 (9.3) | 47 (8.7) |
| Neoadjuvant | 5 (1.8) | 4 (1.5) | 9 (1.7) |
| Prior radiotherapy-no. (%) | 102 (37.6) | 107 (39.6) | 209 (38.6) |
| Tumor histology-no. (%) | | | |
| Squamous cell carcinoma | 66 (24.4) | 64 (23.7) | 130 (24.0) |
| Nonsquamous cell | 205 (75.6) | 206 (76.3) | 411 (76.0) |
| carcinoma | | | |
| Selected sites of metastatic | | | |
| lesions-no. (%) | | | |
| Brain | 33 (12.2) | 36 (13.3) | 69 (12.8) |
| Liver | 54 (19.9) | 36 (13.3) | 90 (16.6) |
| Median sum of | 82.5 (14-218) | 68.0 (15-272) | 76.0 (14-272) |
| target lesion | | | |
| diameters, mm (range) | | | |
| PD-L1 expression | | | |
| level-no. (%) | | | |
| ≥5% | 208 (76.8) | 210 (77.8) | 418 (77.3) |
| ≥25% | 132 (48.7) | 164 (60.7) | 296 (54.7) |
| ≥50% | 88 (32.5) | 126 (46.7) | 214 (39.6) |
| ≥75% | 56 (20.7) | 74 (27.4) | 130 (24.0) |

ECOG denotes Eastern Cooperative Oncology Group.

Minimum follow-up for OS was 13.7 months. Median duration of therapy was 3.7 months (range, 0.0 to 26.9+) for nivolumab and 3.4 months (range, 0.0 to 20.9+) for chemotherapy (regimens shown in Table 20); 38.0% of treated patients received maintenance pemetrexed. A total of 77 (28.8%) randomized patients treated with nivolumab received nivolumab beyond investigator-assessed RECIST 1.1 progression; 26 received >6 nivolumab doses beyond progression.

TABLE 20
Chemotherapy Study Treatments
(All Treated Patients).

| | Chemotherapy |
|---|---|
| Study treatments, n (%) | n = 263 |
| Pemetrexed/carboplatin | 115 (43.7) |
| Pemetrexed/cisplatin | 86 (32.7) |
| Gemcitabine/carboplatin | 33 (12.5) |
| Gemcitabine/cisplatin | 13 (4.9) |
| Paclitaxel/carboplatin | 16 (6.1) |
| Maintenance pemetrexed, n (%) | 100 (38.0) |

Among patients with ≥5% PD-L1 expression in the nivolumab arm, 43.6% received subsequent systemic cancer therapy, and 18.7% of treated patients remained on nivolumab at the time of database lock. In the chemotherapy arm, 64.2% of patients received subsequent systemic therapy, including 60.4% who received nivolumab as crossover treatment within the study (57.5%) and/or in clinical practice after the study (3.3%) (Table 21).

TABLE 21
Subsequent Systemic Therapy in Patients
with ≥5% PD-L1 Expression.

| | Nivolumab | Chemotherapy |
|---|---|---|
| | n = 211 | n = 212 |
| Subsequent systemic therapy, n (%) | 92 (43.6) | 136 (64.2) |
| Immunotherapy, n (%) | 3 (1.4) | 128 (60.4) |
| Crossover nivolumab | 0 | 122 (57.5) |
| Post-study nivolumab | 2 (0.9) | 7 (3.3) |
| Ipilimumab | 1 (0.5) | 0 |
| ALK/EGFR tyrosine kinase inhibitors, | 12 (5.7) | 6 (2.8) |
| n (%) | | |
| Experimental therapy, n (%) | 2 (0.9) | 2 (0.9) |
| Chemotherapy and other systemic | 88 (41.7) | 30 (14.2) |
| anticancer agents, n (%) | | |

### Efficacy

### Primary Efficacy Analysis Population and all Randomized Patients

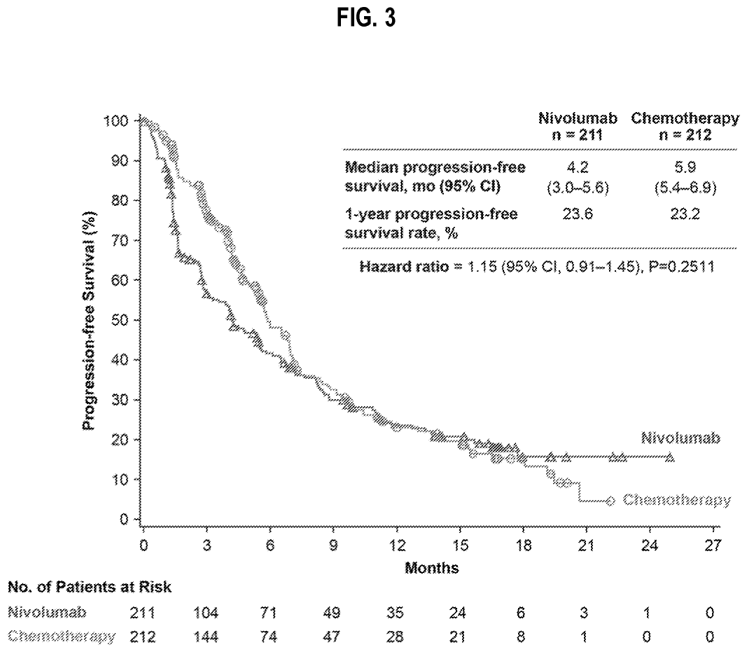

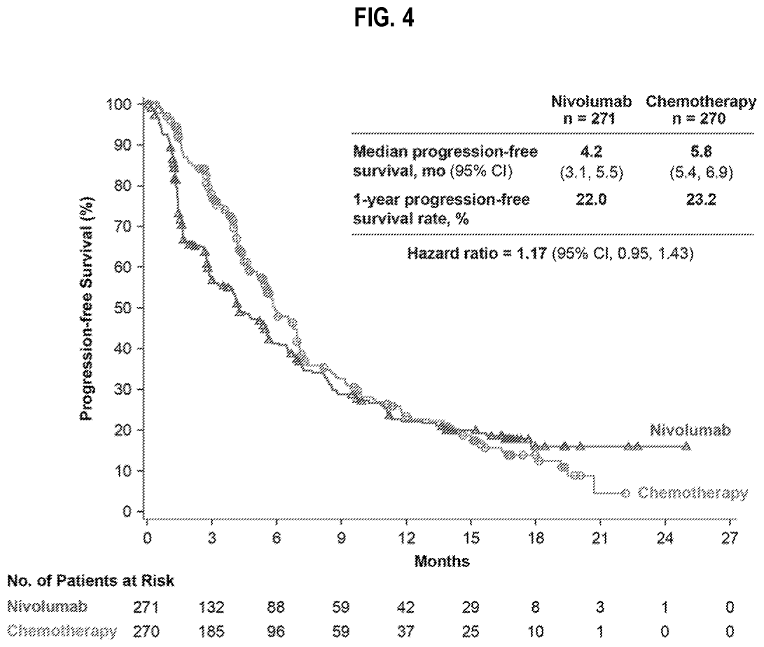

In the primary efficacy analysis population (≥5% PD-L1 expression), there was no significant difference in PFS between treatment arms (FIG. 3). Median PFS was 4.2 months (95% CI, 3.0 to 5.6) with nivolumab and 5.9 months (95% CI, 5.4 to 6.9) with chemotherapy (HR, 1.15; 95% CI, 0.91 to 1.45; P=0.2511). Similar results were obtained for all randomized patients (FIG. 4).

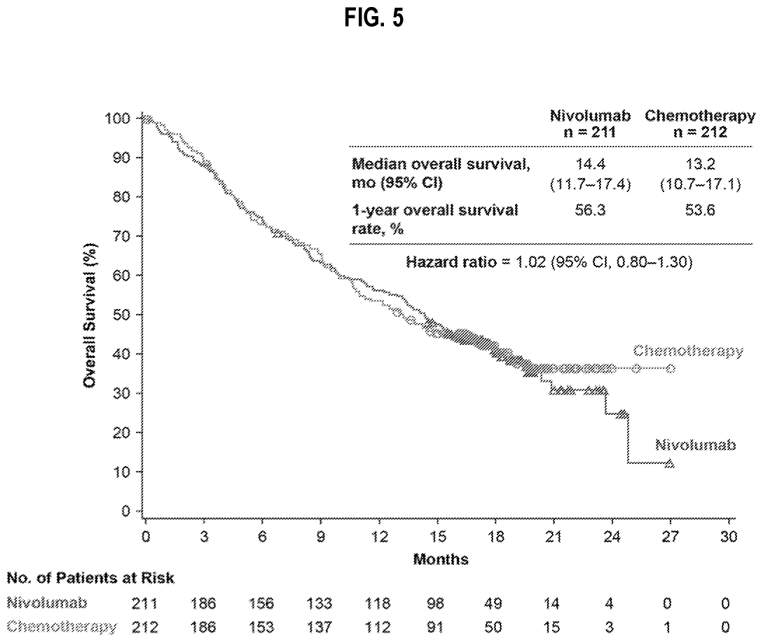

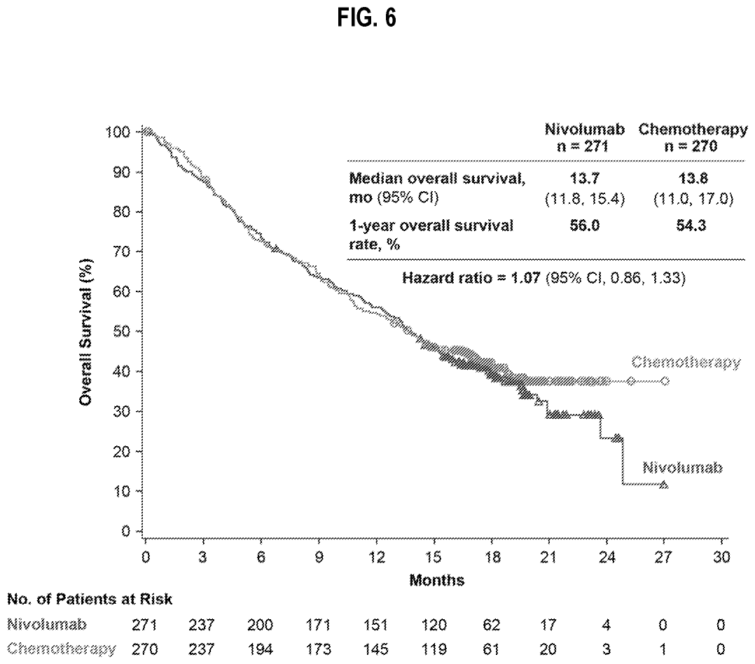

Median OS in the primary efficacy analysis population was 14.4 months (95% CI, 11.7 to 17.4) with nivolumab and 13.2 months (95% CI, 10.7 to 17.1) with chemotherapy (HR, 1.02; 95% CI, 0.80 to 1.30) (FIG. 5). Similar results were obtained for all randomized patients (FIG. 6).

The ORR among patients with ≥5% PD-L1 expression was 26.1% with nivolumab and 33.5% with chemotherapy; the difference was not statistically significant (Table 22). Compared with the nivolumab arm, the chemotherapy arm had a lower proportion of patients with a best response of progressive disease (9.9% vs. 27.5%). Median time to response was similar in the two treatment arms, whereas median duration of response was more than twice as long with nivolumab as with chemotherapy (12.1 vs. 5.7 months; Table 22).

TABLE 22
Tumor Response with Nivolumab versus
Chemotherapy in Patients with ≥5% PD-L1
Expression.*

| | Nivolumab | Chemotherapy |
|---|---|---|
| Variable | (n = 211) | (n = 212) |
| Objective response† | | |
| No. of patients | 55 | 71 |
| % of patients (95% CI) | 26.1 (20.3-32.5) | 33.5 (27.2-40.3) |
| Estimated odds ratio (95% CI) | 0.70 (0.46-1.06) | |
| P value | 0.0957 | |
| Best overall response-no. (%) | | |
| Complete response | 4 (1.9) | 1 (0.5) |
| Partial response | 51 (24.2) | 70 (33.0) |
| Stable disease | 81 (38.4) | 100 (47.2) |
| Progressive disease | 58 (27.5) | 21 (9.9) |
| Could not be determined | 17 (8.1) | 20 (9.4) |
| Time to response-mo‡§ | | |
| Median | 2.8 | 2.6 |
| Range | 1.2-13.2 | 1.2-9.8 |
| Duration of response-mo‡¶ | | |
| Median | 12.1 | 5.7 |
| Range | 1.7-19.4+ | 1.4-21.0+ |

*Data are based on an Aug. 2, 2016, database lock. PD-L1 denotes programmed death-ligand 1.
†Objective response was assessed according to the Response Evaluation Criteria in Solid Tumors, version 1.1 by independent central review. The 95% confidence interval (CI) is based on the Clopper-Pearson method. The analysis was stratified by tumor histology. The strata-adjusted odds ratio and the two-sided P value were calculated with the use of the Cochran-Mantel-Haenszel method.
‡The analysis was performed with data from all the patients who had a response (55 patients in the nivolumab group and 71 in the investigator's choice chemotherapy group).
§The time to response was defined as the time from randomization to the date of first documented complete or partial response.
¶Results were calculated with the use of the Kaplan-Meier method. The duration of response was defined as the time between the date of first response and the date of first documented event of progression, death, or last tumor assessment that was evaluated before subsequent therapy (data-censoring date).

### Selected Subgroups

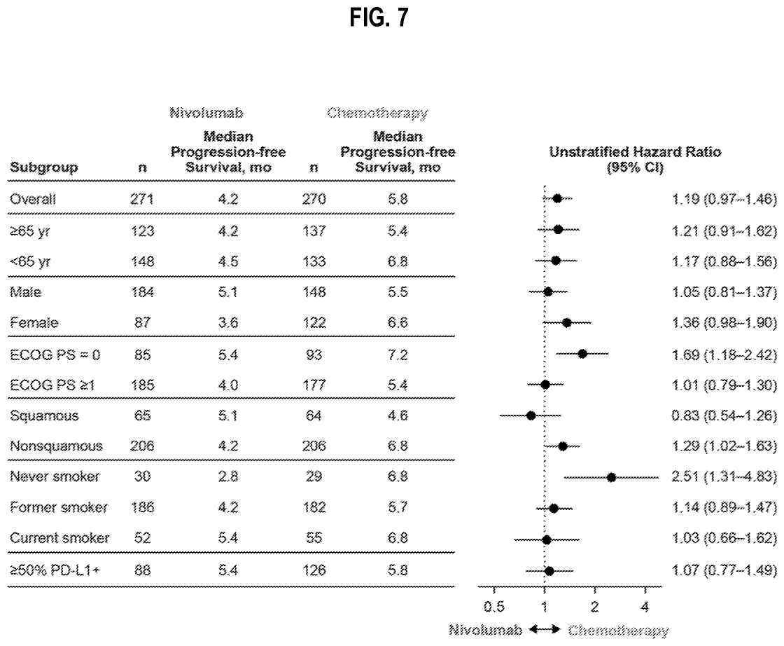

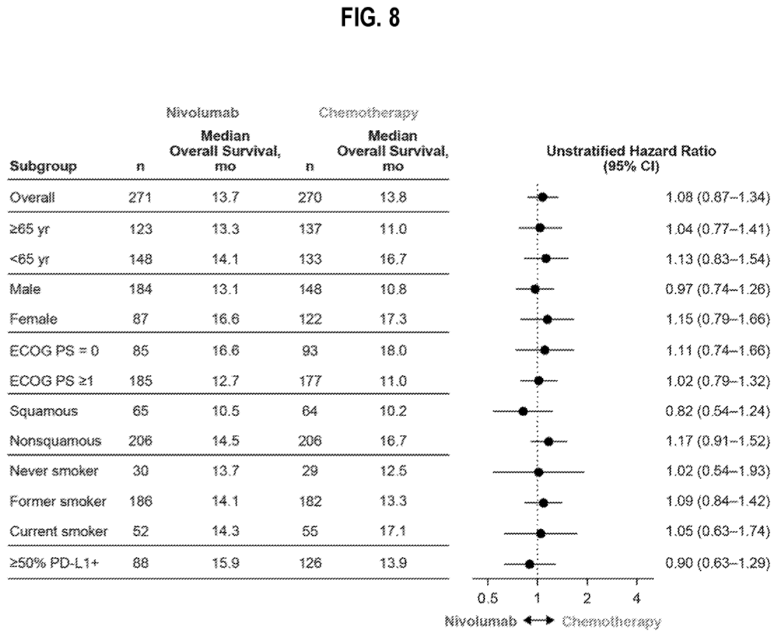

Across most predefined subgroups, PFS and OS were consistent with the overall study results (FIGS. 7-8). The only predefined stratified subgroup was histology; patients with squamous histology had numerically improved PFS and OS with nivolumab versus chemotherapy (FIGS. 7-8). In the exploratory subgroup analysis of patients with ≥50% PD-L1 expression, the HRs for PFS and OS were 1.07 (95% CI, 0.77 to 1.49) and 0.90 (95% CI, 0.63 to 1.29), respectively. The ORR was 34.1% (95% CI, 24.3% to 45.0%) for nivolumab and 38.9% (95% CI, 30.3% to 48.0%) for chemotherapy. As this subgroup was not stratified, the nivolumab arm had fewer patients than the chemotherapy arm (88 vs. 126), and the imbalance in sex noted in the overall population was even more pronounced in this subgroup (25.0% vs. 43.7% female).

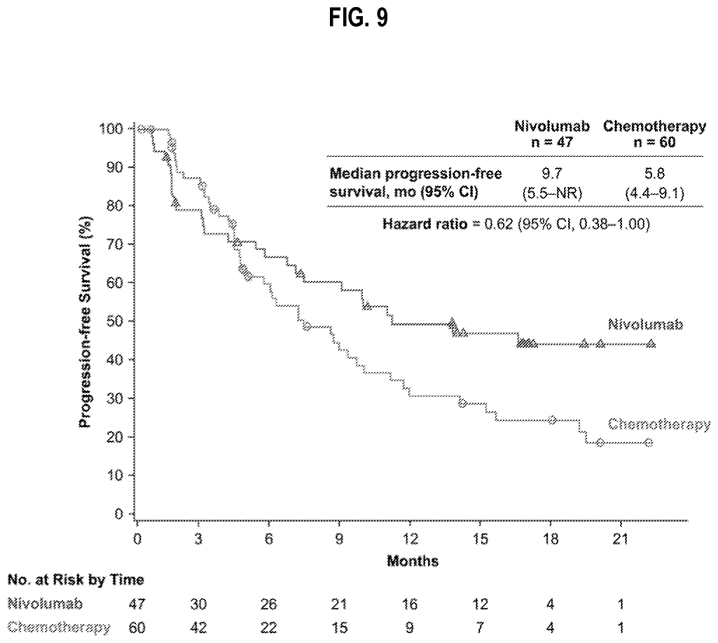

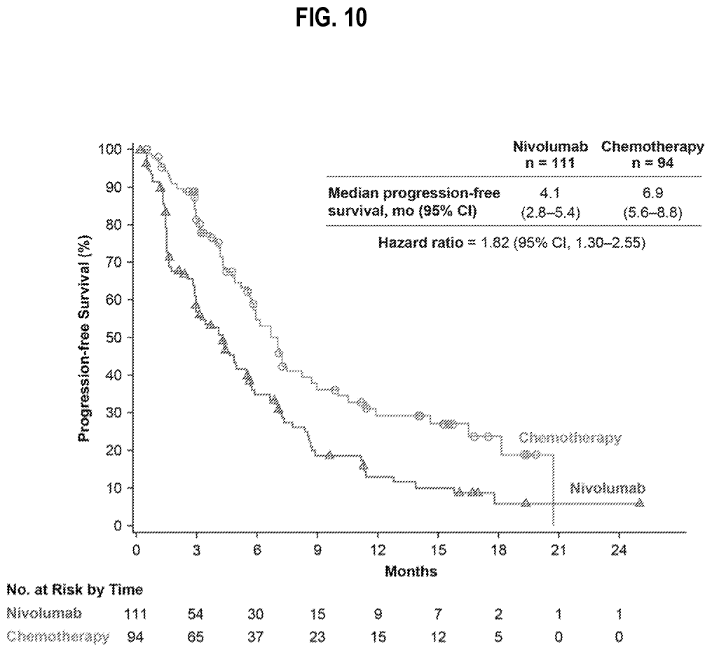

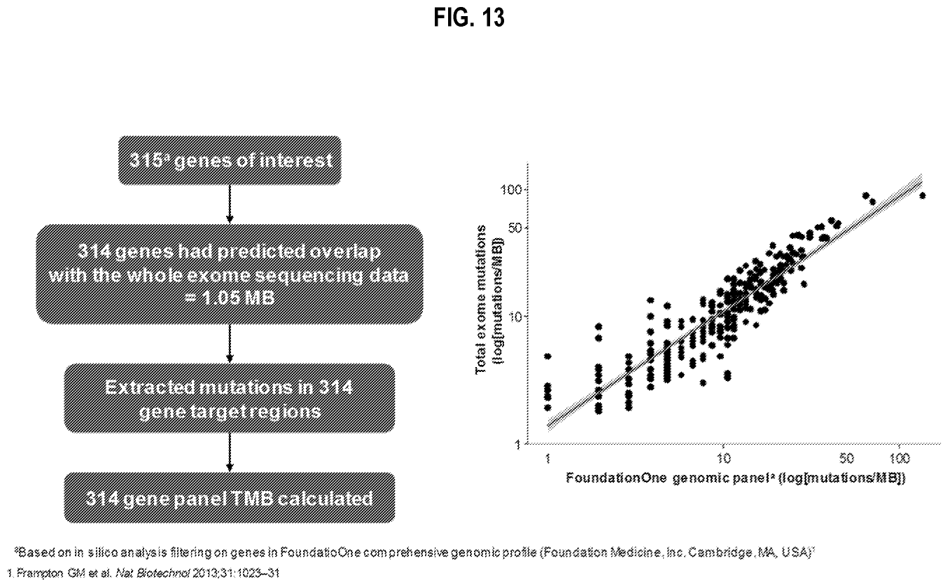

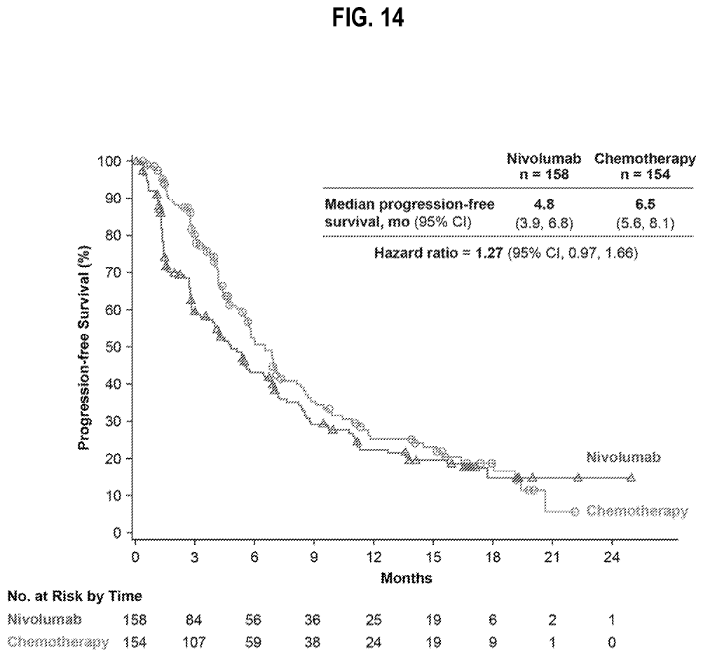

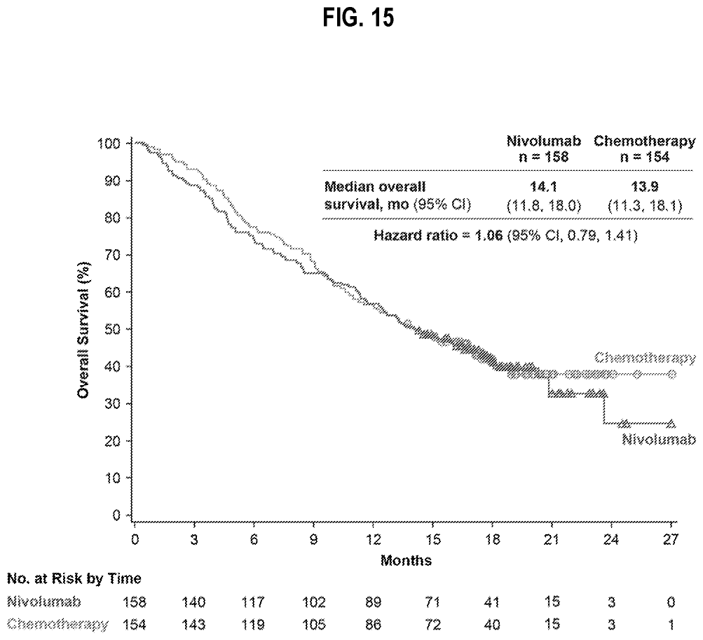

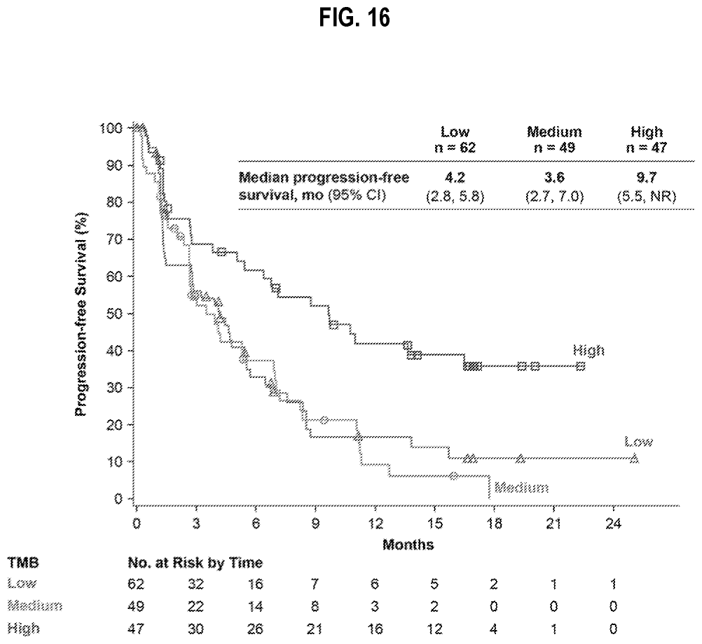

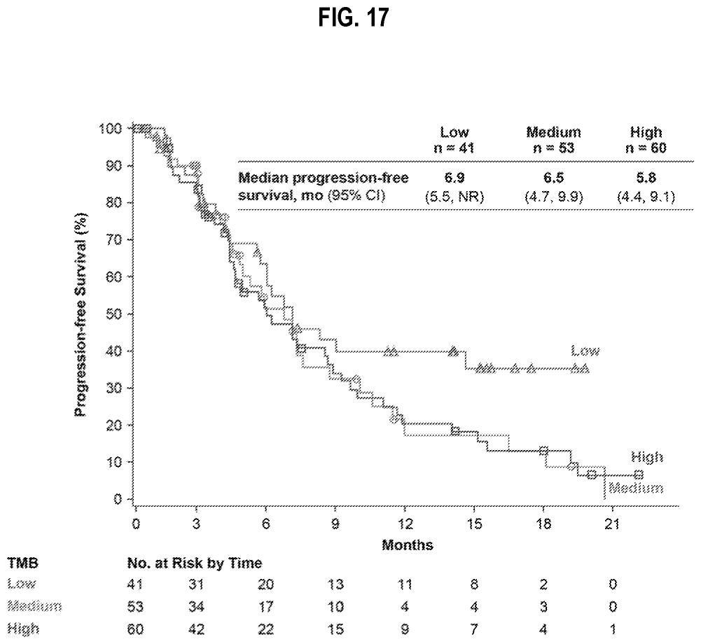

An exploratory analysis was conducted in 312 patients (57.7% of randomized patients to assess the impact of TMB on outcomes (Tables 23-25; FIGS. 9-17)). The percentage of patients with high TMB (upper tertile, 33%) was imbalanced between treatment arms (nivolumab: 29.7% vs. chemotherapy: 39.0%, Table 25). Baseline characteristics, PFS, and OS (Tables 24-25 and FIGS. 14-15) were generally consistent with all randomized patients.

TABLE 23
Sample Attrition During Tumor Mutation Burden Determination.

| | Tumor | Germline |
|---|---|---|
| Patients, n (%) | DNA | DNA^{a} |
| Randomized | 541 (100) | 541 (100) |
| Samples available for DNA extraction^{b} | 485 (90) | 452 (84) |
| DNA available for sequencing | 408 (75) | 452 (84) |
| Successful preparation of next-generation | 402 (74) | 452 (84) |
| sequencing library | | |
| Passed internal quality control^{c} | 320 (59) | 432 (80) |
| Matched tumor-germline exome sequences | 312 (58) | |
| for TMB analysis^{d} | | |

^{a}Matched germline DNA from whole blood was used to distinguish germline single-nucleotide polymorphisms from somatic missense mutations in the tumor DNA
^{b}Samples were not available for various reasons, including but not limited to lack of patient pharmacogenetic consent, samples exhausted for PD-L1 testing, or poor tissue sampling
^{c}Internal quality control included evaluation of factors including but not limited to discordance between tumor and germline DNA, too few sequence reads, and too many repetitive artifact sequence reads
^{d}Eight patients with available tumor DNA sequences did not have matched germline DNA sequences

TABLE 24
Baseline Characteristics of All Randomized
Patients and Patients with Evaluable
Tumor Mutation Data.

| | All | Patients with |
|---|---|---|
| | randomized | evaluable |
| | patients | TMB data |
| Characteristic | (n = 541) | (n = 312) |
| Age, year | | |
| Median | 64 | 65 |
| Range | 29-89 | 32-89 |
| Sex, n (%) | | |
| Male | 332 (61.4) | 187 (59.9) |
| Female | 209 (38.6) | 125 (40.1) |
| Disease stage, n (%) | | |
| Stage IV | 499 (92.2) | 291 (93.3) |
| Recurrent | 41 (7.6) | 20 (6.4) |
| Not reported | 0 | 1 (0.3) |
| ECOG performance-status | | |
| score, n (%) | | |
| 0 | 178 (32.9) | 100 (32.1) |
| 1 | 357 (66.0) | 208 (66.7) |
| ≥2 | 5 (0.9) | 3 (1.0) |
| Not reported | 1 (0.2) | 1 (0.3) |
| Smoking status, n (%) | | |
| Never smoker | 59 (10.9) | 29 (9.3) |
| Former smoker | 368 (68.0) | 223 (71.5) |
| Current smoker | 107 (19.8) | 56 (17.9) |
| Unknown | 7 (1.3) | 4 (1.3) |
| Tumor histology, n (%) | | |
| Squamous cell carcinoma | 130 (24.0) | 71 (22.8) |
| Nonsquamous cell | 411 (76.0) | 241 (77.2) |
| carcinoma | | |
| PD-L1 expression level, n (%) | | |
| ≥5% | 418 (77.3) | 252 (80.8) |
| ≥25% | 296 (54.7) | 185 (59.3) |
| ≥50% | 214 (39.6) | 130 (41.7) |
| Tumor mutation burden, n (%) | | |

ECOG = Eastern Cooperative Oncology Group.

TABLE 25
Baseline Characteristics of Patients with
Evaluable Tumor Mutation Data by
Treatment Arm.

| | Nivolumab | Chemotherapy |
|---|---|---|
| Characteristic | (n = 158) | (n = 154) |
| Age, year | | |
| Median | 65 | 64 |
| Range | 32-89 | 34-87 |
| Age category, n (%) | | |
| <65 years | 76 (48.1) | 78 (50.6) |
| ≥65 to <75 years | 59 (37.3) | 57 (37.0) |
| ≥75 years | 23 (14.6) | 19 (12.3) |
| Sex, n (%) | | |
| Male | 105 (66.5) | 82 (53.2) |
| Female | 53 (33.5) | 72 (46.8) |
| Race, n (%) | | |
| White | 126 (79.7) | 135 (87.7) |
| Black | 4 (2.5) | 6 (3.9) |
| Asian | 22 (13.9) | 12 (7.8) |
| American Indian or Alaska native | 1 (0.6) | 0 |
| Other | 5 (3.2) | 1 (0.6) |
| Disease stage, n (%) | | |
| Stage IV | 150 (94.9) | 141 (91.6) |
| Recurrent | 8 (5.1) | 12 (7.8) |
| Not reported | 0 | 1 (0.6) |
| ECOG performance-status score, n (%) | | |
| 0 | 46 (29.1) | 54 (35.1) |
| 1 | 110 (69.6) | 98 (63.6) |
| ≥2 | 1 (0.6) | 2 (1.3) |
| Not reported | 1 (0.6) | 0 |
| Smoking status, n (%) | | |
| Never smoker | 16 (10.1) | 13 (8.4) |
| Former smoker | 116 (73.4) | 107 (69.5) |
| Current smoker | 24 (15.2) | 32 (20.8) |
| Unknown | 2 (1.3) | 2 (1.3) |
| Prior systemic therapy, n (%) | | |
| Adjuvant | 13 (8.2) | 12 (7.8) |
| Neoadjuvant | 2 (1.3) | 2 (1.3) |
| Prior radiotherapy, n (%) | 51 (32.3) | 60 (39.0) |
| Tumor histology, n (%) | | |
| Squamous cell carcinoma | 36 (22.8) | 35 (22.7) |
| Nonsquamous cell carcinoma | 122 (77.2) | 119 (77.3) |
| Selected sites of metastatic lesions, n (%) | | |
| Brain | 18 (11.4) | 21 (13.6) |
| Liver | 34 (21.5) | 31 (20.1) |
| Median sum of target lesion diameters, | 79.5 (14-218) | 70 (15-272) |
| mm (range) | | |
| PD-L1 expression level, n (%) | | |
| ≥5% | 125 (79.1) | 127 (82.5) |
| ≥25% | 86 (54.4) | 99 (64.3) |
| ≥50% | 57 (36.1) | 73 (47.4) |
| Tumor mutation burden, n (%) | | |
| Low (<33 percentile) | 62 (39.2) | 41 (26.6) |
| Medium (33-66 percentile) | 49 (31.0) | 53 (34.4) |
| High (>66 percentile) | 47 (29.7) | 60 (39.0) |

ECOG = Eastern Cooperative Oncology Group.

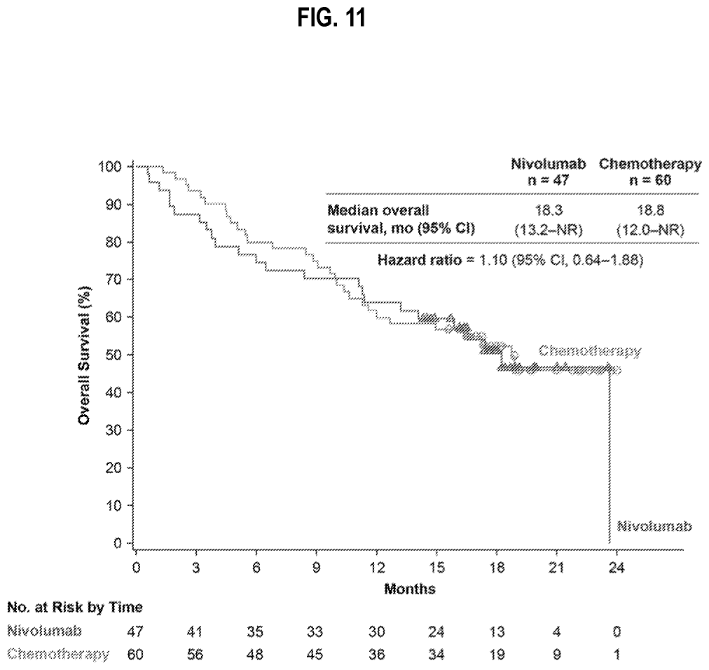

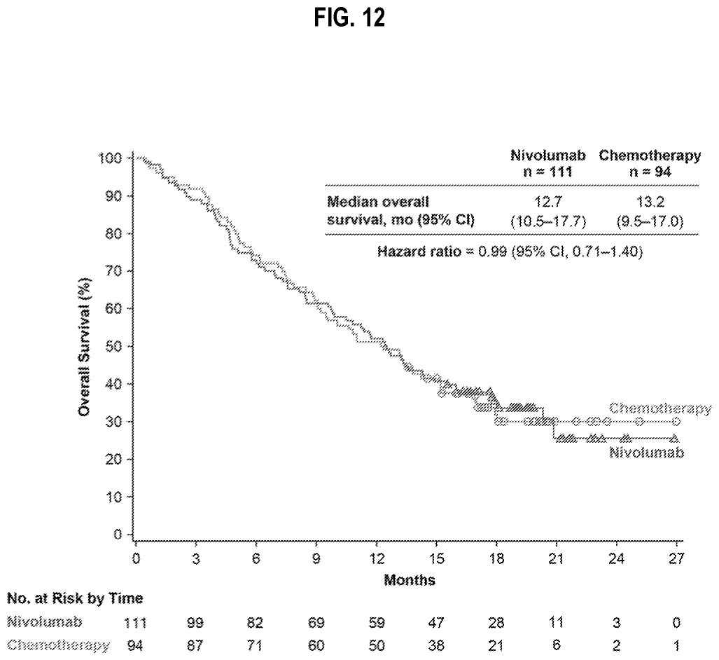

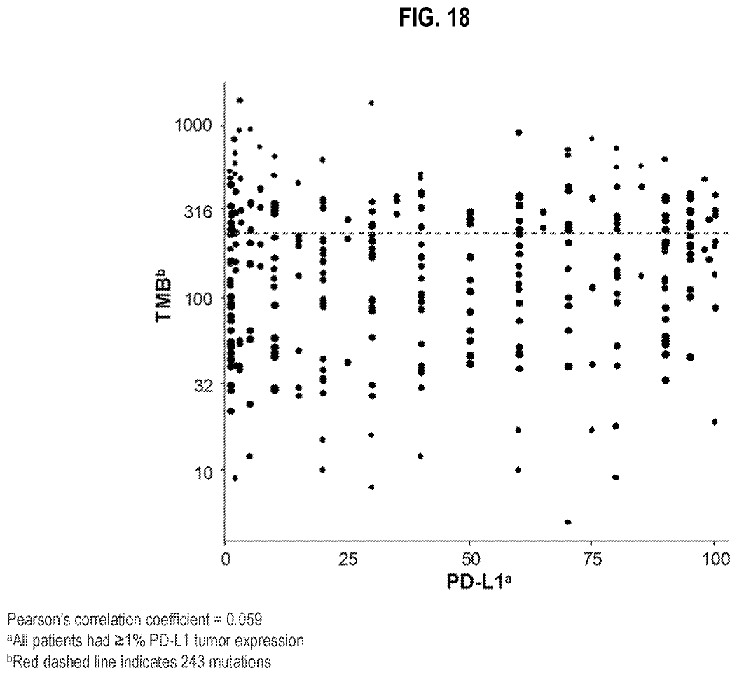

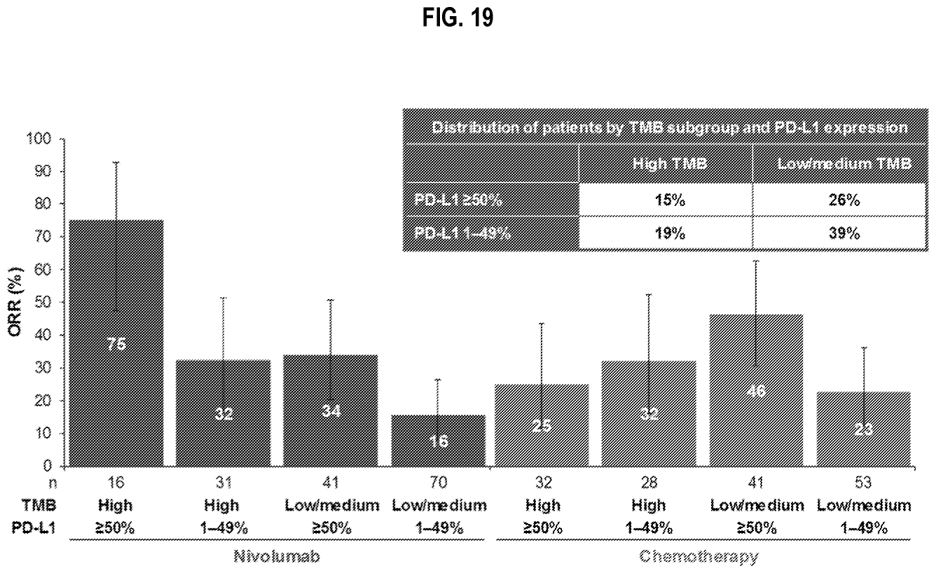

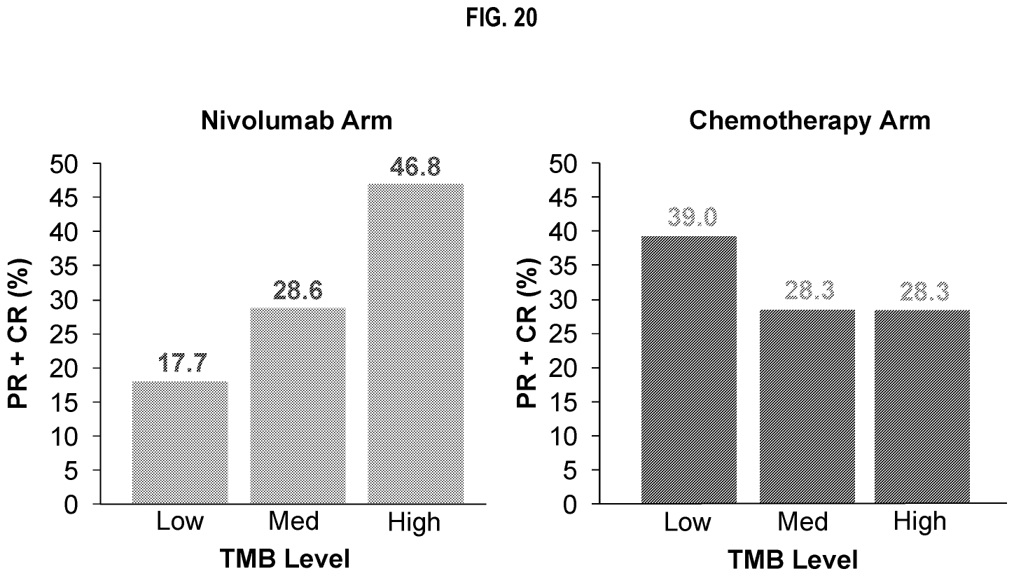

In patients with high TMB, ORR was higher in the nivolumab arm (46.8%) than in the chemotherapy arm (28.3%) (Table 26). PFS was improved with nivolumab versus chemotherapy (median, 9.7 vs. 5.8 months) in patients with high TMB (HR, 0.62; 95% CI, 0.38 to 1.00; FIG. 9). OS was similar between arms regardless of TMB (FIGS. 11-12); however, 65% of patients with high TMB in the chemotherapy arm received nivolumab after crossover. There was no significant association between TMB and PD-L1 expression (Pearson's correlation coefficient=0.059; FIG. 18).

TABLE 26
Response by Tumor Mutation Burden in Evaluable Patients.

| | Tumor mutation burden | | | |
|---|---|---|---|---|
| | | | Low/medium | |
| | Low | Medium | (pooled)* | High |
| Nivolumab | n = 62 | n = 49 | n = 111 | n = 47 |
| Complete or partial | 11 (17.7) | 14 (28.6) | 25 (22.5) | 22 (46.8) |
| response, n (%) | | | | |
| Stable disease, n (%) | 25 (40.3) | 20 (40.8) | 45 (40.5) | 15 (31.9) |
| Progressive disease, n | 21 (33.9) | 11 (22.4) | 32 (28.9) | 7 (14.9) |
| (%) | | | | |
| Could not be | 5 (8.1) | 4 (8.2) | 9 (8.1) | 3 (6.4) |
| determined, n (%) | | | | |
| Chemotherapy | n = 41 | n = 53 | n = 104 | n = 60 |
| Complete or partial | 16 (39.0) | 15 (28.3) | 31 (29.8) | 17 (28.3) |
| response, n (%) | | | | |
| Stable disease, n (%) | 19 (46.3) | 30 (56.6) | 49 (47.1) | 32 (53.3) |
| Progressive disease, n | 1 (2.4) | 3 (5.7) | 4 (3.8) | 7 (11.7) |
| (%) | | | | |
| Could not be | 5 (12.2) | 5 (9.4) | 10 (9.6) | 4 (6.7) |
| determined, n (%) | | | | |

*Data for patients with low and medium tumor mutation burden were pooled, because median PFS was similar for low and medium tumor mutation burden in either treatment arm.

### Safety

Treatment-related AEs of any grade occurred in 71.2% and 92.4% of patients treated with nivolumab and chemotherapy, respectively; the proportion of patients with treatment-related grade 3/4 AEs was lower with nivolumab (17.6%) than chemotherapy (50.6%) (Tables 11-12). Rates of treatment-related serious AEs were similar with nivolumab and chemotherapy; however, treatment-related AEs leading to discontinuation of study drug were less common with nivolumab than chemotherapy (9.7% vs. 13.3%; Table 27 and Tables 29-31).

TABLE 27
Treatment-Related Adverse Events Reported
in at Least 10% of Patients Treated with
Nivolumab or Chemotherapy.*

| | Nivolumab | | Chemotherapy | |
|---|---|---|---|---|
| | (n = 267) | | (n = 263) | |
| | Any Grade | Grade 3 or 4 | Any Grade | Grade 3 or 4 |
| Event | number of patients with an event (percent) | | | |
| Any event | 190 (71.2) | 47 (17.6) | 243 (92.4) | 133 (50.6) |
| Any serious event | 46 (17.2) | 35 (13.1) | 48 (18.3) | 41 (15.6) |
| Any event leading to | 26 (9.7) | 21 (7.9) | 35 (13.3) | 17 (6.5) |
| discontinuation | | | | |
| Fatigue | 56 (21.0) | 3 (1.1) | 93 (35.4) | 14 (5.3) |
| Diarrhea | 37 (13.9) | 3 (1.1) | 34 (12.9) | 5 (1.9) |
| Decreased appetite | 32 (12.0) | 1 (0.4) | 73 (27.8) | 4 (1.5) |
| Nausea | 31 (11.6) | 1 (0.4) | 127 (48.3) | 5 (1.9) |
| Rash | 26 (9.7) | 2 (0.7) | 15 (5.7) | 1 (0.4) |
| Vomiting | 15 (5.6) | 0 | 60 (22.8) | 5 (1.9) |
| Constipation | 9 (3.4) | 0 | 29 (11.0) | 0 |
| Anemia | 9 (3.4) | 1 (0.4) | 113 (43.0) | 46 (17.5) |
| Asthenia | 8 (3.0) | 0 | 28 (10.6) | 4 (1.5) |
| Thrombocytopenia | 2 (0.7) | 1 (0.4) | 38 (14.4) | 22 (8.4) |
| Platelet count | 2 (0.7) | 0 | 33 (12.5) | 9 (3.4) |
| decreased | | | | |
| Neutrophil count | 1 (0.4) | 1 (0.4) | 36 (13.7) | 20 (7.6) |
| decreased | | | | |
| Neutropenia | 0 | 0 | 48 (18.3) | 29 (11.0) |

*Data are based on an Aug. 2, 2016, database lock. Safety analyses included all the patients who received at least one dose of study drug. Included are events reported from the time of the first dose of study drug to 30 days after the last dose or to the time of the first dose of nivolumab crossover, whichever came first.

TABLE 28
Treatment-Related Adverse Events in ≥5%
of Patients Treated with Nivolumab or
Chemotherapy.

| | Nivolumab | | Chemotherapy | |
|---|---|---|---|---|
| | n = 267 | | n = 263 | |
| Event, n (%) | Any Grade | Grade 3-4 | Any Grade | Grade 3-4 |
| Any event | 190 (71.2) | 47 (17.6) | 243 (92.4) | 133 (50.6) |
| Fatigue | 56 (21.0) | 3 (1.1) | 93 (35.4) | 14 (5.3) |
| Diarrhea | 37 (13.9) | 3 (1.1) | 34 (12.9) | 5 (1.9) |
| Decreased appetite | 32 (12.0) | 1 (0.4) | 73 (27.8) | 4 (1.5) |
| Nausea | 31 (11.6) | 1 (0.4) | 127 (48.3) | 5 (1.9) |
| Rash | 26 (9.7) | 2 (0.7) | 15 (5.7) | 1 (0.4) |
| Aspartate | 23 (8.6) | 7 (2.6) | 12 (4.6) | 1 (0.4) |
| aminotransferase | | | | |
| increased | | | | |
| Pruritus | 22 (8.2) | 0 | 7 (2.7) | 1 (0.4) |
| Alanine | 19 (7.1) | 7 (2.6) | 14 (5.3) | 2 (0.8) |
| aminotransferase | | | | |
| increased | | | | |
| Hypothyroidism | 17 (6.4) | 0 | 1 (0.4) | 0 |
| Vomiting | 15 (5.6) | 0 | 60 (22.8) | 5 (1.9) |
| Pyrexia | 14 (5.2) | 0 | 13 (4.9) | 1 (0.4) |
| Rash maculopapular | 14 (5.2) | 1 (0.4) | 4 (1.5) | 0 |
| Constipation | 9 (3.4) | 0 | 29 (11.0) | 0 |
| Anemia | 9 (3.4) | 1 (0.4) | 113 (43.0) | 46 (17.5) |
| Asthenia | 8 (3.0) | 0 | 28 (10.6) | 4 (1.5) |
| Dysgeusia | 7 (2.6) | 0 | 21 (8.0) | 0 |
| Peripheral edema | 6 (2.2) | 0 | 22 (8.4) | 0 |
| Blood creatinine | 5 (1.9) | 1 (0.4) | 16 (6.1) | 0 |
| increased | | | | |
| Stomatitis | 5 (1.9) | 0 | 15 (5.7) | 1 (0.4) |
| Hypomagnesemia | 4 (1.5) | 0 | 25 (9.1) | 2 (0.8) |
| Mucosal | 4 (1.5) | 0 | 20 (7.6) | 0 |
| inflammation | | | | |
| Alopecia | 3 (1.1) | 0 | 23 (8.7) | 0 |
| Thrombocytopenia | 2 (0.7) | 1 (0.4) | 38 (14.4) | 22 (8.4) |
| Platelet count | 2 (0.7) | 0 | 33 (12.5) | 9 (3.4) |
| decreased | | | | |
| White blood cell | 2 (0.7) | 0 | 26 (9.9) | 9 (3.4) |
| count decreased | | | | |
| Neutrophil count | 1 (0.4) | 1 (0.4) | 36 (13.7) | 20 (7.6) |
| decreased | | | | |
| Peripheral sensory | 1 (0.4) | 0 | 15 (5.7) | 0 |
| neuropathy | | | | |
| Neutropenia | 0 | 0 | 48 (18.3) | 29 (11.0) |
| Leukopenia | 0 | 0 | 16 (6.1) | 9 (3.4) |

TABLE 29
Treatment-Related Serious Adverse Events
in ≥2% of Patients Treated with
Nivolumab or Chemotherapy.

| | Nivolumab | | Chemotherapy | |
|---|---|---|---|---|
| | n = 267 | | n = 263 | |
| | Any | Grade | Any | Grade |
| Event, n (%) | Grade | 3-4 | Grade | 3-4 |
| Any event | 46 (17.2) | 35 (13.1) | 48 (18.3) | 41 (15.6) |
| Pneumonitis | 7 (2.6) | 4 (1.5) | 0 | 0 |
| Aspartate | 6 (2.2) | 6 (2.2) | 0 | 0 |
| aminotransferase | | | | |
| increased | | | | |
| Anemia | 0 | 0 | 13 (4.9) | 10 (3.8) |
| Febrile neutropenia | 0 | 0 | 6 (2.3) | 6 (2.3) |
| Thrombocytopenia | 0 | 0 | 6 (2.3) | 6 (2.3) |

TABLE 30
Treatment-Related Adverse Events
Leading to Discontinuation of Nivolumab.

| | Nivolumab | |
|---|---|---|
| | n = 267 | |
| | Any | Grade |
| Event, n (%) | Grade | 3-4 |
| Any event | 26 (9.7) | 21 (7.9) |
| Aspartate aminotransferase increased | 5 (1.9) | 5 (1.9) |
| Alanine aminotransferase increased | 5 (1.9) | 5 (1.9) |
| Pneumonitis | 3 (1.1) | 3 (1.1) |
| Colitis | 2 (0.7) | 2 (0.7) |
| Transaminases increased | 1 (0.4) | 1 (0.4) |
| Interstitial lung disease | 1 (0.4) | 1 (0.4) |
| Autoimmune colitis | 1 (0.4) | 0 |
| Diarrhea | 1 (0.4) | 0 |
| Gastritis | 1 (0.4) | 0 |
| Nausea | 1 (0.4) | 1 (0.4) |
| Rash | 1 (0.4) | 1 (0.4) |
| Rash maculopapular | 1 (0.4) | 1 (0.4) |
| Rash papular | 1 (0.4) | 1 (0.4) |
| Stevens-Johnson syndrome | 1 (0.4) | 1 (0.4) |
| Malaise | 1 (0.4) | 0 |
| Multiple organ dysfunction | 1 (0.4) | 1 (0.4) |
| Adrenal insufficiency | 1 (0.4) | 1 (0.4) |
| Cholestasis | 1 (0.4) | 1 (0.4) |
| Hypersensitivity | 1 (0.4) | 1 (0.4) |
| Arthritis | 1 (0.4) | 0 |
| Pericardial effusion malignant | 1 (0.4) | 1 (0.4) |
| Aphasia | 1 (0.4) | 1 (0.4) |
| Confused state | 1 (0.4) | 1 (0.4) |

TABLE 31
Treatment-Related Adverse Events Leading
to Discontinuation of Chemotherapy

| | Chemotherapy | |
|---|---|---|
| | n = 263 | |
| | Any | Grade |
| Event, n (%) | Grade | 3-4 |
| Any event | 35 (13.3) | 17 (6.5) |
| Anemia | 5 (1.9) | 3 (1.1) |
| Blood creatinine increased | 5 (1.9) | 0 |
| Febrile neutropenia | 4 (1.5) | 4 (1.5) |
| Neutropenia | 3 (1.1) | 1 (0.4) |
| Fatigue | 3 (1.1) | 2 (0.8) |
| General physical health deterioration | 2 (0.8) | 2 (0.8) |
| Decreased appetite | 2 (0.8) | 1 (0.4) |
| Asthenia | 2 (0.8) | 0 |
| Chronic kidney disease | 2 (0.8) | 0 |
| Renal infarction | 1 (0.4) | 1 (0.4) |
| Renal failure | 1 (0.4) | 0 |
| Renal function test abnormal | 1 (0.4) | 0 |
| Thrombocytopenia | 1 (0.4) | 1 (0.4) |
| Myocardial infarction | 1 (0.4) | 1 (0.4) |
| Pneumonia | 1 (0.4) | 1 (0.4) |
| Erysipelas | 1 (0.4) | 1 (0.4) |
| Sepsis | 1 (0.4) | 1 (0.4) |
| Bronchospasm | 1 (0.4) | 1 (0.4) |
| Pneumonitis | 1 (0.4) | 0 |
| Gastrointestinal hemorrhage | 1 (0.4) | 1 (0.4) |
| Nausea | 1 (0.4) | 0 |
| Vomiting | 1 (0.4) | 0 |
| Neurotoxicity | 1 (0.4) | 0 |
| Peripheral sensory neuropathy | 1 (0.4) | 0 |
| Tinnitus | 1 (0.4) | 0 |
| Peripheral edema | 1 (0.4) | 0 |

The most common treatment-related select AEs (those with a potential immunologic cause) were skin-related events in the nivolumab arm and gastrointestinal events in the chemotherapy arm (Table 32).

TABLE 32
Treatment-Related Select Adverse Events^{a}
in Patients Treated with Nivolumab or
Chemotherapy.

| | Nivolumab | | Chemotherapy | |
|---|---|---|---|---|
| Select Adverse Event | n = 267 | | n = 263 | |
| | Any | Grade | Any | Grade |
| Category, n (%) | Grade | 3-4 | Grade | 3-4 |
| Skin | 63 (23.6) | 5 (1.9) | 25 (9.5) | 1 (0.4) |
| Gastrointestinal | 39 (14.6) | 6 (2.2) | 34 (12.9) | 5 (1.9) |
| Hepatic | 33 (12.4) | 9 (3.4) | 26 (9.9) | 2 (0.8) |
| Pulmonary | 14 (5.2) | 6 (2.2) | 1 (0.4) | 0 |
| Hypersensitivity/infusion | 11 (4.1) | 1 (0.4) | 3 (1.1) | 1 (0.4) |
| reaction | | | | |
| Renal | 5 (1.9) | 1 (0.4) | 18 (6.8) | 0 |

^{a}Select adverse events are those with potential immunologic etiology that require frequent monitoring/intervention; includes events reported from the time of the first dose of study drug to 30 days after the last dose or to the time of the first dose of nivolumab crossover, whichever came first.

Five deaths were attributed to study treatment, including two deaths in the nivolumab arm (one each from multi-organ failure and pneumonitis) and three deaths in the chemotherapy arm (one from sepsis and two from febrile neutropenia).

### DISCUSSION

This study did not meet the primary endpoint of superior PFS for first-line nivolumab monotherapy versus chemotherapy in patients with stage IV/recurrent NSCLC and ≥5% PD-L1 expression. OS was similar in the two treatment arms, comparing favorably with historical controls of first-line platinum-based chemotherapy.³⁻⁸ Given that nivolumab therapy prolongs survival of previously treated patients with advanced NSCLC,^{9,10} the high frequency of subsequent nivolumab treatment can have contributed to the favorable OS in the chemotherapy arm. Imbalances in baseline characteristics can have favored the chemotherapy arm, including better prognostic disease characteristics (i.e., fewer liver metastases, lower tumor burden, and a higher proportion of females).^{3,4,16}

Analyses comparing treatment efficacy in patients with ≥50% PD-L1 expression were not prespecified in this study, and the two arms had a major imbalance in the number of patients (88 vs. 126), thereby limiting conclusions that can be drawn in this subgroup. In contrast, the KEYNOTE-024 trial assessed the activity of pembrolizumab versus chemotherapy only in patients with ≥50% PD-L1-expressing chemotherapy-naive advanced NSCLC.¹⁷ Other differences between the studies have been outlined in a recent review article,¹⁸ but examples include the different assays to assess PD-L1 tumor expression, criteria related to prior radiotherapy and on-study corticosteroid use, and imbalances in patient characteristics between treatment arms (e.g., sex in the study and lower percentage of never-smokers in the immunotherapy arm of KEYNOTE-024 [3.2%] vs. chemotherapy).^{17,18}

KEYNOTE-024 established a role for pembrolizumab as first-line treatment in patients with NSCLC with ≥50% PD-L1 expression (median PFS, 10.3 months; ORR, 45%); however, an unmet need remains for the majority of patients in this setting, and biomarkers in addition to PD-L1 continue to be examined due to the complexity of tumor-immune interactions to better predict outcomes with immuno-oncology therapy.

In an exploratory analysis, among patients evaluable for TMB, nivolumab improved ORR and PFS vs. chemotherapy in the high TMB subgroup (nivolumab ORR, 46.8%; median PFS, 9.7 months). There was not an OS difference between treatment arms in the high TMB subgroup, which can be explained in part by high crossover (65%) to nivolumab in the chemotherapy arm. Nevertheless, the high TMB subgroup had notable OS (>18 months median OS). TMB level and tumor PD-L1 expression did not appear to be associated and patients with both high TMB and ≥50% PD-L1 expression can have a greater likelihood of response to nivolumab than those with only one or neither of these factors. Taken together, the findings of this exploratory analysis support the hypothesis that immunotherapy has enhanced activity in patients with high TMB¹⁴ and warrant prospective confirmation.

In the broad PD-L1-expressing population in this study, nivolumab monotherapy was comparable to platinum-based chemotherapy and provides an encouraging foundation for future first-line combination strategies, which can improve long-term outcomes and expand the patient population to benefit from anti-PD-1 therapy. Combining nivolumab with ipilimumab, 19.20 can which depletes regulatory T cells involved in the suppression of host immune response,^{19,20} improve antitumor activity.²¹ Findings from CheckMate 012 suggest that this combination can enhance clinical activity in the first-line NSCLC setting. In patients with ≥1% PD-L1 expression, ORR was doubled in the nivolumab plus ipilimumab cohorts compared with the nivolumab monotherapy cohort (57% vs. 28%), and the one-year OS rate was 87%.^{12,22,23} A phase 3 study (CheckMate 227; NCT02477826) is evaluating the efficacy and safety of nivolumab plus ipilimumab or chemotherapy in chemotherapy-naïve patients with stage IV/recurrent NSCLC. Furthermore, several ongoing phase 3 studies are evaluating dual checkpoint-inhibitor blockade or PD-1 inhibitors plus chemotherapy in NSCLC (e.g., NCT02453282, NCT02367781, and NCT02578680).

In conclusion, nivolumab monotherapy did not improve PFS compared with platinum-based chemotherapy as first-line treatment for stage IV/recurrent NSCLC in a broad population of patients with ≥5% PD-L1 expression. OS with single-agent nivolumab was robust and comparable to platinum doublet chemotherapy. Moreover, this is the first phase 3 trial with an exploratory endpoint to evaluate whether PD-1 inhibitor therapy has enhanced benefit by improving outcomes in patients with high TMB. Nivolumab had an improved safety profile compared with chemotherapy, and no new safety signals were observed.

### Example 2

### Examination of a Targeted Gene Panel (FOUNDATIONONE®) Versus Whole Exome Sequencing to Evaluate Concordance Using Samples from a Phase 3 Study of First-Line Nivolumab in Stage IV or Recurrent Non-Small-Cell Lung Cancer

TMB is defined as the number of somatic mutations per megabase of tumor genome examined. It was hypothesized that one can calculate TMB by sequencing fewer genes compared to whole exome sequencing. Sequencing using FOUNDATIONONE® has previously been validated using 249 cancer specimens. See, e.g., Frampton G M et al. Nat Biotechnol. 2013; 31:1023-1031.

To assess whether TMB values are equivalent and whether concordance exists between whole exome sequencing (WES)-derived and FOUNDATIONONE® assay data, TMB data from patients enrolled in the study (from Example 1) were generated using the two sequencing platforms: WES and FOUNDATIONONE®.

### Methods

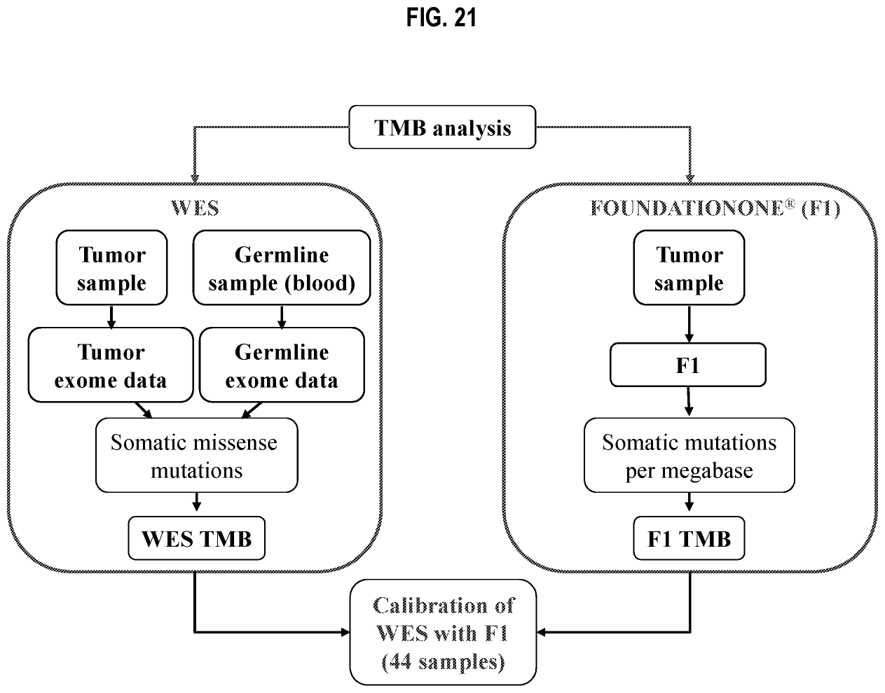

TMB was assessed in the DNA of formalin-fixe, paraffin-embedded (FFPE) tumor samples using 2 hybridization-capture/NGS methods. For WES, the coding regions of 21,522 genes were analyzed. Briefly, tumor exome data and germline (blood) exome data were collected and compared to identify somatic missense mutations (FIG. 21). TMB was then defined as the total number of missense mutations in the tumor exome.

For FOUNDATIONONE®, a targeted gene panel of 315 caner-related genes was analyzed. TMB was defined as the number of somatic mutations per megabase of tumor genome examined. The sensitivity and accuracy of this analysis was previously validated using 249 cancer specimens, and this method has been used to assess TMB across many tumor types (see Frampton et al., Nat. Biotechnol. 31:1023 (2013)), including a recent study of 102,292 tumors (see Chalmers et al., Genome Med. 9:34 (2017)). FIG. 21 illustrates the experimental design.

### Results

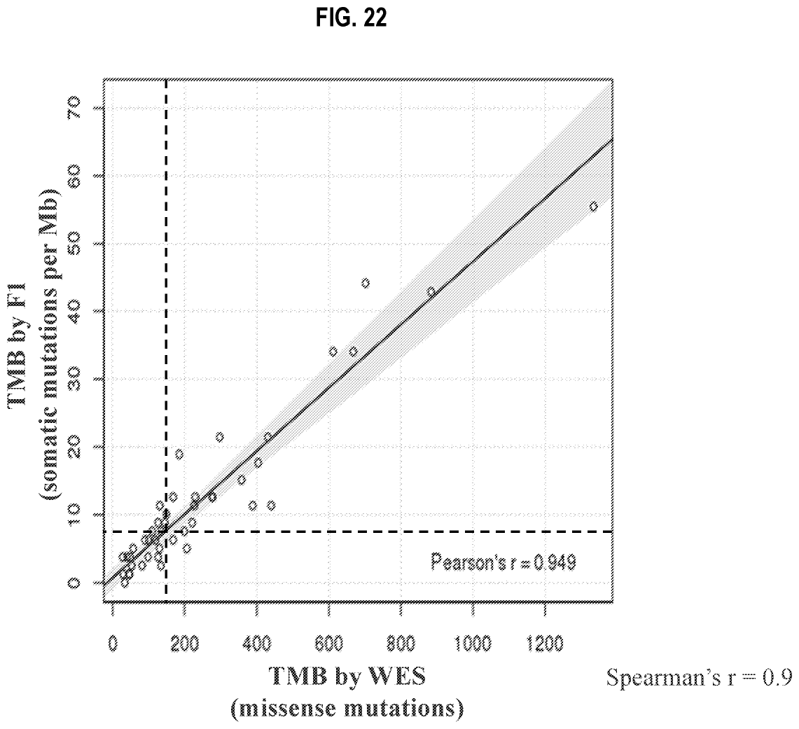

TMB determined by whole exome sequencing (WES) was plotted linearly against TMB determined by FOUNDATIONONE® sequencing (F1). As shown in FIG. 22, TMB is highly correlated between both techniques, and many missense mutations identified via whole exome sequencing and many somatic mutations identified via FOUNDATIONONE® sequencing fall within 0.95-confidence bounds, which was calculated using the bootstrap (quantile) method (Spearman's r=0.9).

In order to determine the TMB concordance between FOUNDATIONONE® and whole exome sequencing, a TMB value of 148 missense mutations was set as the median (FIG. 22, vertical dashed line). At the same data point, it was calculated that there were 7.64 somatic mutations per megabase in the 44 samples using FOUNDATIONONE® sequencing (FIG. 22, horizontal dashed line). As shown in Table 33, the correlation between both sequencing approaches is bridged. Thus, FOUNDATIONONE® sequencing can be used to identify tumor mutation burden in patients with Stage IV or Recurrent Non-Small-Cell Lung Cancer who were enrolled in a Phase 3 Study of First-line Nivolumab.

TABLE 33
Bridging TMB by FOUNDATIONONE ®
sequencing and whole exome sequencing.

| | FoundationOne ® | FoundationOne ® |
|---|---|---|
| | above line | below line |
| Whole exome | 19 | 3 |
| sequencing | | |
| above median | | |
| Whole exome | 3 | 19 |
| sequencing | | |
| below median | | |

Calibration curves were used to project TMB data derived from whole exome sequencing to those based on FOUNDATIONONE® sequencing. Overall, there was 86% agreement (73-94; 95% Wilson confidence interval) between whole exome sequencing and FOUNDATIONONE® sequencing. Regarding positive correlations, there also was 86% agreement (67-95; 95% Wilson confidence interval) between whole exome sequencing and FOUNDATIONONE® sequencing. And regarding negative correlations, there also was 86% agreement (67-95; 95% Wilson confidence interval) between whole exome sequencing and FOUNDATIONONE® sequencing. These data demonstrate that bridging whole exome sequencing and FOUNDATIONONE® sequencing facilitates the transition of whole exome sequencing-derived biomarker data to FOUNDATIONONE® sequencing.

This study ultimately supports that hypothesis that TMB data across testing platforms can be harmonized. Because TMB is an emerging biomarker for precision immuno-oncology therapy, the ability to harmonize data across testing platforms will help provide alternative testing options.

### Example 3

Patients with recurrent small cell lung cancer (SCLC) have limited treatment options and poor survival. Initial results from a clinical trial of patients with SCLC showed durable responses and encouraging survival with nivolumab alone or in combination with ipilimumab. Twenty-six percent of patients receiving a combination of nivolumab and ipilimumab had overall survival rates over 2 years, as compared to 14% of patients receiving nivolumab monotherapy. These data supported inclusion of nivolumab with or without ipilimumab in NCCN guidelines for treatment of SCLC.

Tumor PD-L1 expression is uncommon in SCLC, and responses have been observed regardless of PD-L1 status. Improved biomarkers are needed for immunotherapy in SCLC. Previously, subjects having a high TMB were found to have higher rates of progression free survival (PFS) following treatment with nivolumab monotherapy as compared to subjects having low or medium TMB. SCLC is almost exclusively found in patients with history of smoking and is characterized by high TMB. An association between TMB and efficacy has been seen with nivolumab in NSCLC and bladder cancer, and with ipilimumab in melanoma. High TMB may be associated with enhanced benefit from nivolumab+ ipilimumab in SCLC. The present study explores the use of tumor mutation burden (TMB) as a predictive biomarker for nivolumab with or without ipilimumab in SCLC.

### Study Design

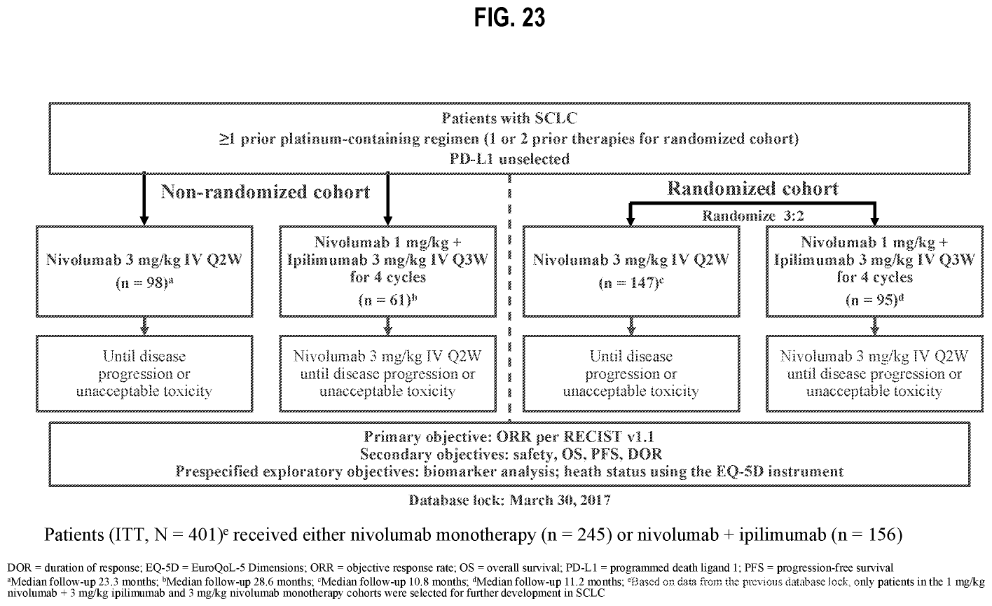

Subjects were selects who had previously been diagnosed with SCLC, and who had previously received at least one prior platinum-containing regimen (FIG. 23). Non-randomized and randomized (3:2) patients received either (1) a nivolumab monotherapy comprising 3 mg/kg nivolumab administered by IV every two weeks until disease progression or unacceptable toxicity; or (2) nivolumab/ipilimumab combination therapy comprising 1 mg/kg nivolumab and 3 mg/kg ipilimumab administered by IV every three weeks for four cycles, followed by nivolumab monotherapy of 3 mg/kg nivolumab administered by IV every two weeks until disease progression or unacceptable toxicity.

The primary objective was to measure the objective response rate (ORR) by per RECIST v1.1. Secondary objectives included monitoring safety, overall survival (OS), progression free survival (PFS), and duration of response (DOR). Prespecified exploratory objectives included biomarker analysis and health status using the EQ-5D instrument.

TMB was determined by whole exome sequencing, using an Illumina HiSeq 2500 using 2×100-bp paired-end reads, and calculated as the total number of nonsynonymous missense mutations in the tumor. For exploratory analyses, patients were divided into 3 subgroups based on TMB tertile.

### Baseline

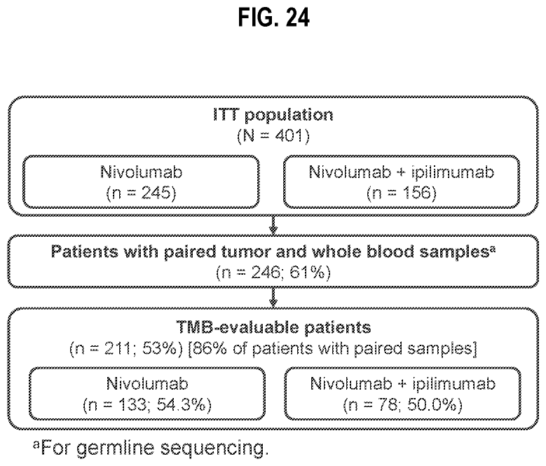

A total of 245 subjects were included (ITT) for nivolumab monotherapy, of which 133 were TMB evaluable (Table 34 and FIG. 24). A total of 156 subjects were included (ITT) for nivolumab/ipilimumab combination therapy, of which 78 were TMB evaluable (Table 34 and FIG. 24).

TABLE 34
Baseline Characteristics

| | | | Nivolumab + | |
|---|---|---|---|---|
| | Nivolumab | | ipilimumab | |
| | | TMB- | | TMB- |
| | ITT | evaluable | ITT | evaluable |
| | (n = 245) | (n = 133) | (n = 156) | (n = 78) |
| Age, median (range), | 63 (29-83) | 63 (29-83) | 65 (37-91) | 65 (37-80) |
| years | | | | |
| Male, n (%) | 60 | 59 | 61 | 67 |
| Smoking status, % | | | | |
| Current/former | 94 | 95 | 94 | 94 |
| smoker | | | | |
| Never smoker | 5 | 5 | 5 | 6 |
| ECOG PS, % | | | | |
| 0 | 30 | 32 | 31 | 30 |
| 1 | 70 | 68 | 68 | 69 |
| Tumor PD-L1 | | | | |
| expression, % | | | | |
| ≥1% | 10 | 13 | 12 | 10 |
| <1% | 61 | 67 | 58 | 65 |
| Unknown | 29 | 20 | 30 | 24 |
| Study cohort, % | | | | |
| Non-randomized | 40 | 38 | 39 | 32 |
| Randomized | 60 | 62 | 61 | 68 |

### Results

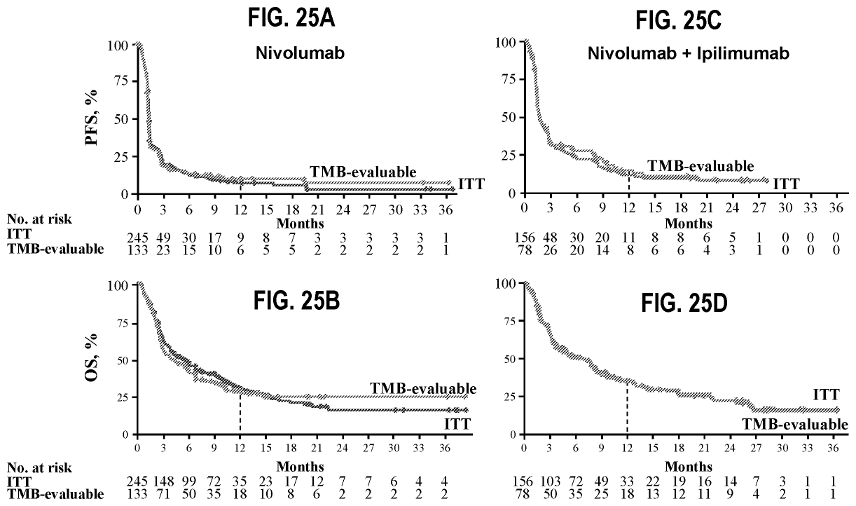

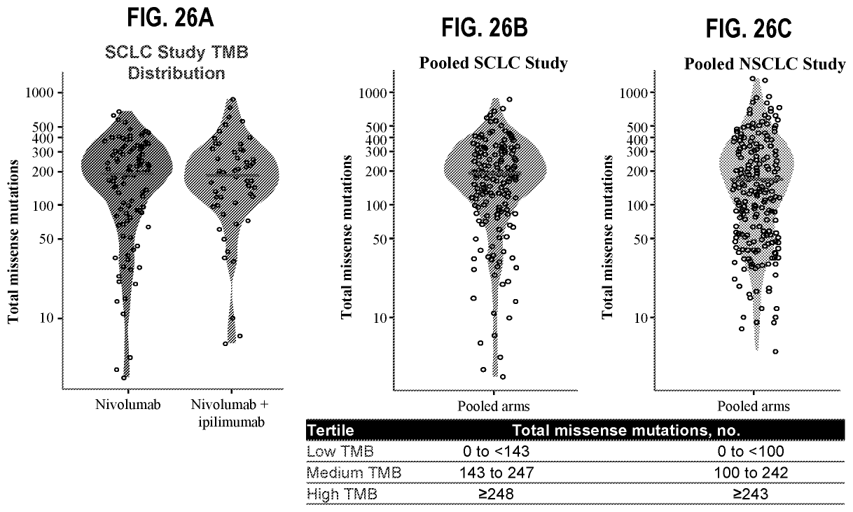

Progression free survival (PFS; FIGS. 25A and 25C) and overall survival (OS; FIGS. 25B and 25D) were comparable between the ITT patients and the subset that was TMB-evaluable for nivolumab monotherapy (FIGS. 25A and 25B) and nivolumab/ipilimumab combination therapy (FIGS. 25C and 25D). ORR in ITT and TMB-evaluable patients, respectively, was 11.4% and 11.3% with nivolumab monotherapy and 21.8% and 28.2% with nivolumab/ipilimumab combination therapy. TMB distribution for patients receiving nivolumab monotherapy or nivolumab/ipilimumab combination therapy are shown in FIG. 26A. When pooled (FIG. 26B), the distribution of the total missense mutations in the SCLC cohort was comparable to the distribution of total missense mutations in a recent non-small cell lung cancer (NSCLC) study (FIG. 26C).

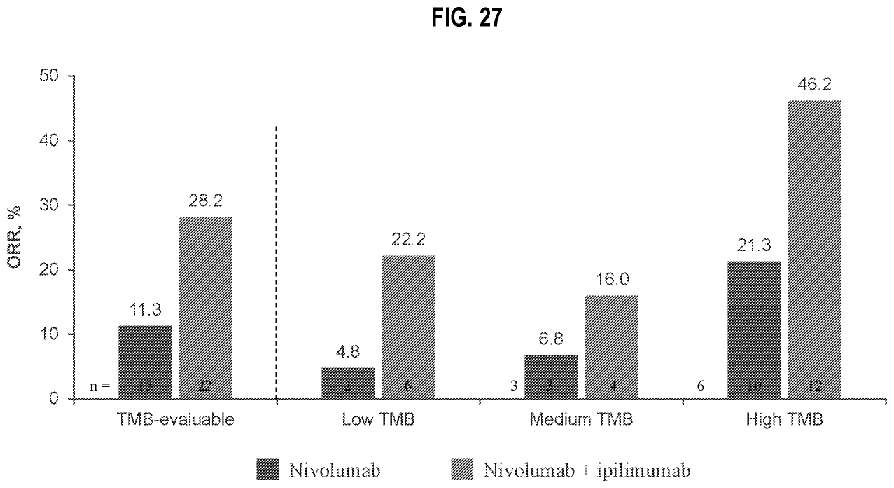

Overall response rate (ORR) was higher in TMB-evaluable subjects administered the nivolumab/ipilimumab combination therapy (28.2%) than in subjects administered nivolumab monotherapy (11.3%) (FIG. 27). When stratified by TMB, the greatest effect was observed for subjects having a high TMB. Subjects with a low TMB treated with nivolumab monotherapy or ipilimumab monotherapy showed ORRs of about 4.8% and 22.2%, respectively. Subjects with a medium TMB treated with nivolumab monotherapy or ipilimumab monotherapy showed ORRs of about 6.8% and 16.0%, respectively. Subjects with a high TMB treated with nivolumab monotherapy or ipilimumab monotherapy showed ORRs of about 21.3% and 46.2%, respectively.

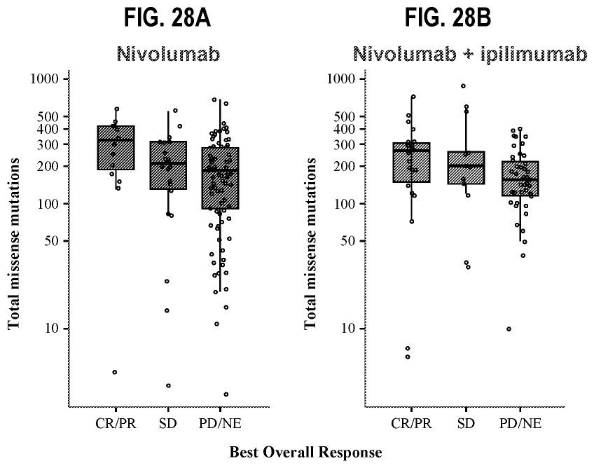

In general, subjects experiencing a better response had a higher number of missense tumor mutations. Subjects administered nivolumab monotherapy experiencing a complete response (CR) or a partial response (PR) had an average of 325 missense mutations, those experiencing stable disease had an average of 211.5 missense mutations, and those experiencing stable disease had an average of 185.5 missense mutations (FIG. 28A). Subjects administered nivolumab/ipilimumab combination therapy experiencing a complete response (CR) or a partial response (PR) had an average of 266 missense mutations, those experiencing stable disease had an average of 202 missense mutations, and those experiencing stable disease had an average of 156 missense mutations (FIG. 28B).

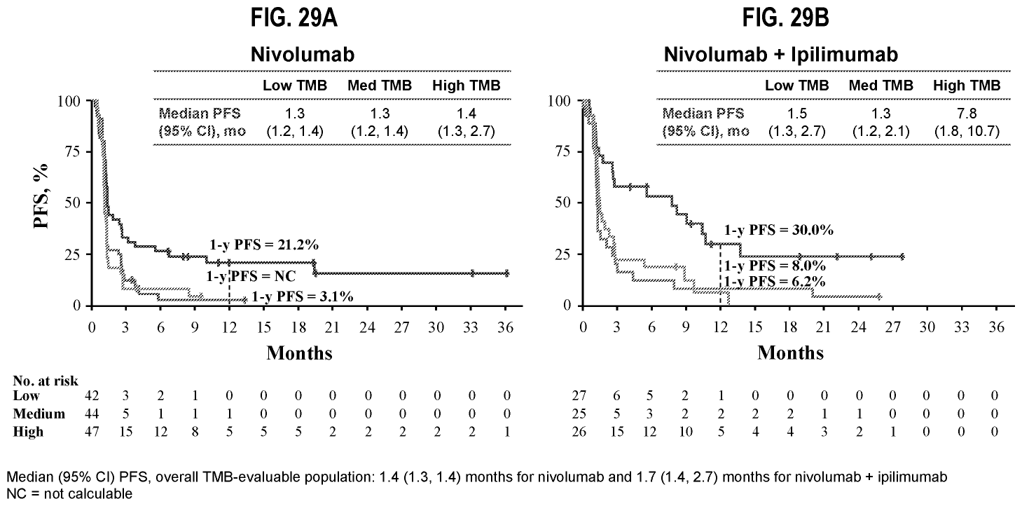

In addition, subjects with a high TMB showed increased PFS following treatment with nivolumab monotherapy (FIG. 29A) or nivolumab/ipilimumab combination therapy (FIG. 29B) as compared to subjects having a low or medium TMB. For nivolumab monotherapy, the average PFS was about 1.3% for low TMB and medium TMB subjects and about 1.4% for high TMB subjects, and the PFS at 1 year was 21.2% for high TMB subjects compared to only 3.15 for medium TMB (FIG. 29A). For nivolumab/ipilimumab combination therapy, the average PFS was about 1.5% for low TMB subjects, 1.3% for medium TMB subjects, and about 7.8% for high TMB subjects, and the PFS at 1 year was about 30% for high TMB subjects compared to about 8.0% and 6.2% for medium and low TMB subjects, respectively (FIG. 29B).

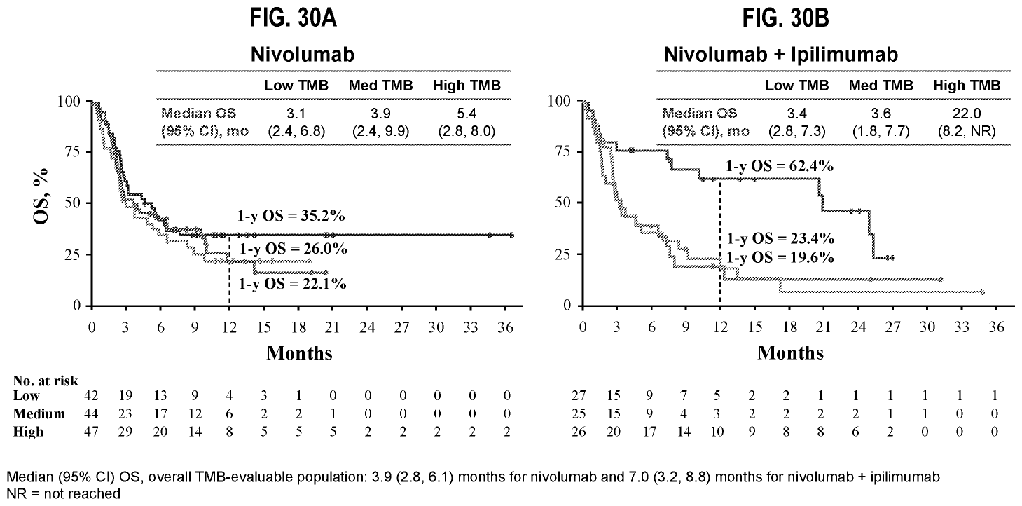

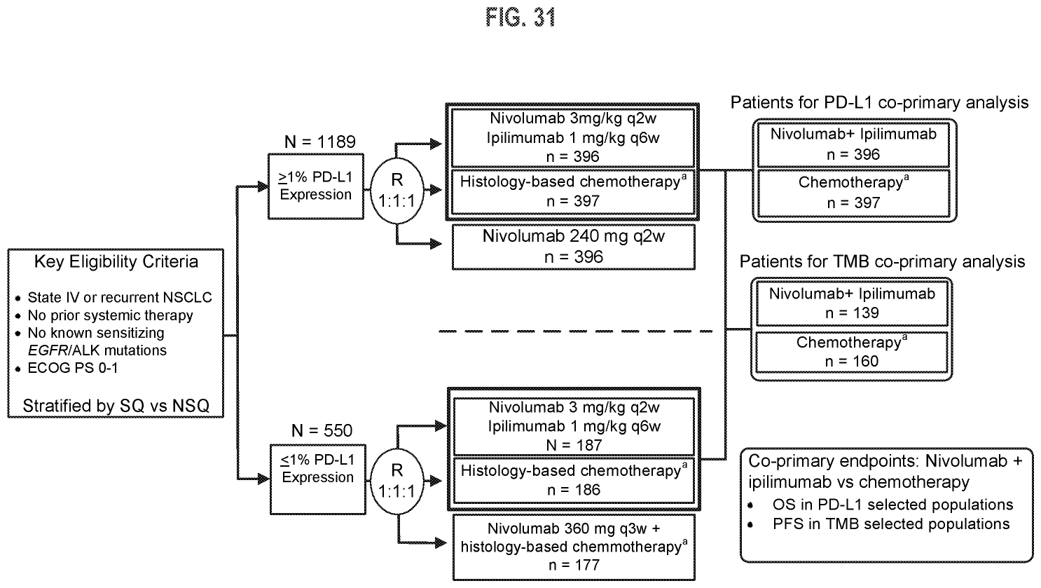

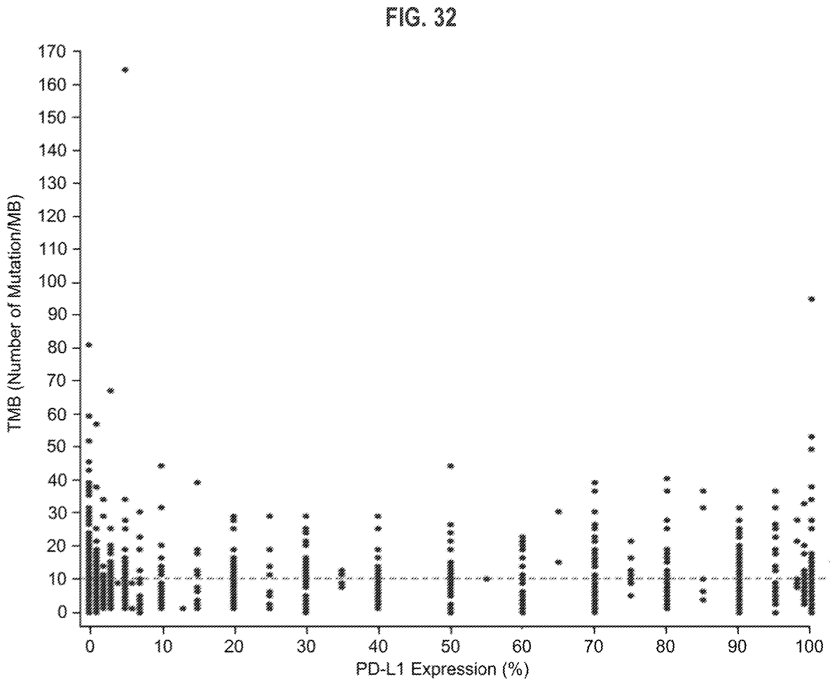

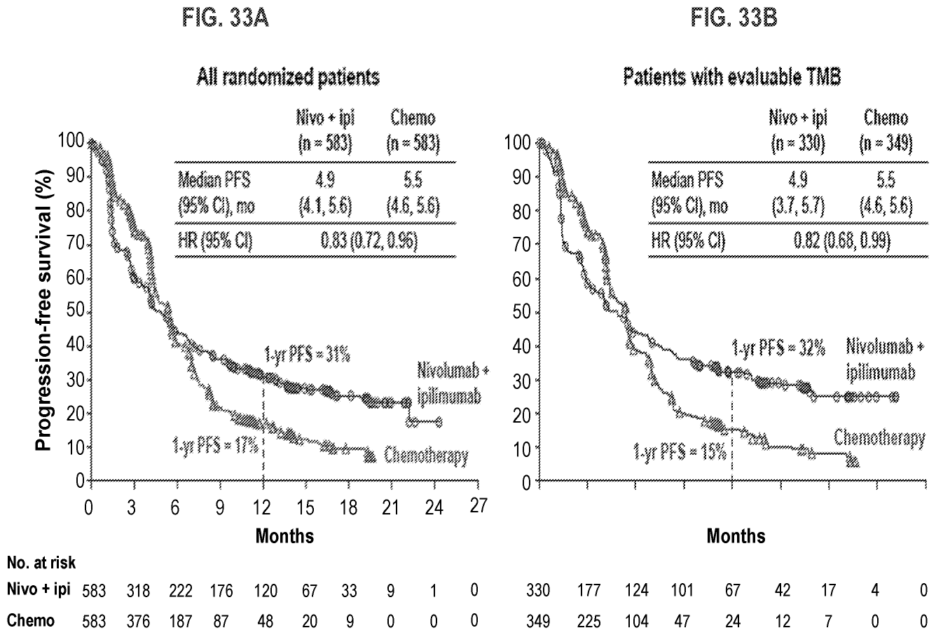

Similarly, subjects with a high TMB showed increased OS following treatment with nivolumab monotherapy (FIG. 30A) or nivolumab/ipilimumab combination therapy (FIG. 30B) as compared to subjects having a low or medium TMB. For nivolumab monotherapy, the median OS was about 3.1% for low TMB subjects, about 3.9% for medium TMB subjects, and about 5.4% for high TMB subjects, and the OS at 1 year was 35.2% for high TMB subjects compared to about 26.0% for medium TMB and 22.1% for low TMB subjects (FIG. 30A). For nivolumab/ipilimumab combination therapy, the median OS was about 3.4% for low TMB subjects, 3.6% for medium TMB subjects, and about 22% for high TMB subjects, and the OS at 1 year was about 62.4% for high TMB subjects compared to about 19.6% and 23.4% for medium and low TMB subjects, respectively (FIG. 30B).

### Example 4

Nivolumab, a programmed death (PD)-1 inhibitor, demonstrated efficacy in a single-arm phase II study in patients (pts) with metastatic or surgically unresectable urothelial carcinoma (UC) (CheckMate 275; Sharma et al. 2017). The current analysis explores the potential association between pretreatment tumor mutation burden (TMB) and response to nivolumab.

### Methods

Tumor DNA from pretreatment archival tumor tissue and matched whole blood samples was profiled by whole exome sequencing. TMB was defined as the total number of missense somatic mutations per tumor, and was evaluated as a continuous variable and by tertiles (missense count: high 167, medium 85-166, low <85). Cox models were used to explore the association between TMB and progression-free survival (PFS) and overall survival (OS); and logistic regression for objective response rate (ORR). Tumor PD-ligand 1 (PD-L1) expression was assessed by Dako PD-L1 immunohistochemistry 28-8 assay and was categorized as <1%.

### Results

139 (51%) of 270 patients had evaluable TMB. Baseline characteristics, ORR, PFS, and OS were similar between all treated patients and the TMB subgroup. ORR, PFS and OS in all patients and TMB/PD-L1 subgroups are shown in the Table 35. TMB showed a statistically significant positive association with ORR (P1/40.002) and PFS (P1/40.005), and a strong association with OS (P1/40.067), even when adjusted for baseline tumor PD-L1 expression, liver metastasis status, and serum hemoglobin. High TMB had the greatest impact on survival in patients with <1% PD-L1 expression (Table 35).

These exploratory findings suggest that TMB may enrich for response to nivolumab and may provide complementary prognostic/predictive information beyond PD-L1. Further analyses in randomized trials are warranted to define the prognostic/predictive value of TMB in the context of other biomarkers in UC patients treated with immunotherapy.

TABLE 35
ORR, PFS, and OS: All patients and TMB/PD-L1 subgroups.

| | All Pts | | TMB Subgroup | | TMB High | | TMB Medium | | TMB Low | |
|---|---|---|---|---|---|---|---|---|---|---|
| | N ¼ | | N ¼ | | N ¼ | | N ¼ | | N ¼ | |
| | 270 | | 139 | | 47 | | 46 | | 46 | |
| ORR, % | 20.0 | | 20.1 | | 31.9 | | 17.4 | | 10.9 | |
| PFS, month | 2.00 | | 2.00 | | 3.02 | | 1.87 | | 1.91 | |
| median | | | | | | | | | | |
| (95% CI) | (1.87- | | (1.87- | | (1.87- | | (1.68- | | (1.84- | |
| | 2.63) | | 3.02) | | NR) | | 3.65) | | 3.15) | |
| OS, months | 8.57 | | 7.23 | | 11.63 | | 9.66 | | 5.72 | |
| median | | | | | | | | | | |
| (95% CI) | (6.05- | | (5.72- | | (5.82- | | (4.76- | | (4.21- | |
| | 11.27) | | 11.63) | | NR) | | NR) | | 11.30) | |
| | PD-L1 | PD-L1 | PD-L1 | PD-L1 | PD-L1 | PD-L1 | PD-L1 | PD-L1 | PD-L1 | PD-L1 |
| | <1% | 1% | <1% | 1% | <1% | 1% | <1% | 1% | <1% | 1% |
| | N ¼ | N ¼ | N ¼ | N ¼ | N ¼ | N ¼ | N ¼ | N ¼ | N ¼ | N ¼ |
| | 146 | 124 | 69 | 70 | 23 | 24 | 21 | 25 | 25 | 23.8 |
| ORR, % | 15.8 | 25.0 | 17.4 | 22.9 | 30.4 | 33.3 | 23.8 | 12.0 | 0 | 23.8 |
| PFS, month | 1.87 | 3.53 | 1.87 | 2.30 | 3.02 | 3.52 | 1.77 | 1.94 | 1.77 | 3.12 |
| median | | | | | | | | | | |
| (95% CI) | (1.77- | (1.94- | (1.71- | (1.87- | (1.81- | (1.87- | (1.54- | (1.68- | (1.68- | (1.87- |
| | 2.04) | 3.71) | 3.02) | 3.71) | NR) | NR) | 5.78) | 3.71) | 2.10) | 7.23) |
| OS, months | 5.95 | 11.63 | 5.68 | 10.28 | NR | 10.60 | 4.53 | 11.30 | 4.96 | 8.57 |
| median | | | | | | | | | | |
| (95% CI) | (4.37- | (9.10- | (4.40- | (6.05- | (4.70- | (5.82- | (2.23- | (5.85- | (2.92- | (4.21- |
| | 8.08) | NR) | NR) | NR) | NR) | NR) | NR) | NR) | NR) | NR) |

### Example 5: Nivolumab Plus Ipilimumab in High Tumor Mutational Burden in Non-Small Cell Lung Cancer

Nivolumab+ ipilimumab demonstrated promising efficacy in a phase 1 NSCLC study, and tumor mutational burden (TMB) has emerged as a potential biomarker of benefit. This trial is an open-label, multi-part phase 3 study of first-line nivolumab and nivolumab-based combinations in biomarker-selected NSCLC populations. We report results from part 1 on the co-primary endpoint of progression-free survival (PFS) with nivolumab+ipilimumab versus chemotherapy in patients with high TMB (≥10 mutations/Mb). The study continues for the co-primary endpoint of overall survival in PD-L1-selected patients.

Patients had chemotherapy-naive, stage IV or recurrent NSCLC. Those with ≥1% tumor PD-L1 expression were randomized 1:1:1 to nivolumab+ipilimumab, nivolumab, or chemotherapy; those with <1% tumor PD-L1 expression were randomized 1:1:1 to nivolumab+ipilimumab, nivolumab+chemotherapy, or chemotherapy. TMB was determined using FOUNDATIONONE® CDX™.

PFS in patients with high TMB (≥10 mutations/Mb) was significantly longer with nivolumab+ipilimumab versus chemotherapy (HR, 0.58; 97.5% CI, 0.41-0.81; P=0.0002); 1-year PFS rates were 43% and 13%, and median PFS (95% CI) was 7.2 (5.5-13.2) and 5.5 (4.4-5.8) months, respectively. Objective response rates were 45.3% and 26.9%, respectively. Benefit of nivolumab+ipilimumab versus chemotherapy was broadly consistent within subgroups, including those with ≥1% and <1% PD-L1 expression. Grade 3-4 treatment-related adverse events rates were 31% and 36%, respectively.

PFS improved significantly with first-line nivolumab+ipilimumab versus chemotherapy in NSCLC with TMB≥10 mutations/Mb, irrespective of PD-L1 expression. The results validate the benefit of nivolumab+ipilimumab in NSCLC and the role of TMB as a biomarker for patient selection.

### Selection of Patients

Fresh or archival tumor-biopsy specimens obtained within 6 months before enrollment (and without the patient receiving any intervening systemic anti-cancer therapy) were tested for PD-L1 by a centralized laboratory with the use of the anti-PD-L1 antibody (28-8 antibody). Hanna, N., et al. J Oncol Pract 13:832-7 (2017).

Adult patients with PD-L1-histologically confirmed squamous or nonsquamous stage IV/recurrent NSCLC and Eastern Cooperative Oncology Group (ECOG) performance status (Oken M. M., et al. Am J Clin Oncol 5:649-55 (1982)) of 0 or 1 who had received no prior systemic anticancer therapy as primary therapy for advanced or metastatic disease were eligible for the study. See FIG. 31. All patients underwent imaging to screen for brain metastases. Patients with known EGFR mutations or ALK translocations sensitive to targeted therapy, an autoimmune disease, or untreated central nervous system metastases were excluded. Patients with central nervous system metastases were eligible if they were adequately treated and had neurologically returned to baseline for ≥2 weeks before randomization.

As additional inclusion and exclusion criteria, prior adjuvant or neoadjuvant chemotherapy or prior definitive chemoradiation for locally advanced disease was allowed up to 6 months before enrollment. Prior palliative radiotherapy to non-central nervous system lesions must have been completed ≥2 weeks before randomization. Patients had to be off glucocorticoids or on stable or decreasing doses of ≤10 mg daily prednisone (or equivalent) for ≥2 weeks before randomization.

### Study Design and Treatment

The instant study was a multi-part phase 3 trial designed to evaluate different nivolumab-based regimens vs. chemotherapy in distinct patient populations. For a period of 16 months, patients with ≥1% and <1% tumor PD-L1 expression were enrolled contemporaneously at the same centers (FIG. 31) Patients with ≥1% PD-L1 expression were randomized (1:1:1), stratified by tumor histology (squamous versus nonsquamous NSCLC), to (i) nivolumab 3 mg/kg every 2 weeks plus ipilimumab 1 mg/kg every 6 weeks, (ii) histology-based platinum-doublet chemotherapy every 3 weeks for up to 4 cycles, or (iii) nivolumab 240 mg every 2 weeks. Patients with <1% PD-L1 expression were randomized (1:1:1), stratified by tumor histology, to (i) nivolumab 3 mg/kg every 2 weeks plus ipilimumab 1 mg/kg every 6 weeks, (ii) histology-based platinum-doublet chemotherapy every 3 weeks for up to 4 cycles, or (iii) nivolumab 360 mg plus histology-based platinum-doublet chemotherapy every 3 weeks for up to 4 cycles. Patients with nonsquamous NSCLC with stable disease or response after 4 cycles of chemotherapy or chemotherapy with nivolumab could continue with maintenance pemetrexed or pemetrexed plus nivolumab. All treatments continued until disease progression, unacceptable toxicity, or completion per protocol (up to 2 years for immunotherapy). Crossover between treatment arms within the study was not permitted.

Of 2877 patients enrolled in part 1 of the trial, 1739 underwent randomization. Of the 1138 patients who were not randomized, 909 patients no longer met the study criteria (common reasons included EGFR/ALK mutations identified, decline in ECOG PS, untreated brain metastases, and non-evaluable PD-L1 expression), 88 patients withdrew consent, 40 patients died, 33 patients had adverse events (unrelated to study drug), 6 patients were lost to follow-up, and 62 patients were excluded for other reasons.

As shown in Tables 36 and 37, the baseline characteristics in all randomized and TMB-evaluable patients were similar and balanced between treatment arms.

TABLE 36
Baseline Characteristics of All Randomized Patients.

| | All randomized patients | | | |
|---|---|---|---|---|
| | Nivolumab + | | Chemo- | |
| | Ipilimumab | Nivolumab | therapy | Total |
| | (n = 583) | (n = 396) | (n = 583) | (N = 1739) |
| Median age, years | 64 | 64 | 64 | 64 |
| Female, % | 33 | 31 | 34 | 32 |
| ECOG PS, % | | | | |
| 0 | 35 | 36 | 33 | 34 |
| 1 | 65 | 64 | 66 | 65 |
| ≥2 | >1 | 0 | 1 | <1 |
| Not reported | 0 | <1 | <1 | <1 |
| Smoking status, % | | | | |
| Current/former | 85 | 86 | 86 | 85 |
| smoker | | | | |
| Never smoker | 14 | 13 | 13 | 13 |
| Unknown | 1 | 1 | 1 | 1 |
| Histology, % | | | | |
| Squamous | 28 | 30 | 28 | 28 |
| Non-squamous | 72 | 70 | 72 | 72 |
| PD-L1 expression, | | | | |
| % | | | | |
| <1% | 32 | 0 | 32 | 32 |
| ≥1% | 68 | 100 | 68 | 68 |

ECOG PS = Eastern Cooperative Oncology Group performance status;
PD-L1 = programmed death ligand 1.

TABLE 37
Baseline Characteristics of All TMB-evaluable Patients.

| | TMB evaluable patients | | | |
|---|---|---|---|---|
| | Nivolumab + | | Chemo- | |
| | Ipilimumab | Nivolumab | therapy | Total |
| | (n = 330) | (n = 228) | (n = 349) | (N = 1004) |
| Median age, years | 64 | 64 | 64 | 64 |
| Female, % | 34 | 31 | 36 | 33 |
| ECOG PS, % | | | | |
| 0 | 33 | 32 | 34 | 33 |
| 1 | 67 | 67 | 65 | 67 |
| ≥2 | <1 | 0 | 1 | <1 |
| Not reported | 0 | <1 | <1 | <1 |
| Smoking status, % | | | | |
| Current/former | 86 | 86 | 87 | 87 |
| smoker | | | | |
| Never smoker | 12 | 12 | 11 | 12 |
| Unknown | 2 | 1 | 1 | 1 |
| Histology, % | | | | |
| Squamous | 28 | 29 | 32 | 29 |
| Non-squamous | 72 | 71 | 68 | 71 |
| PD-L1 expression, | | | | |
| % | | | | |
| <1% | 27 | 0 | 31 | 29 |
| ≥1% | 73 | 100 | 69 | 71 |

ECOG PS = Eastern Cooperative Oncology Group performance status

### Tumor Mutation Burden Analysis

TMB was assessed in archival or fresh formalin-fixed, paraffin-embedded tumor samples using the validated assay FOUNDATIONONE® CDX™, which employs next generation sequencing to detect substitutions, insertions and deletion (indels), and copy number alterations in 324 genes and select gene rearrangements. Ettinger, D. S., et al. J Natl Compr Canc Netw, 15:504-35 (2017). Independent reports have demonstrated concordance between TMB estimated from whole exome sequencing (WES) and TMB estimated from targeted next generation sequencing (NGS). See Szustakowski J., et al. Evaluation of tumor mutation burden as a biomarker for immune checkpoint inhibitor efficacy: A calibration study of whole exome sequencing with FoundationOne®. Presented at the American Association for Cancer Research 2018 Annual Meeting; 2018; Chicago, Illinois; Zehir A, et al. Nat Med 2017; 23:703-713; Rizvi H., et al., J Clin Oncol 2018; 36:633-41. TMB was calculated according to previously defined methods. Reck, M., et al., N Engl J Med, 375:1823-33 (2016). Briefly, TMB was defined as the number of somatic, coding, base substitution and short indels per megabase of genome examined. All base substitutions and indels in the coding region of targeted genes, including synonymous mutations, were filtered for both oncogenic driver events according to COSMIC and germline status according to dbSNP and ExAC databases, in addition to a private database of rare germline events compiled in the Foundation Medicine clinical cohort. Additional filtering based upon a computational assessment of germline status using the SGZ (somatic-germline-zygosity) tool was also performed. Aguiar, P. N., et al., ESMO Open, 2: e000200 (2017).

As shown in Table 38, of all randomized patients (N=1739), 1649 (95%) had tumor samples for TMB assessment, and 1004 (58%) had valid TMB data for TMB-based efficacy analyses.

TABLE 38
Sample Size Throughout
TMB Determination
Patients, n (%)

| | |
|---|---|
| Randomized^{a} | 1739 (100) |
| Samples available | 1649 (95) |
| TMB-evaluable samples^{b} | 1004 (58) |

^{a}Randomized patients include those from all treatment arms in Part 1 (nivolumab + ipilimumab, nivolumab, chemotherapy, and nivolumab + chemotherapy arms)
^{b}A pre-analytical quality control check was performed on all samples to flag inaccuracies comprised of but not limited to incorrect requisitions, receipt of insufficient sample, and duplicate samples. The FOUNDATIONONE ® CDX ™ assay employs comprehensive quality control criteria, including the following critical characteristics: tumor purity, DNA sample size, tissue sample size, library construction size, and hybrid capture yields.

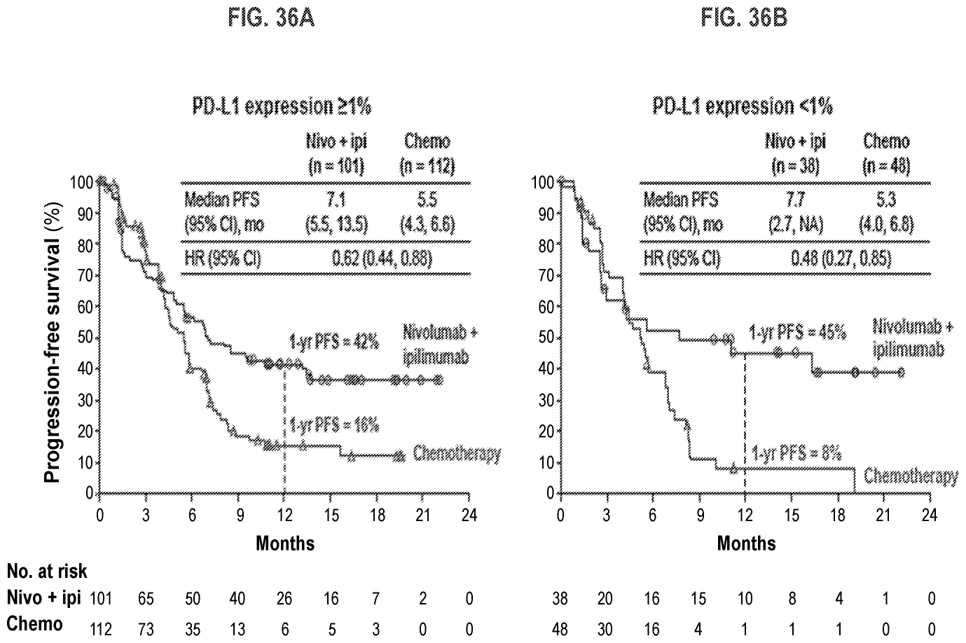

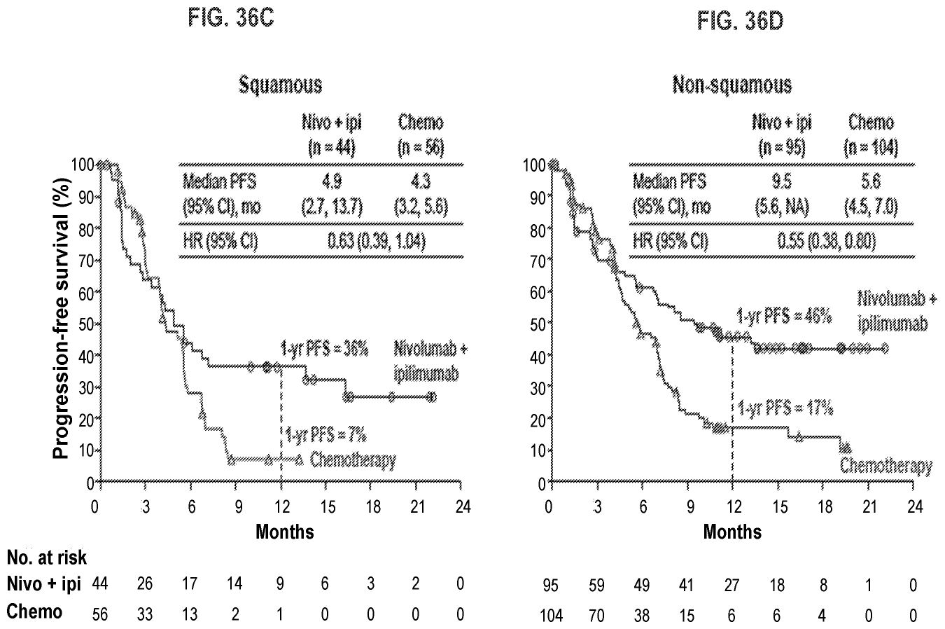

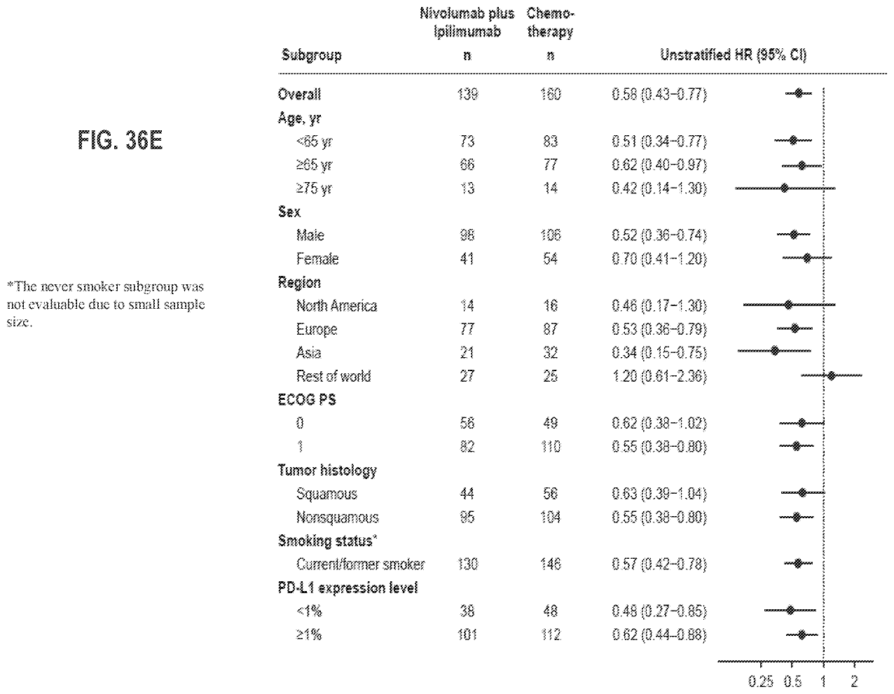

Of all TMB-evaluable patients across all treatment arms, 444 (44%) had TMB≥10 mutations/Mb, including 139 patients randomized to nivolumab plus ipilimumab and 160 patients randomized to chemotherapy. As shown in Table 39, baseline characteristics between the two treatment groups were well balanced, including distribution of PD-L1 expression. In the TMB-evaluable population, there was no correlation between TMB and PD-L1 expression. FIGS. 36A and 36B.

TABLE 39
Baseline Characteristics of Patients with TMB ≥10 mutations/Mb.

| | Nivolumab | |
|---|---|---|
| | plus | |
| | Ipilimumab | Chemotherapy |
| Characteristic | (n = 139) | (n = 160) |
| Age, years | | |
| Median | 64 | 64 |
| Range | 41-87 | 29-80 |
| Age category, n (%) | | |
| <65 years | 73 (53) | 83 (52) |
| ≥65 to <75 years | 53 (38) | 63 (39) |
| ≥75 years | 13 (9) | 14 (9) |
| Sex, n (%) | | |
| Male | 98 (71) | 106 (66) |
| Female | 41 (29) | 54 (34) |
| Region, n (%) | | |
| North America | 14 (10) | 16 (10) |
| Europe | 77 (55) | 87 (54) |
| Asian | 21 (15) | 32 (20) |
| Rest of World | 27 (19) | 25 (16) |
| ECOG performance-status score, n (%) | | |
| 0 | 56 (40) | 49 (31) |
| 1 | 82 (59) | 110 (69) |
| ≥2 | 1 (1) | 1 (1) |
| Smoking status, n (%) | | |
| Current/Former Smoker | 130 (94) | 146 (91) |
| Never smoker | 7 (5) | 11 (7) |
| Unknown | 2 (1) | 3 (2) |
| Tumor histology, n (%) | | |
| Squamous cell carcinoma | 45 (32) | 55 (34) |
| Nonsquamous cell carcinoma | 94 (68) | 105 (66) |
| PD-L1 expression level, n (%) | | |
| <1% | 38 (27) | 48 (30) |
| ≥1% | 101 (73) | 112 (70) |

At a minimum follow-up of 11.2 months, 17.7°% and 5.6°% of patients treated with nivolumab plus ipilimumab and chemotherapy, respectively remained on treatment. See Table 40.

TABLE 40
End-of-Treatment Summary.

| | All Treated Patients | | TMB ≥10 mutations/Mb | |
|---|---|---|---|---|
| | Nivolumab + | Chemo- | Nivolumab + | Chemo- |
| | Ipilimumab | therapy | Ipilimumab | therapy |
| | n = 576 | n = 570 | n = 135 | n = 159 |
| Patients continuing | 102 (17.7) | 32 (5.6) | 33 (24.2) | 5 (3.1) |
| in the treatment | | | | |
| period, n (%) | | | | |
| Patients not | 474 (82.3) | 538 (94.4) | 102 (75.6) | 154 (96.9) |
| continuing | | | | |
| in the treatment | | | | |
| period, n (%) | | | | |
| Reason for not | | | | |
| continuing in the | | | | |
| treatment period, | | | | |
| n (%) | | | | |
| Disease progression | 285 (49.5) | 279 (48.9) | 51 (37.8) | 75 (47.2) |
| Study drug toxicity | 108 (18.8) | 51 (8.9) | 35 (25.9) | 14 (8.8) |
| Completed required | 2 (0.3) | 126 (22.1) | 0 | 42 (26.4) |
| treatment | | | | |
| Death | 6 (1.0) | 2 (0.4) | 1 (0.7) | 0 |
| Adverse event | 39 (6.8) | 35 (6.1) | 7 (5.2) | 9 (5.7) |
| unrelated | | | | |
| to study drug | | | | |
| Patient request to | 9 (1.6) | 19 (3.3) | 3 (2.2) | 8 (5.0) |
| discontinue | | | | |
| Patient withdrew | 8 (1.4) | 6 (1.1) | 1 (0.7) | 1 (0.6) |
| consent | | | | |
| Lost to follow-up | 1 (0.2) | 1 (0.2) | 0 | 0 |
| Maximum clinical | 3 (0.5) | 0 | 1 (0.7) | 0 |
| benefit | | | | |
| Lack of compliance | 1 (0.2) | 2 (0.4) | 0 | 1 (0.6) |
| Patient no | 1 (0.2) | 1 (0.2) | 0 | 0 |
| longer meets | | | | |
| study criteria | | | | |
| Other | 11 (1.9) | 10 (1.8) | 3 (2.2) | 2 (1.3) |
| Not reported | 0 | 6 (1.1) | 0 | 2 (1.3) |

Of patients s assigned to chemotherapy, 28.1°% received subsequent immunotherapy. See Table 41.

TABLE 41
Subsequent Systemic Therapies in Patients
With TMB ≥10 mutations/Mb.^{a}

| | Nivolumab + | |
|---|---|---|
| | Ipilimumab | Chemotherapy |
| Patients, n (%) | n = 139 | n = 160 |
| Any subsequent systemic therapy | 23 (16.5) | 69 (43.1) |
| Immunotherapy | 3 (2.2) | 45 (28.1) |
| Anti-PD-1 | 3 (2.2) | 42 (26.3) |
| Nivolumab | 3 (2.2) | 36 (22.5) |
| Pembrolizumab | 0 | 6 (3.8) |
| Anti-PD-L1 (atezolizumab) | 0 | 1 (0.6) |
| Anti-CTLA-4 (ipilimumab) | 0 | 5 (3.1)^{b} |
| Other immunotherapy | 0 | 2 (1.3) |
| Targeted therapy | 2 (1.4) | 3 (1.9) |
| Chemotherapy | 22 (15.8) | 33 (20.6) |

^{a}At the time of database lock, 24% of patients treated with nivolumab + ipilimumab and 3% of those treated with chemotherapy were still on treatment.
^{b}All 5 patients received ipilimumab in combination with nivolumab.

The median duration of therapy was 4.2 months (range, 0.03 to 24.0+) with nivolumab plus ipilimumab and 2.6 months (range, 0.03 to 22.1+) with chemotherapy. The median number of doses of nivolumab (every 2 weeks) and ipilimumab (every 6 weeks) received as combination therapy was 9 (range, 1 to 53) and 3 (range, 1 to 18), respectively.

Among patients with high TMB (≥10 mutations/Mb), 24.2% treated with nivolumab plus ipilimumab and 3.1% treated with chemotherapy were continuing treatment at the time of database lock; the most common reason for discontinuing treatment was disease progression (37.8% and 47.2%, respectively), study drug toxicity (25.9% and 8.8%, respectively), and completion of required treatment among patients in the chemotherapy group (26.4% vs. 0% for patients treated with nivolumab plus ipilimumab)

### Endpoints and Assessments:

Part 1 of this study had two co-primary endpoints. One co-primary endpoint was progression-free survival (PFS), which was assessed by blinded independent central review, with nivolumab plus ipilimumab vs. chemotherapy in a TMB-selected patient population. Based on previous findings (Ramalingam S S, et al. Tumor mutation burden (TMB) as a biomarker for clinical benefit from dual immune checkpoint blockade with nivolumab (nivo)+ipilimumab (ipi) in first-line (1L) non-small cell lung cancer (NSCLC): identification of TMB cutoff from CheckMate 568. Presented at the American Association for Cancer Research 2018 Annual Meeting; 2018; Chicago, Illinois.), a predefined TMB cutoff of ≥10 mutations/Mb was selected for preplanned analysis of the co-primary endpoint. The second co-primary endpoint was overall survival (OS) with nivolumab plus ipilimumab vs. chemotherapy in a PD-L1-selected patient population.

As shown in Table 42, secondary endpoints in TMB-selected patient populations included PFS with nivolumab vs. chemotherapy in patients with TMB≥13 mutations/Mb and ≥1% PD-L1 expression and OS with nivolumab plus ipilimumab vs. platinum-doublet chemotherapy in patients with TMB≥10 mutations/Mb.

TABLE 42
Hierarchical Hypothesis Testing in TMB-Selected Patients.

| Hierarchy | Endpoint | Population | Comparison |
|---|---|---|---|
| 1 | Primary endpoint: | TMB ≥10 | Nivolumab + |
| | PFS | mutations/Mb | Ipilimumab |
| | Alpha = 0.25 | | vs |
| | | | Chemotherapy |
| 2 | Secondary endpoint: | TMB ≥13 mutations/Mb | Nivolumab |
| | PFS | and ≥1% tumor | vs |
| | | PD-L1 expression | Chemotherapy |
| 3 | Secondary | TMB ≥10 | Nivolumab + |
| | endpoint: | mutations/Mb | Ipilimumab |
| | OS | | vs |
| | | | Chemotherapy |
| 4 | Secondary endpoint: | TMB ≥13 mutations/Mb | Nivolumab |
| | OS | and ≥1% tumor | vs |
| | | PD-L1 expression | Chemotherapy |

Exploratory endpoints: ORR, PFS for all arms, safety

PFS = progression-free survival;
ORR = objective response rate;
OS = overall survival

The TMB cutoff of ≥13 mutations/Mb for the secondary endpoint of PFS with nivolumab versus chemotherapy was based on analyses from the previous studies, including a bridging study converting whole exome sequencing data to FOUNDATIONONE® CDX™ data. See Carbone et al. N Engl J Med 2017; 376:2415-26; Szustakowski et al., Evaluation of tumor mutation burden as a biomarker for immune checkpoint inhibitor efficacy: A calibration study of whole exome sequencing with FoundationOne®. In: American Association for Cancer Research 2018 Annual Meeting. Chicago, Illinois; 2018. Overall response rates (ORR), duration of response, and safety were exploratory endpoints. Adverse events were graded according to the National Cancer Institute Common Terminology Criteria for Adverse Events, version 4.0. PD-L1 was determined as previously described. See Labeling: PD-L1 IHC 28-8 pharmDx. Dako North America, 2016 (Accessed Oct. 20, 2016, at accessdata.fda.gov/cdrh_docs/pdf15/P150027c.pdf.)

TMB, defined as the number of somatic, coding, base substitutions and short insertions and deletions (indels) per megabase of genome examined, was determined using the FOUNDATIONONE® CDX™ assay. See, e.g., FOUNDATIONONE® CDX™ Foundation Medicine, 2018. (Accessed Feb. 8, 2018, at foundationmedicine.com/genomic-testing/foundation-one-cdx.); Chalmers et al., Analysis of 100,000 human cancer genomes reveals the landscape of tumor mutational burden. Genome Med 2017; 9:34; and Sun J X, He Y, Sanford E, et al. The mutation count following application of various filters was divided by the region counted (0.8 Mb) to yield mutations/Mb.

For the co-primary endpoint of PFS with nivolumab plus ipilimumab vs. chemotherapy in patients with TMB≥10 mutations/Mb, it was estimated that a sample size of at least 265 patients with approximately 221 events of death or disease progression would provide 80% power to detect a hazard ratio of 0.66 favoring nivolumab plus ipilimumab vs. chemotherapy, with a two-sided type 1 error of 0.025, by means of a two-sided log-rank test. Hazard ratios of PFS with associated two-sided confidence intervals were estimated using an unstratified Cox proportional hazard model, with treatment group as a single covariate. A multivariate analysis was prespecified in patients with TMB≥10 mutations/Mb to assess the influence of known prognostic baseline factors on PFS. Estimates of hazard ratios with corresponding two-sided 97.5% CI were computed for primary and secondary comparisons specified in the hierarchical hypothesis testing in TMB-selected patients (see Table 42, above); for all other estimates two-sided 95% CI were computed that should not be used to infer differences in treatment effects. Survival curves were estimated using Kaplan-Meier methodology.

In conclusion, this study met its co-primary endpoint, and the results may establish two new standards of care in advanced NSCLC. First, all treatment-naïve NSCLC patients should be tested for TMB as the results validate the role of TMB as an important and independent biomarker. Second, this study introduces nivolumab plus ipilimumab as a new first-line treatment option for patients with high TMB≥10 mutations/Mb. These results provide a more personalized approach to treating lung cancer, by offering effective first-line, chemotherapy-sparing combination immunotherapy to patients who are most likely to receive durable benefit, while preserving effective second-line options. The use of TMB as a predictive biomarker for patients with NSCLC provides an example of precision medicine, tailoring treatment to those patients who will most likely benefit from combination immunotherapy.

### All Randomized Patients

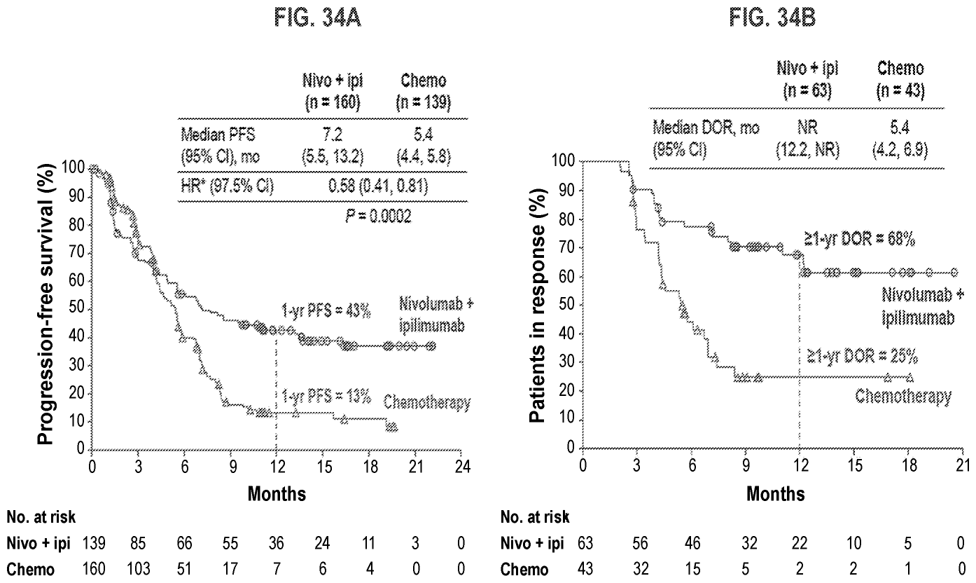

In all randomized patients (irrespective of PD-L1 expression), PFS improved with nivolumab plus ipilimumab vs. chemotherapy (hazard ratio [HR], 0.83; 95%, 0.72 to 0.96), with 1-year PFS rates of 31% versus 17%. The median PFS was 4.9 months (95% CI, 4.1 to 5.6) with nivolumab plus ipilimumab and 5.5 months (95% CI, 4.6 to 5.6) with chemotherapy. Similar benefit with nivolumab plus ipilimumab versus chemotherapy was seen among TMB-evaluable patients (HR, 0.82; 95% CI, 0.68 to 0.99), with 1-year PFS rates of 32% versus 15%; the median PFS was 4.9 months (95% CI, 3,7 to 5.7) and 5.5 months (95% CI, 4.6 to 5.6), respectively. See FIGS. 34A and 34B.

Patients with High TMB (≥10 Mutations/Mb) v. Low TMB

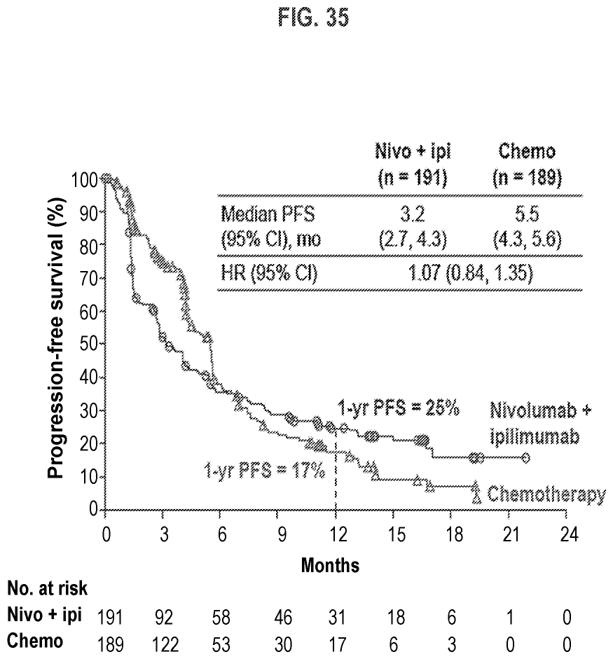

Analysis of the co-primary endpoint in patients with high TMB (≥10 mutations/Mb) showed significant improvement of PFS with nivolumab plus ipilimumab versus chemotherapy (HR, 0.58; 97.5% CI, 0.41 to 0.81; P=0.0002) with the 1-year PFS rates of 43% versus 13% with chemotherapy, and median PFS was 7.2 months (95% CI, 5.5 to 13.2) and 5.5 months (95% CI, 4.4 to 5.8), respectively. FIG. 34A. In a prespecified multivariate analysis of PFS in patients with TMB≥10 mutations/Mb, the treatment effect of nivolumab plus ipilimumab vs chemotherapy adjusted for baseline PD-L1 expression level (≥1%, <1%), gender, tumor histology (squamous, non-squamous) and ECOG PS (0, ≥1) was consistent with the primary PFS analysis (HR, 0.57; 95% CI, 0.40 to 0.80, multivariate Cox model P=0.0002). In patients with TMB<10 mutations/Mb, no improvement of PFS was observed with nivolumab plus ipilimumab versus chemotherapy (HR, 1.07; 95% CI, 0.84 to 1.35); median PFS was 3.2 months (95% CI, 2.7 to 4.3) with nivolumab plus ipilimumab and 5.5 months (95% CI, 4.3 to 5.6) with chemotherapy. See FIG. 35.

The objective response rate was 45.3% with nivolumab plus ipilimumab and 26.9% with chemotherapy (Table 43) Eisenhauer, E. A., et al. Eur J Cancer, 45:228-47 (2009). The percentage of responders with ongoing who still were in response after 1-year was 68% for nivolumab plus ipilimumab and 25% for chemotherapy (FIG. 34B).

TABLE 43
Tumor Response in Patients with TMB ≥10 mutations/Mb.

| | Nivolumab plus | |
|---|---|---|
| | Ipilimumab | Chemotherapy |
| Variable | (n = 139) | (n = 160) |
| Objective response† | | |
| No. of patients | 63 | 43 |
| % of patients (95% CI) | 45.3 (36.9-54.0) | 26.9 (20.2-34.4) |
| Difference (95% CI) | 18.4 (7.6-28.8) | |
| Best overall response-no. (%) | | |
| Complete response | 5 (3.6) | 1 (0.6) |
| Partial response | 58 (41.7) | 42 (26.3) |
| Stable disease | 37 (26.6) | 88 (55.0) |
| Progressive disease | 22 (15.8) | 19 (11.9) |
| Could not be determined | 17 (12.2) | 10 (6.3) |
| Time to objective response-mo‡§ | | |
| Median | 2.7 | 1.5 |
| Range | 1.2-9.5 | 1.2-6.9 |
| Duration of objective response-mo‡¶ | | |
| Median | NR | 5.4 |
| Range | 2.1-20.5+ | 2.6-18.1+ |
| 1-year response rate, % | | |
| Estimate | 68 | 25 |
| 95% confidence interval | 54-78 | 12-40 |

*Data are based on a Jan. 24, 2018, database lock.
†Objective response was assessed according to the Response Evaluation Criteria in Solid Tumors, version 1.1,27 by blinded independent central review. The 95% confidence interval (CI) is based on the Clopper-Pearson method. Unweighted difference in objective response rates between treatment groups was determined by the method of Newcombe.
‡The analysis was performed with data from all the patients who had a response (63 patients in the nivolumab group and 43 in the chemotherapy group).
§The time to response was defined as the time from randomization to the date of first documented complete or partial response.
¶Results were calculated with the use of the Kaplan-Meier method. The duration of response was defined as the time between the date of first response and the date of first documented event of progression, death, or last tumor assessment that was evaluated before subsequent therapy (data-censoring date).
NR denotes not reached.

Selected Subgroups in Patients with High TMB (≥10 Mutations/Mb)

Subgroup analysis by PD-L1 status showed that PFS was improved with nivolumab plus ipilimumab vs. chemotherapy in patients with ≥1% PD-L1 expression and those with <1% PD-L1 expression. FIGS. 36A and 36B Improved PFS with nivolumab plus ipilimumab vs. chemotherapy was seen in patients with both squamous and nonsquamous tumor histology. FIGS. 36C and 36D Across most other subgroups of patients with TMB≥10 mutations/Mb, PFS was improved with nivolumab plus ipilimumab vs. chemotherapy. FIG. 36E.

### Nivolumab Monotherapy

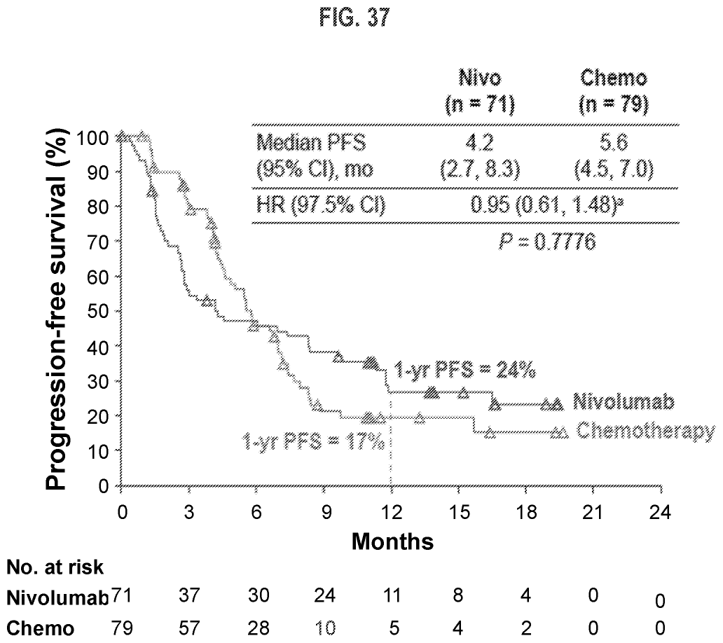

A secondary endpoint of the study was efficacy of nivolumab (n=79) vs. chemotherapy (n=71) among patients with TMB≥13 mutations/Mb and ≥1% PD-L1 expression (patients with <1% PD-L1 expression were not eligible to receive nivolumab); there was no improvement in PFS with nivolumab in this patient group (HR, 0.95; 97.5% CI, 0.61, 1.48; P=0.7776). The median PFS was 4.2 months (95% CI, 2.7 to 8.3) with nivolumab and 5.6 months (95% CI, 4.5 to 7.0) with chemotherapy. FIG. 37.

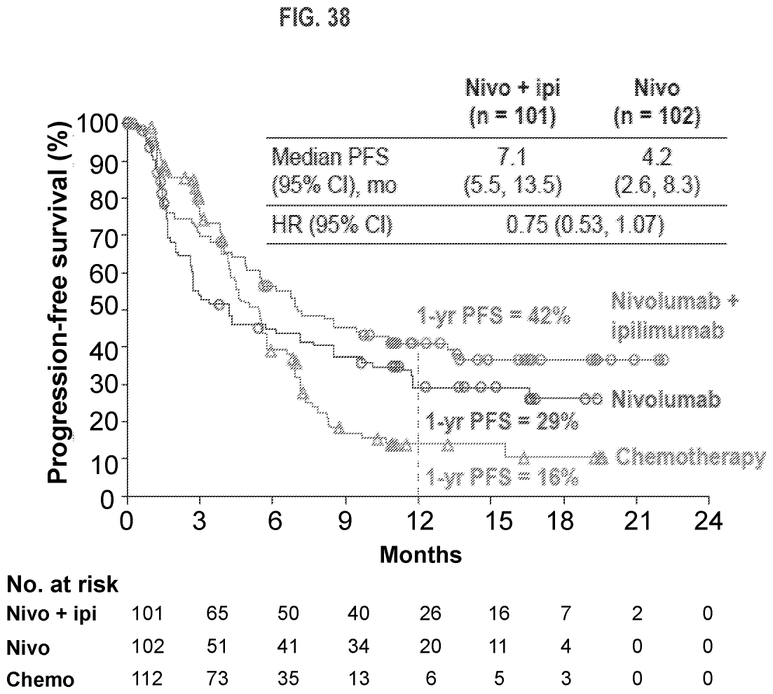

Among patients with TMB≥10 mutations/Mb and ≥1% PD-L1 expression, median PFS was 7.1 months (95% CI, 5.5 to 13.5) with nivolumab plus ipilimumab versus 4.2 months (95% CI, 2.6 to 8.3) with nivolumab monotherapy (HR, 0.75; 95% CI, 0.53 to 1.07). FIG. 38.

The results of this study demonstrate that in patients with advanced NSCLC and TMB≥10 mutations/Mb, first-line treatment with nivolumab plus ipilimumab is associated with improved PFS compared with chemotherapy. The benefit of combination immunotherapy was durable, with 43% of patients being progression free at 1 year (vs. 13% with chemotherapy) and 68% of responders having ongoing responses at 1 year (vs. 25% with chemotherapy). The benefit of nivolumab plus ipilimumab was observed in patients with ≥1% and <1% PD-L1 expression, squamous and nonsquamous histology, and was consistent across the majority of other subgroups. Although improved PFS was seen with nivolumab plus ipilimumab vs. chemotherapy in all randomized patients, TMB≥10 mutations/Mb was an effective biomarker. Benefit with nivolumab plus ipilimumab was particularly enhanced in those with high TMB while no benefit relative to chemotherapy was seen in those with low TMB (<10 mutations/Mb). Additionally, nivolumab plus ipilimumab had improved efficacy compared with nivolumab monotherapy in patients with TMB≥10 mutations/Mb, highlighting the distinct importance of dual immune-checkpoint blockade in NSCLC with TMB≥10 mutations/Mb. The study continues for the co-primary endpoint of OS in PD-L1-selected patients.

This study shows that the TMB and PD-L1 expressions were independent biomarkers. Among patients with high TMB, the benefit of nivolumab plus ipilimumab compared with chemotherapy was similar in patients with ≥1% and <1% tumor PD-L1 expression. Therefore, nivolumab plus ipilimumab represents a new, effective treatment regimen for patients with TMB≥10 mutations/Mb irrespective of PD-L1 expression.

Safety of nivolumab plus ipilimumab was consistent with previously reported data in first-line NSCLC. In a previous study, various dosing regimens of nivolumab plus ipilimumab were evaluated in 8 cohorts, and the nivolumab 3 mg/kg every 2 weeks plus ipilimumab 1 mg/kg every 6 weeks regimen was found to be well tolerated and effective. Hellmann, M. D., et al. Lancet Oncol, 18:31-41 (2017). These findings were confirmed in our large, international study, with no new safety signals observed with the combination. The rates of treatment-related select adverse events and treatment-related discontinuations were only modestly higher than those with nivolumab monotherapy, which was also well tolerated, with low rates of select adverse events.

Although the rates of treatment-related adverse events leading to discontinuation were higher with nivolumab plus ipilimumab than chemotherapy, this may in part be related to longer treatment durations and longer PFS with nivolumab plus ipilimumab.

Important questions remain regarding the role of immunotherapy/immunotherapy combinations versus immunotherapy/chemotherapy combinations, the optimal sequencing of therapies, whether TMB can identify patients who may derive benefit from immunotherapy/chemotherapy combinations, and whether an optimal TMB cutoff can be identified for PD-1/L1 monotherapy. Given that the results of our study validate the clinical utility of TMB as an important and independent biomarker, a concerted multidisciplinary effort will be necessary to ensure the availability of sufficient tumor tissue for testing and acceptable turnaround time. The 58% rate of TMB results reported in this study was mainly due to the limited availability of tumor samples of sufficient quantity or quality, a result of limited tissue requested for biomarker analysis as part of the study. In clinical practice, when the intent to test for TMB is known upfront and sufficient quantity and quality of tumor samples can be collected and submitted, successful TMB determination can be expected for 80% to 95% of patients undergoing testing.24 CheckMate 817 (NCT02869789), which will prospectively evaluate the feasibility of TMB testing for first-line nivolumab plus ipilimumab in patients with advanced NSCLC and TMB≥10 mutations/Mb, may help to identify gaps and opportunities in education to optimize the feasibility for TMB testing. Moreover, TMB is a reliable and reproducible biomarker that simultaneously provides comprehensive genomic profiling through next generation sequencing of multiple potentially therapeutically actionable cancer genes. Therefore, TMB testing leverages already routine technology to provide broadly applicable, clinically important information within a single test to guide management in first line NSCLC.

### Treatment Beyond Progression and Overall Survival Follow-Up

Treatment continuation with nivolumab or nivolumab plus ipilimumab beyond progression was permitted if the patient had investigator-assessed clinical benefit and continued to tolerate treatment. Patients were followed for overall survival every 3 months via in-person or phone contact after discontinuation of study drug treatment.

This application claims the benefit of U.S. Provisional Application Nos. 62/479,817, filed Mar. 31, 2017, and 62/582,146, filed Nov. 6, 2017, which are incorporated by reference herein in their entireties.

### REFERENCES

1. Ettinger D S, Wood D E, Akerley W, et al. NCCN Guidelines Insights: Non-Small Cell Lung Cancer, Version 4.2016. J Natl Compr Canc Netw 2016; 14:255-64.
2. Masters G A, Temin S, Azzoli C G, et al. Systemic therapy for stage IV non-small-cell lung cancer: American Society of Clinical Oncology clinical practice guideline update. J Clin Oncol 2015; 33:3488-515.
3. Scagliotti G V, Parikh P, von Pawel J, et al. Phase III study comparing cisplatin plus gemcitabine with cisplatin plus pemetrexed in chemotherapy-naive patients with advanced-stage non-small-cell lung cancer. J Clin Oncol 2008; 26:3543-51.
4. Socinski M A, Bondarenko I, Karaseva N A, et al. Weekly nab-paclitaxel in combination with carboplatin versus solvent-based paclitaxel plus carboplatin as first-line therapy in patients with advanced non-small-cell lung cancer: final results of a phase III trial. J Clin Oncol 2012; 30:2055-62.
5. Patel J D, Socinski M A, Garon E B, et al. PointBreak: a randomized phase III study of pemetrexed plus carboplatin and bevacizumab followed by maintenance pemetrexed and bevacizumab versus paclitaxel plus carboplatin and bevacizumab followed by maintenance bevacizumab in patients with stage IIIB or IV nonsquamous non-small-cell lung cancer. J Clin Oncol 2013; 31:4349-57.
6. Paz-Ares L, Mezger J, Ciuleanu T E, et al.; for the INSPIRE investigators. Necitumumab plus pemetrexed and cisplatin as first-line therapy in patients with stage IV non-squamous non-small-cell lung cancer (INSPIRE): an open-label, randomised, controlled phase 3 study. Lancet Oncol 2015; 16:328-37.
7. Thatcher N, Hirsch F R, Luft A V, et al.; for the SQUIRE investigators. Necitumumab plus gemcitabine and cisplatin versus gemcitabine and cisplatin alone as first-line therapy in patients with stage IV squamous non-small-cell lung cancer (SQUIRE): an open-label, randomised, controlled phase 3 trial. Lancet Oncol 2015; 16:763-74.
8. Zinner R G, Obasaju C K, Spigel D R, et al. PRONOUNCE: randomized, open-label, phase III study of first-line pemetrexed+carboplatin followed by maintenance pemetrexed versus paclitaxel+carboplatin+bevacizumab followed by maintenance bevacizumab in patients with advanced nonsquamous non-small-cell lung cancer. J Thorac Oncol 2015; 10:134-42.
9 Brahmer J, Reckamp K L, Baas P, et al. Nivolumab versus docetaxel in advanced squamous-cell non-small-cell lung cancer. N Engl J Med 2015; 373:123-35.
10. Borghaei H, Paz-Ares L, Horn L, et al. Nivolumab versus docetaxel in advanced nonsquamous non-small-cell lung cancer. N Engl J Med 2015; 373:1627-39.
11. Barlesi F, Steins M, Horn L, et al. Long-term outcomes with nivolumab (Nivo) vs docetaxel (Doc) in patients (Pts) with advanced (Adv) NSCLC: CheckMate 017 and CheckMate 057 2-y update. Ann Oncol 2016; 27: abstract 1215PD.
12. Gettinger S, Rizvi N A, Chow L Q, et al. Nivolumab monotherapy for first-line treatment of advanced non-small-cell lung cancer. J Clin Oncol 2016; 34:2980-7.
13. Gettinger S, Shepherd F A, Antonia S J, et al. First-line nivolumab (anti-PD-1; BMS-936558, ONO-4538) monotherapy in advanced NSCLC: safety, efficacy, and correlation of outcomes with PD-L1 status. Presented at: American Society of Clinical Oncology Annual Meeting; 2014 Jun. 3-7; Chicago, IL, USA; 2014. poster 38.
14. Rizvi N A, Hellmann M D, Snyder A, et al. Cancer immunology. Mutational landscape determines sensitivity to PD-1 blockade in non-small cell lung cancer. Science 2015; 348:124-8.
15. Eisenhauer E A, Therasse P, Bogaerts J, et al. New response evaluation criteria in solid tumours: revised RECIST guideline (version 1.1). Eur J Cancer 2009; 45:228-47.
16. Wakelee H A, Wang W, Schiller J H, et al.; for the Eastern Cooperative Oncology Group. Survival differences by sex for patients with advanced non-small cell lung cancer on Eastern Cooperative Oncology Group trial 1594. J Thorac Oncol 2006; 1:441-6.
17. Reck M, Rodriguez-Abreu D, Robinson A G, et al.; for the KEYNOTE-024 Investigators. Pembrolizumab versus chemotherapy for PD-L1-positive non-small-cell lung cancer. N Engl J Med 2016; 375:1823-33.
18. Remon J, Besse B, Soria J C. Successes and failures: what did we learn from recent first-line treatment immunotherapy trials in non-small cell lung cancer? BMC Med 2017; 15:55.
19. Retseck J, VanderWeele R, Lin H M, Lin Y, Butterfield L H, Tarhini A A. Phenotypic and functional testing of circulating regulatory T cells in advanced melanoma patients treated with neoadjuvant ipilimumab. J Immunother Cancer 2016; 4:38.
20. Tarhini A A, Edington H, Butterfield L H, et al. Immune monitoring of the circulation and the tumor microenvironment in patients with regionally advanced melanoma receiving neoadjuvant ipilimumab. PLOS One 2014; 9: e87705.
21. Curran M A, Montalvo W, Yagita H, Allison J P. PD-1 and CTLA-4 combination blockade expands infiltrating T cells and reduces regulatory T and myeloid cells within B16 melanoma tumors. Proc Natl Acad Sci USA 2010; 107:4275-80.
22. Hellmann M D, Rizvi N A, Goldman J W, et al. Nivolumab plus ipilimumab as first-line treatment for advanced non-small-cell lung cancer (CheckMate 012): results of an open-label, phase 1, multicohort study. Lancet Oncol 2017; 18:31-41.
23. Socinski M A, Creelan B, Horn L, et al. CheckMate 026: A phase 3 trial of nivolumab vs investigator's choice of platinum-based doublet chemotherapy as first-line therapy for stage IV/recurrent programmed death ligand 1-positive NSCLC. Presented at: European Society for Medical Oncology 2016 Congress; 2016 Oct. 7-11; Copenhagen, Denmark; 2016.

1. A method of treating a tumor in a subject in need thereof, comprising administering to the subject an antibody or antigen-binding portion thereof that binds specifically to a Programmed Death-1 (PD-1) receptor and inhibits PD-1 activity (“an anti-PD-1 antibody”) wherein the tumor is identified as having a tumor mutational burden (TMB) status that is a high TMB, wherein the high TMB has a score of at least 243 genetic alterations as measured by an exome sequencing assay.

2. The method of claim 1, wherein the TMB status of the subject is measured prior to the treatment.

3. The method of claim 1, wherein the TMB status is determined by sequencing nucleic acids in the tumor and identifying a genomic alteration in the sequenced nucleic acids.

4. The method of claim 3, wherein the tumor has one or more genomic alterations comprising:
(a) a somatic mutation;
(b) a nonsynonymous mutation;
(c) a missense mutation;
(d) a base pair substitution;
(e) a base pair insertion;
(f) a base pair deletion;
(g) a copy number alteration (CNA);
(h) a gene rearrangement; or
(i) any combination of (a)-(h).

5-10. (canceled)

11. The method of claim 1, wherein the tumor comprises lung cancer, renal cell carcinoma, ovarian cancer, colorectal cancer, gastrointestinal cancer, esophageal cancer, bladder cancer, or melanoma.

12. The method of claim 1, wherein the anti-PD-1 antibody comprises nivolumab.

13. The method of claim 1, wherein the anti-PD-1 antibody is administered at a dose of from about 200 mg to about 1200 mg once every 2, 3, or 4 weeks.

14. The method of claim 12, wherein the anti-PD-1 antibody is administered at a dose of about 200 mg, about 240 mg, or 480 mg.

15. The method of claim 1, further comprising administering to the subject an antibody or antigen-binding portion thereof that specifically binds to cytotoxic T-lymphocyte-associated protein 4 (CTLA-4) (“an anti-CTLA-4 antibody”).

16. A method of identifying a subject suitable for an immunotherapy comprising an anti-PD-1 antibody, the method comprising measuring a TMB status of a biological sample of a subject who is afflicted with a tumor, wherein the TMB status is measured by an exome sequencing assay; wherein the TMB status comprises a score of at least 243 genetic alterations; and wherein the tumor is further identified as having greater than or equal to 50% of the tumor cells expressing PD-L1.

17. The method of claim 1, wherein the anti-PD-1 antibody comprises pembrolizumab.

18. The method of claim 12, wherein the anti-PD-1 antibody is administered at a dose of about 240 mg administered once about every 2 weeks.

19. The method of claim 17, wherein the anti-PD-1 antibody is administered at a dose of about 200 mg administered once about every 3 weeks.

20. The method of claim 12, wherein the anti-PD-1 antibody is administered at a dose of about 360 mg administered once about every 3 weeks.

21. The method of claim 12, wherein the anti-PD-1 antibody is administered at a dose of about 480 mg administered once about every 4 weeks.

22. The method of claim 12, wherein the anti-PD-1 antibody is administered at a dose of about from about 0.1 mg/kg to about 10.0 mg/kg body weight once every 2, 3, or 4 weeks.

23. The method of claim 16, further comprising administering an anti-PD-1 antibody.

24. The method of claim 23, wherein the anti-PD-1 antibody comprises nivolumab or pembrolizumab.

25. The method of claim 23, wherein the anti-PD-1 antibody comprises nivolumab administered at a dose of about 240 mg once every 2 weeks.

26. The method of claim 23, wherein the anti-PD-1 antibody comprises nivolumab administered at a dose of about 480 mg once every 4 weeks.
